(19) United States
(12) Patent Application Publication
(10) Pub. No.: US 2024/0255506 A1
(43) Pub. Date: Aug. 1, 2024

(54) PAIRED-CELL PROFILING SYSTEMS FOR HIGH-THROUGHPUT SCREENING SYNCYTIUM FORMATION

(71) Applicants: Versitech Limited, Hong Kong (CN); Centre for Oncology and Immunology Limited, Hong Kong (CN); Advanced Biomedical Instrumentation Centre Limited, Hong Kong (CN)

(72) Inventors: Siu Lun Wong, Hong Kong (CN); Ching Gee Choi, Hong Kong (CN); Ho Cheung Shum, Hong Kong (CN); Wai Fong Chan, Hong Kong (CN); Bei Wang, Hong Kong (CN); Lang Nan, Hong Kong (CN)

(73) Assignees: Versitech Limited, Hong Kong (CN); Centre for Oncology and Immunology Limited, Hong Kong (CN); Advanced Biomedical Instrumentation Centre Limited, Hong Kong (CN)

(21) Appl. No.: 18/419,328

(22) Filed: Jan. 22, 2024

Related U.S. Application Data
(60) Provisional application No. 63/481,830, filed on Jan. 27, 2023.

(51) Int. Cl. G01N 33/569; C12N 9/22; C12N 15/11; G01N 21/64

(52) U.S. Cl. CPC G01N 33/56983; C12N 9/22; C12N 15/11; G01N 21/6486; C12N 2310/20; G01N 2333/165

(57) ABSTRACT

Provided is a high throughput screening method for identifying cell fusion or syncytium formation potential. In particular, the cell fusion or syncytium formation potential of a viral spike protein. Also provided is a method for identifying a cellular factor that promotes spike protein-induced syncytium formation. Also provided is a method of inhibiting or suppressing syncytium formation induced by a viral spike protein.

This application claims priority to U.S. provisional application No. 63/481,830 filed on Jan. 27, 2023, which is incorporated by reference herein in its entirety.

### SEQUENCE LISTING

The instant application contains a Sequence Listing which has been submitted electronically in XML file format and is hereby incorporated by reference in its entirety. Said XML copy, created on Jan. 19, 2024, is named 10030_011718-US1_SLxml and is 175,565 bytes in size.

### 1. FIELD

The disclosure is generally directed to a high throughput screening method of genetic and cellular drivers of syncytium formation.

### 2. BACKGROUND

Severe acute respiratory syndrome coronavirus 2 (SARS-COV-2), which caused the worldwide COVID-19 pandemic, has been continuously evolving since its emergence, and numerous variants with different degrees of infectivity and lethality have been arising. Predicting how the variants' pathogenicity changes with their acquired mutations and understanding their interactions with host cell factors are critically important for formulating strategies to confront their threats to global public health and prepare for future outbreaks.

SARS-COV-2 infects cells by binding its surface spike protein with the host cell receptor angiotensin-converting enzyme 2 (ACE2)¹. The spike protein is a viral fusogen that allows virus-cell fusion and cell-cell fusion following cleavage and priming by surface or endosomal proteases of the target cells^{2,3}. Extensive lung tissue damage, with the presence of large multinucleated syncytial pneumocytes formed due to cell-cell fusion, is characterized in post-mortem samples from individuals who died of COVID-19 and syncytia are considered as a frequent feature of severe COVID-19⁴. Syncytia are formed by two or more cells fusing. SARS-COV-2 induces syncytium formation when the spike protein on the surface of an infected cell interacts with receptors on neighboring cells. Syncytia potentially contribute to pathology by facilitating viral dissemination, cytopathicity, lymphocyte elimination and inflammatory response^{5,6}. Via syncytia, SARS-COV-2 can also enter cells through cell fusion between infected and uninfected cells, thus contributing to the spread of the virus through cell-to-cell transmission⁷.

The presence of multinucleated syncytia in the lung tissues of COVID-19 patients appears to be primarily determined by the fusogenicity of the spike protein of SARS-COV-2. It was confirmed that the SARS-COV-2 spike protein alone is sufficient to drive syncytium formation, as in vitro co-culture assay showed that the cells expressing spike only, in the absence of other SARS-COV-2 viral proteins, could fuse with the neighboring ACE2-expressing cells and form syncytia³. A cryo-electron microscopy-determined structural model of the trimeric spike has revealed that spike is expressed on the surface of the virions as a trimer, leading to the crown-like appearance⁸. Many studies, including protein structure and biochemical studies, have been performed to understand how the spike protein works^{9,10}. The spike protein, a heavily glycosylated type I transmembrane protein with 1,273 amino acids, comprises two functional subunits, S1 and S2. S1 contains a receptor-binding domain (RBD) responsible for viral attachment to the host receptor, and the S2 subunit contains a protease cleavage site (S2′), a hydrophobic fusion peptide and two heptad repeat regions responsible for membrane fusion for entry into host cells. At the S1/S2 boundary, there is a polybasic cleavage site that is not found in SARS-COV-1 or other SARS-like coronaviruses¹¹. S1/S2 is cleaved during virus assembly in virus-producing cells by furin. When infecting target cells, the spike protein interacts with ACE2. The pre-cleaved spike is further cleaved by transmembrane serine protease 2 (TMPRSS2) to allow plasma membrane entry. The spike that is not cleaved by TMPRSS2 is endocytosed and subsequently cleaved by endosomal proteases such as cathepsin L for endosomal entry. Following up on these analyses, the next important step is to systematically map the sequence-to-phenotype relationship of the SARS-CoV-2 spike protein and determine how SARS-COV-2 spike mutations affect its ability to induce fusion and disease severity.

During viral evolution, Alpha, Beta and Delta strains of SARS-COV-2 spike, considered variants of concern (VOCs), emerged, forming more and larger syncytia in the infected cells than their parental D614G strain (designated as wild-type (WT) in this disclosure)¹². In molecular-level studies, P681H and P681R mutations found in the S1/S2 cleavage site of the Alpha and Delta variants, respectively, were shown to increase syncytium formation^{12,13}. The two mutations may expose the furin cleavage site of spike such that its proteolytic cleavage occurs more readily, stimulating subsequent fusion^{13,14}. The Omicron variant of the spike protein also promotes the formation of obvious syncytia, although the extent is less than that induced by the Delta variant^{15,16}. The fusion abilities vary among the ever-growing numbers of Omicron subvariants, which have acquired additional mutations¹⁷. Rapid characterization of the fusion ability of emerging spike variants will aid the identification of mutations of concern.

In the past, methods for high-throughput screening of cell-cell fusion utilizing High-content imaging are less precise since it is using the area of syncytium/fraction of fused cells under the microscope. For genetic screens, >3,000 drugs were screened in ref. 19 and ˜6,000 drugs and >30 spike protein variants were screened in ref. 20. Screening of genetic variants and perturbations requires the individual library constructs to be library constructs to be generated and delivered into microwell arrays for imaging. With an imaging throughput of about 30 s per well, screening a genome-wide CRISPR library of 37,722 sgRNAs is estimated to take about 13 days. It also has potentially higher variation. There may be a lag time in cell fixation/image acquisition over a large number of samples to evaluate the fast cellular process. The non-pooled experimental setup may be more subject to technical variations such as cell density.

Large-scale screening to characterize cell-cell interaction/fusion instead of single-cell behavior is technically more challenging, as grouping of two different cell types for characterization of their interaction requires compartmentalization methods such as droplet microfluidics¹⁸. In all previous studies, individual spike variants were constructed one-by-one, and their syncytium-forming potential was individually monitored by microscopy after co-culturing two types of cells: spike-presenting sender cells and fusion-permissible receiver cells³ and quantified on the basis of the area of syncytium formed under the microscope, which limits precision. The scalability of those experiments constrains its utility for characterizing the many subvariants and emerging variants. High-content imaging could be automated for large-scale profiling experiments^{19,20}, but there is lag time in imaging the first well until the last one among the tens of thousands. The extended time delay is not ideal for capturing rapidly progressing cellular processes, such as syncytium formation that can be seen within an hour after the sender and receiver cells encounter²⁰, among a large library of variants for their head-to-head comparison (Table 1). Moreover, screening of unique genetic variants/perturbations requires each of the library constructs to be individually built and delivered into single wells of the arrays, which is technically demanding and expensive. Cell-cell fusion rate across experiments can also be greatly affected by cell density and sender-to-receiver cell ratio; thus, non-pooled assays are more subject to technical variations than pooled experiments when doing comparisons across many variants.

However, before this work, to the best of our knowledge, no large-scale quantitative analysis that directly reveals how mutations of the spike protein affect the cell-cell fusion process has been reported.

In addition, how the spike protein hijacks the host cell machinery to fuse cells together is not well understood. Understanding how spike-induced cell-cell fusion occurs may allow us to develop effective strategies to mitigate syncytium formation induced by SARS-COV-2. Several genome-wide CRISPR screens were carried out in the search for host factors required for SARS-COV-2 infection22-28, in which live SARS-COV-2 was used to infect the cells; a suite of its viral proteins were expressed and each of them could hijack various machineries of the host cells to aid viral replication, assembly, release and cell death. Currently, no systematic studies pinpoint the cellular determinants of syncytium formation to fill in this knowledge gap.

### 3. SUMMARY

Mapping mutations and discovering cellular determinants that cause the spike protein of severe acute respiratory syndrome coronavirus 2 (SARS-COV-2) to induce infected cells to form syncytia would facilitate the development of strategies for blocking the formation of such cell-cell fusion. Here we describe high-throughput screening methods based on droplet microfluidics and the size-exclusion selection of syncytia, coupled with large-scale mutagenesis and genome-wide knockout screening via clustered regularly interspaced short palindromic repeats (CRISPR), for the large-scale identification of determinants of cell-cell fusion. We used the methods to perform deep mutational scans in spike-presenting cells to pinpoint mutable syncytium-enhancing substitutions in two regions of the spike protein (the fusion peptide proximal region and the furin-cleavage site). We also used a genome-wide CRISPR screen in cells expressing the receptor angiotensin-converting enzyme 2 to identify inhibitors of clathrin-mediated endocytosis that impede syncytium formation, which we validated in hamsters infected with SARS-COV-2. Finding genetic and cellular determinants of the formation of syncytia may reveal insights into the physiological and pathological consequences of cell-cell fusion.

We established droplet microfluidics and size-exclusion selection strategies to study cell-cell fusion, and couple them with large-scale mutagenesis and CRISPR screening. In this study, we experimentally scanned mutations over two regions of SARS-COV-2 spike: the fusion peptide proximal region and the furin cleavage-site region, for their impact on spike's syncytium-forming potential, as well as screened through the entire human genome for host factors that are crucial for spike-induced syncytium formation. We developed these methods in human cells, which offer the advantage of conferring the post-translational modifications, including glycosylation, to spike that it acquires under physiological conditions to induce syncytia. Our work provides opportunities to rapidly understand the pathological consequence (that is, syncytium formation) of SARS-COV-2 genetic variation by profiling a large panel of variants, enabled by our methods, and identifies candidate genetic factors in human cells that could serve as potential therapeutic targets for combating syncytium formation. Without these methods, it would be technically challenging to study these targets en masse.

Provided herein is a high throughput screening method for identifying cell fusion or syncytium formation potential of a viral spike protein, the method comprising the steps of:
(a) preparing a plurality of spike protein variants;
(b) incorporating said spike protein variants into a plurality of spike-presenting sender cells, each cell comprising a spike protein variant from said plurality of spike protein variants;
(c) contacting said spike-presenting sender cells with a plurality of receiver cells expressing angiotensin-converting enzyme 2 (ACE2) to obtain a mixture;
(d) incubating the mixture obtained in step (c) in a microfluidic system comprising multiple droplets, each droplet comprising an individual sender cell and an individual receiver cell; and
(e) determining the syncytium-forming potential of the spike protein variants in the droplets.

In certain embodiments, the viral spike protein is a respiratory virus spike protein.

In certain embodiments, the respiratory virus is a coronavirus.

In certain embodiments, the respiratory virus is selected from the group consisting of severe acute respiratory syndrome (SARS) coronavirus (SARS-COV), SARS-COV-2 (COVID-19), Middle East Respiratory Syndrome (MERS), respiratory syncytial virus (RSV), influenza virus, parainfluenza virus (PIV), pneumovirus (PMV), metapneumovirus (MPV), respirovirus, and rubulavirus.

In certain embodiments, the respiratory virus is SARS-COV-2 (COVID-19).

In certain embodiments, the plurality of spike protein variants is prepared by site saturation mutagenesis (SSM).

In certain embodiments, the respiratory virus is SARS-COV-2, and wherein the SSM is over the fusion peptide proximal region (FPPR) of SARS-COV-2.

In certain embodiments, the respiratory virus is SARS-COV-2, and wherein the SSM is over the furin cleavage-site region of SARS-COV-2.

In certain embodiments, the syncytium-forming potential of the spike protein variants is determined by measuring a signal generated upon fusion of the sender and receiver cells.

In certain embodiments, the signal is a fluorescent, bioluminescent, chemiluminescent or a radioactive signal.

In certain embodiments, the signal is a green fluorescent protein (GFP) signal that is generated upon fusion of the sender and receiver cells.

In certain embodiments, the syncytium-forming potential of the spike protein variants is determined using deep mutational scanning (DMS) in the spike-presenting sender cells.

In certain embodiments, the method further comprises the step of enriching the spike variants having enhanced syncytium-formation potential.

In certain embodiments, the method further comprises the step of analyzing the abundance of each variant by sequencing spike variants having enhanced syncytium-formation potential.

Provided is a high throughput screening method for identifying cell fusion or syncytium formation potential of a viral spike protein, the method comprises the step of
(a) preparing a plurality of spike protein variants;
(b) incorporating said spike protein variants into a plurality of spike-presenting sender cells, each cell comprising a spike protein variant from said plurality of spike protein variants;
(c) contacting said spike-presenting sender cells with a plurality of receiver cells expressing angiotensin-converting enzyme 2 (ACE2) to obtain a mixture;
(d) performing size-exclusion selection by passing the mixture through a cell strainer; and
(e) determining the syncytium-forming potential of the spike protein variants.

In certain embodiments, the method further comprises the step of collecting a first syncytia population that is retained on said strainer, and a second syncytia population that passes through the strainer.

In certain embodiments, the method further comprises the step of enriching the spike variants having enhanced syncytium-formation potential from the first and second syncytia populations.

In certain embodiments, the method further comprises the step of analyzing the abundance of each variant by sequencing spike variants having enhanced syncytium-formation potential in the first and second syncytia populations.

In certain embodiments, the plurality of spike protein variants is prepared by site saturation mutagenesis (SSM).

In certain embodiments, the SSM is over the fusion peptide proximal region (FPPR) or the furin cleavage site of SARS-COV-2.

In certain embodiments, the syncytium-forming potential of the spike protein variants is determined by measuring a signal generated upon fusion of the sender and receiver cells.

In certain embodiments, the signal is a fluorescent, bioluminescent, chemiluminescent or a radioactive signal.

In certain embodiments, the signal is a green fluorescent protein (GFP) signal that is generated upon fusion of the sender and receiver cells.

In certain embodiments, the syncytium-forming potential of the spike protein variants is determined using deep mutational scanning (DMS) in the spike-presenting sender cells.

Provided is a method for identifying a cellular factor that promotes spike protein-induced syncytium formation, the method comprising the steps of:
(a) preparing a plurality of spike protein variants;
(b) incorporating said spike protein variants into a plurality of spike-presenting sender cells, each cell comprising a spike protein variant from said plurality of spike protein variants;
(c) contacting said spike-presenting sender cells with a plurality of receiver cells expressing angiotensin-converting enzyme 2 (ACE2) and single guide RNA (sgRNA)-Cas9 to obtain a mixture;
(d) isolating unfused receiver cells from the mixture;
(e) comparing the sgRNA abundance in unmixed receiver cells and isolated unfused receiver cells from step (d); and
(f) identifying a factor in the receiver cell that promotes syncytium formation.

In certain embodiments, the plurality of spike protein variants is prepared by site saturation mutagenesis (SSM).

In certain embodiments, the SSM is over the fusion peptide proximal region (FPPR) or the furin cleavage site of SARS-COV-2.

In certain embodiments, the syncytium-forming potential of the spike protein variants is determined by measuring a signal generated upon fusion of the sender and receiver cells.

In certain embodiments, the signal is a fluorescent, bioluminescent, chemiluminescent or a radioactive signal.

In certain embodiments, the signal is a green fluorescent protein (GFP) signal that is generated upon fusion of the sender and receiver cells.

In certain embodiments, the host factor comprises a core regulator of clathrin-mediated endocytosis (CME).

In certain embodiments, the CME regulator is selected from the group consisting of FCHO2, AP2M1, CAB39, RNF2 and GBP6.

Provided herein is a high throughput screening method for identifying cell fusion or syncytium formation potential of a first protein and a second protein in a biological system, the method comprising the steps of:
(a) preparing a plurality of first protein variants;
(b) incorporating said first protein variants into a plurality of first protein-presenting cells, each cell comprising a first protein variant from said plurality of first protein variants;
(c) contacting said first protein presenting cells with a plurality of cells expressing said second protein to obtain a mixture;
(d) incubating the mixture obtained in step (c) in a microfluidic system comprising multiple droplets, each droplet comprising an individual first protein presenting cell and an individual cell expressing said second protein; and
(e) determining the syncytium-forming potential of the first protein variants in the droplets.

In certain embodiments, the biological system is a virus, tumor, maternal-fetal material exchange in the placenta, muscle contraction and bone resorption.

In certain embodiments, the virus is selected from the group consisting of human immunodeficiency virus, Herpesviridae, respiratory syncytial virus and Coronaviridae.

In certain embodiments, the first protein is from a tumor and the second protein is from normal somatic cells or dendritic cells.

In certain embodiments, the first protein or second protein is from multinucleated cells selected from the group consisting of syncytiotrophoblasts, myotybes and osteoclasts.

In certain embodiments, the first protein is from B cells and the second protein is from myeloma cells that produces hydridoma.

In certain embodiments, the first protein is from human embryonic stem cells and the second protein is from somatic cells.

In certain embodiments, the syncytium-forming potential of the first protein variants is determined by measuring a signal generated upon fusion of the first protein presenting cells and cells expressing said second protein.

In certain embodiments, the signal is a fluorescent, bioluminescent, chemiluminescent or a radioactive signal.

In certain embodiments, the signal is a green fluorescent protein (GFP) signal that is generated upon fusion of the first protein presenting cells and cells expressing said second protein.

In certain embodiments, the syncytium-forming potential of the first protein variants is determined using deep mutational scanning (DMS) in the first protein-presenting cells.

In certain embodiments, the method further comprises the step of enriching the first protein variants having enhanced syncytium-formation potential.

In certain embodiments, the method further comprises the step of analyzing the abundance of each variant by sequencing first protein variants having enhanced syncytium-formation potential.

In certain embodiments, the mixture is further comprises single guide RNA (sgRNA)-Cas9 of step (c), and further comprises the steps of: (f) isolating unfused cells expressing said second protein from the mixture; (g) comparing the sgRNA abundance in unmixed cells expressing said second protein and the isolated unfused cells expressing said second protein from step (d).

Provided is a high throughput screening method for identifying cell fusion or syncytium formation potential of a first protein and a second protein in a biological system, the method comprising the steps of:
(a) preparing a plurality of first protein variants;
(b) incorporating said first protein variants into a plurality of first protein-presenting cells, each cell comprising a first protein variant from said plurality of first protein variants;
(c) contacting said first-presenting cells with a plurality of cells expressing a second protein to obtain a mixture;
(d) performing size-exclusion selection by passing the mixture through a cell strainer; and
(e) determining the syncytium-forming potential of the first protein variants.

In certain embodiments, the method further comprises the step of collecting a first syncytia population that is retained on said strainer, and a second syncytia population that passes through the strainer.

In certain embodiments, the method further comprises the step of enriching the first protein variants having enhanced syncytium-formation potential from the first and second syncytia populations.

In certain embodiments, the method further comprises the step of analyzing the abundance of each variant by sequencing the first protein variants having enhanced syncytium-formation potential in the first and second syncytia populations.

In certain embodiments, the plurality of the first protein variants is prepared by site saturation mutagenesis (SSM).

In certain embodiments, the syncytium-forming potential of the first protein variants is determined by measuring a signal generated upon fusion of the first protein presenting cells and cells expressing said second protein.

In certain embodiments, the signal is a fluorescent, bioluminescent, chemiluminescent or a radioactive signal.

In certain embodiments, the signal is a green fluorescent protein (GFP) signal that is generated upon fusion of the first protein presenting cells and cells expressing said second protein.

In certain embodiments, the mixture in step (c) further comprises single guide RNA (sgRNA)-Cas9, and further comprising the steps of (f) isolating unfused cells expressing said second protein from the mixture; (g) comparing the sgRNA abundance in unmixed cells expressing said second protein and the isolated unfused cells expressing said second protein.

In certain embodiments, the syncytium-forming potential of the first protein variants is determined using deep mutational scanning (DMS) in the first protein-presenting cells.

Provided herein is a method of inhibiting or suppressing syncytium formation induced by a viral spike protein, the method comprises the step of administering to a viral cell comprising said spike protein an inhibitor of CME inhibitor.

### 4. BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1C. A droplet microfluidics-based system for high-throughput screening of syncytium formation. A. Co-culture of spike-expressing sender cells (mCherry⁺) and ACE2-expressing receiver cells (BFP⁺) resulted in syncytium formation (GFP⁺) due to cell-cell fusion. Scale bar, 50 μm. B. Workflow for cell encapsulation and incubation in droplets and the detection/collection of GFP⁺ syncytium (boxed). Side scatter (SSC) was used to identify the cell population. C. Representative images of sender cell-receiver cell fusion in droplets. A fusion rate of 57.9%±3.3% (data shown are mean±s.d., n=6 biological replicates, with P value from unpaired two-tailed Student's t-test) was measured by FACS after 24 h of incubation in droplets, which is comparable to the bulk setting (55.2%±4.3%) in regular co-culture experiments without using droplet microfluidics. Red fluorescent protein (RFP) refers to the mCherry signal detected under FACS.

FIGS. 2A-2F. Deep mutational scanning of SARS-COV-2 spike's syncytium-forming potential using the droplet microfluidics-based system. A. Workflow for deep mutational scanning in sender cells using a droplet microfluidics-based system. GFP⁺ syncytia containing the fusion-competent spike variants are sorted out for NovaSeq-based sequencing. B. Heat maps depicting how all single mutations affect the syncytium-forming potential of spike's FPPR and furin cleavage site regions. Squares are coloured by mutational effect (that is, FC) according to colour bars on the right, with red and purple indicating syncytium-enhancing and inhibiting substitutions, respectively. FC represents each variant's relative abundance in the sorted GFP-positive cell pool vs the cell pool before mixing and is normalized to WT. Spike variants with increased syncytium-formation potential were enriched (with fold change >1), while those with decreased syncytium-formation potential were depleted (with fold change <1). Grey cross, mutations with no measurement; black dot, SARS-COV-2 WT amino acid; *, stop codon. C. Validation of syncytium-enhancing and inhibiting mutations at the FPPR in WT D614G and Omicron spike. GFP-split complementation system assay was applied and representative images are shown. D. Quantification of the syncytium area (top) and average size of syncytium (bottom) for spike variants in c. Data shown are mean±s.d.; n=6 biological replicates. P values coloured in blue indicate statistical comparison between individual spike D614G and Omicron mutants to the D614G WT. P values coloured in red indicate statistical comparison between individual Omicron mutants to the omicron WT. Statistical significance was determined using one-way ANOVA. ***P<0.001, ****P<0.0001. E. Mutability score for individual position of spike protein studied in b and RBD. The number indicates the position of the amino acid on the spike protein. The letter indicates the wild-type amino acid on the position. F. Mutational effects on syncytium formation revealed by the pooled screen using the droplet microfluidics-based system and individual validation assay for the spike variants. Black dots, validated spike FPPR variants.

FIGS. 3A-3D. A size-exclusion selection-based strategy for high-throughput screening of syncytium formation. A. Workflow for DMS in sender cells using a size-exclusion selection-based system. Large syncytia are collected using the cell strainer, while small GFP+ syncytia are collected by FACS. The two populations containing the fusion-competent spike variants are subjected to NovaSeq-based sequencing. B. Heat maps depicting how all single mutations affect the syncytium-forming potential of spike's FPPR and furin cleavage site regions. Squares are coloured by mutational effect (that is, FC) according to colour bars on the right, with red and purple indicating syncytium-enhancing and inhibiting substitutions, respectively. FC represents each variant's relative abundance in the syncytia collected using the cell strainer or FACS vs the cell pool before mixing and is normalized to WT. Spike variants with increased syncytium-formation potential were enriched (with fold change >1), while those with decreased syncytium-formation potential were depleted (with fold change <1). Grey cross, mutations with no measurement; black dot, SARS-COV-2 amino acid; *, stop codon. C. Correlation of the profiling results (that is, FC) obtained using droplet microfluidics-based and size-exclusion selection-based strategies. Validated syncytium-enhancing and inhibiting mutations/residues are highlighted and labelled. D. Mutational effects on syncytium formation revealed by the pooled screen using the size-exclusion selection-based system and individual validation assay for the spike variants. FIGS. 4A-4H. Genome-wide CRISPR screen reveals host factors important for SARS-COV-2 spike-induced syncytium formation. A. Workflow for genome-wide CRISPR screening in receiver cells using a reverse selection approach. Large syncytia and small GFP+ syncytia were removed using cell strainer and FACS, respectively. Unfused A549-ACE2-SpCas9-GFP1-10-sgRNA cells were subjected to NovaSeq-based sequencing. B. Enrichment of sgRNAs in the unfused receiver cell population revealed by the RRA score and FC. FC represents each variant's relative abundance in the unfused receiver cell pool vs the unmixed cell pool and is normalized to WT. The sgRNA screen hits are highlighted in red and the known tumour suppressor genes are highlighted in blue. The genome-wide CRISPR screen data were collected from two biological replicates. C. Validation of screen hits using sgRNA-directed gene knockouts in A549-ACE2-SpCas9-GFP1-10 receiver cells. The HEK293T-GFP-11 sender cells used express WT D614G spike. D. Quantification of the syncytium area (left) and average size of syncytium (right) observed in c. Data shown are mean #s.d. (n=6 biological replicates). E. Quantitative PCR with reverse transcription (RT-qPCR) (E) and western blotting. (F). Results on CHC knockdown. G. and H. Inhibition of syncytium formation by CHC knockdown (G) and Pitstop2 treatment (H). GFP-split complementation system assay was applied as in C. Representative images are shown. Data shown are mean±s.d. (n=3 (E), n=6 (G) and n=9 (H) biological replicates). P values indicated were compared with safe harbour-targeting sgRNA (D) control (ctr) shRNA (e,g) or DMSO-treated control (H). Statistical significance was determined using one-way ANOVA. ****P<0.0001. Scale bars, 200 μm.

FIGS. 5A-5D. Clathrin-mediated endocytosis inhibitors impede SARS-COV-2 spike-induced syncytium formation. A-D. GFP-split complementation system assay was applied using A549-ACE2-GFP1-10 (A, B) and Vero E6-GFP1-10 (C,D) as receiver cells. The HEK293T-GFP-11 sender cells used express WT D614G spike. Representative images are shown. Data shown are mean±s.d. (n=6 biological replicates). P values indicated were compared with DMSO-treated control. Statistical significance was determined using one-way ANOVA. ****P<0.0001. Scale bars, 200 μm.

FIGS. 6A-6G. Clathrin-mediated endocytosis inhibitors impede SARS-COV-2 live virus-induced syncytium formation. A. GFP-split complementation system assay was applied using Vero E6-GFP1-10 and Vero E6-GFP11 cells, and Vero E6-TMPRSS2-GFP1-10 and Vero E6-TMPRSS2-GFP11 cells (a), mixed at a 1:1 ratio. The cells were treated with the indicated drugs and infected with SARS-COV-2 D614G live virus. Representative images are shown. B. Quantification of the area of syncytium formation in a. Data shown are mean #s.d. (n=6 biological replicates). P values indicated were compared with DMSO-treated control. Statistical significance was determined using one-way ANOVA. C. RT-qPCR measurements of SARS-COV-2 RdRp levels in CME inhibitor-treated Vero E6 and Vero E6-TMPRSS2 cells. The RdRp levels in both cell lysate and supernatant after SARS-COV-2 D614G virus infection were measured. D-G, Lung tissues of CME inhibitor/DMSO-treated, SARS-COV-2 D614G virus-infected hamsters were collected (D). The RdRp level was measured by RT-qPCR (E). The infectious virus titres were measured by plaque assay (F). The presence of syncytium-like multinucleated cells was detected by immunofluorescence (IF) staining and indicated by arrows in the representative images (G). Lung tissues were stained by antibodies against SARS-COV-2 nucleocapsid protein (SARS-COV-2 N) (green) and sodium potassium ATPase (red). Nuclei were stained with DAPI (blue). Multinucleated cells were indicated by white arrows in the representative images (i, ii, iii). Scale bars, 200 μm (A) and 100 μm (G). In C, E and F, data shown are mean±s.d.; n=4 (C), n=6 (E, F) biological replicates. P values indicated were compared with DMSO-treated control. Statistical significance was determined using one-way ANOVA. ****P<0.0001. Schematic diagrams in A and D were created with BioRender.com.

FIGS. 7A-7D. Establishment of systems and conditions for sender-cell and receiver-cell encapsulation in droplets. A) Visualization of cell-cell fusion using GFP-split complementation system assay. GFP11 in Spike-expressing sender cells and GFP1-10 in ACE2-expressing receiver cells are nonfluorescent by themselves, while coculture and fusion of the sender and receiver cells resulted in reconstituted GFP that becomes fluorescent upon complementation. Created with BioRender.com B) Design of microfluidics device for generation of monodispersed droplets with human cells. Conditions used: continuous phase (Qc): HFE with 0.5% (w/w) of surfactant; dispersed phase (Qd): HEK293T cells (2.5×106 cells/ml) in culture medium (18% OptiPrep); flow rate: Qc:Qd=9,000:6,000 μL/hour. C) Droplet occupancy increased with the sender/receiver cell concentration used and aligned with Poisson distribution. D) Droplet co-occupancy in different mixing concentrations of the sender and receiver cells.

FIGS. 8A-8D. Deep mutational scanning of the ACE2-binding ability of the SARS-COV-2 Spike. A) Representative images showing the syncytium-forming potential of Omicron Spike variants. The fusion-defective HexaPro mutant and the Delta variant were included for comparison. (B) Heatmap depicting how all single mutations affect the ACE2 binding of Spike's FPPR. Squares are colored by mutational effect according to scale bars on the right, with red and purple indicating ACE2 binding-enhancing and inhibiting substitutions, respectively. The mutations with no measurement are in gray cross. The SARS-COV-2 amino acid is indicated with a black dot. Stop codon is indicated with *. The FC value represents the ACE2 binding ability for each of the single mutations in the Spike FPPR library. It is calculated as the fold change comparing each variant's relative abundance in FACS-sorted (that is, AF405-positive) ACE2-bound cell pool versus the unsorted cell pool and is normalized to wild-type. High reproducibility of the profiling result was detected between two biological replicates. C) Correlation of the syncytium-forming potential and the ability of ACE2 binding of all single mutations of Spike's FPPR. Mutants validated are highlighted and labeled. R is the Pearson correlation coefficient. (D) Cell surface staining of Spike FPPR variants in HEK293T sender cells. Anti-Spike antibodies against its S2 and S1 subunit were used to evaluate the total surface expression of Spike protein and the level of S1-cleaved Spike on cell surface, respectively. Ctr represents the control cells without Spike expression.

FIGS. 9A-9C. Individual validation of FPPR variants of Spike. (A) GFP-split complementation system was applied. A549-ACE2-GFP1-10 receiver cells and HEK293T-GFP-11 sender cells that express WT D614G Spike were used. Representative images are shown. B) Quantification of the syncytium area and average size of syncytium for Spike variants in (A). Data shown are mean±SD (n=6). P-values indicated were compared with the D614G WT. Statistical significance was determined using one-way ANOVA. ns: no significance. P<0.05, **P<0.01, ***P<0.001, ****P<0.0001. n indicates the number of biological replicates. C) Correlation of the syncytium area and average size of syncytium quantified for the Spike variants.

FIGS. 10A-10B. Structure models of K854H and A846W mutants of the SARS-COV-2 Spike. A-B) Parental D614 Spike (PDB: 6XR8), D614G Spike (PDB: 7KRQ), Omicron Spike (PDB: 7TO4) structures were used for the molecular modelling of K854H (A) and A846W (B) mutants.

FIGS. 11A-11D. Validation of syncytium-enhancing mutations at the furin cleavage site region in D614G Spike. A) GFP-split complementation system was applied for assessing the syncytium-enhancing mutations at the furin cleavage site region in D614G Spike. A549-ACE2-GFP1-10 receiver cells and HEK293T-GFP-11 sender cells that express WT D614G Spike were used. Representative images are shown. B) Quantification of the syncytium area and average size of syncytium for Spike variants in (A). Data shown are mean±SD (n=9). P-values indicated were compared with the D614G WT. C-D) Cell surface staining of Spike furin cleavage site variants in HEK293T sender cells. Anti-Spike antibodies against its S2 and S1 subunit were used to evaluate the total surface expression of Spike protein and the level of S1-cleaved Spike on cell surface, respectively. Ctr represents the control cells without Spike expression. In (D), the TMPRSS2 and Furin groups are HEK293T sender cells overexpressed with TMPRSS2 and Furin, respectively. HEK293T sender cells used in (C) and the ‘none’ group in (D) express only minimal levels of TMPRSS2 and Furin. The percentage of Spike cleavage is determined by the proportion of S1-stained cells. Data shown are mean±SD (n=3). P-values indicated were compared with the D614G WT in each group (that is, Ctr/TMPRSS2/Furin). Statistical significance was determined using one-way ANOVA. ns: no significance. *P<0.05, **P<0.01, ***P<0.001, ****P<0.0001. n indicates the number of biological replicates.

FIGS. 12A-12E. Quality assessment of the genome-wide CRISPR screen. A) Distribution of sgRNA reads in the plasmid pool extracted from E. coli and unmixed A549-ACE2-Cas9-GFP1-10-sgRNA cell pool at 14-day post-infection. 99.8% and 98.4% of all expected sgRNAs were obtained in the plasmid and cell pools, respectively. B) High reproducibility of sgRNA representations was detected between two biological replicates before (that is, unmixed) and after (that is, unfused) selection. R is the Pearson correlation coefficient. (C) Correlation of −log 10(negative RRA score) generated by MAGeCK and JACKS score by comparing sgRNA abundance in library-infected A549-ACE2-Cas9-GFP1-10-sgRNA cells (14-day post-infection) and the plasmid pool. Known essential genes are labeled in red. D-E) GO enrichment analysis (D) and KEGG classification enrichment analysis (E) of the depleted genes in A549-ACE2-Cas9-GFP1-10-sgRNA cells at 14-day post-infection.

FIGS. 13A-13B. Knockout of FCHO2 and AP2M1 inhibits Omicron Spike-induced syncytium formation. A-B) Validation of screen hits using sgRNA-directed gene knockouts in A549-ACE2-Cas9 and Vero E6-TMPRSS2-Cas9 receiver cells. The sender cells used to express either WT D614G Spike or Omicron Spike. Data shown are mean±SD (n=6 for (A) and n=3 for (B)). P-values indicated were compared with safe harbor-targeting (A) or non-targeting (B) sgRNA control. Statistical significance was determined using one-way ANOVA. n indicates the number of biological replicates. ****P<0.0001.

FIGS. 14A-14C. A genome-wide CRISPR screen reveals host-factor knockouts that enhance SARS-COV-2 Spike-induced syncytium formation. (A) Depletion of sgRNAs in the unfused receiver cell population revealed by the RRA and JACKS scores. The sgRNA screen hits are highlighted in red, and the known essential genes are highlighted in blue. The genome-wide CRISPR screen data were collected from two biological replicates. B) Validation of screen hits using sgRNA-directed gene knockouts in A549-ACE2-Cas9-GFP1-10 receiver cells. The HEK293T-GFP-11 sender cells used express WT D614G Spike. Scale bar, 200 μm. C) Quantification of the syncytium area and average size of syncytium observed in (B). Data shown are mean±SD (n=6). P-values indicated were compared with safe harbor-targeting control. Statistical significance was determined using one-way ANOVA. n indicates the number of biological replicates. ***P<0.001, ****P<0.0001.

FIGS. 15A-15D. Library design and quality assessment for the Spike deep mutational scans. A. Protein sequence alignment of SARS-COV-2's FPPR and furin cleavage site regions with other coronaviruses. SARS-COV-2 reference strain (GenBank accession number: YP_009724390.1), SARS-COV (YP_009825051.1), MERS-COV (YP_009047204.1), Rousettus bat CoV (YP_006908642.1), Bat CoV CDPHE15 (YP_008439202.1), Bat CoV 1A (YP_001718605.1), RaTG13 (MN996532). FIG. 15A discloses SEQ ID NOS 143-156, respectively, in order of appearance. B. SARS-COV-2 mutations at FPPR and furin cleavage site regions reported in the COG-UK-ME. C. Cumulative distribution of sequencing reads for the DMS library of Spike variants in the plasmid pools extracted from E. coli and the infected sender cell pools collected immediately before mixing with the receiver cells. High coverage of the FPPR library within the plasmid (100%) and infected cell (99.2%) pools, as well the Furin cleavage site region library within the plasmid (100%) and infected cell pool (99.4%), were detected. D. High reproducibility of profiling results was detected between two biological replicates in assessing the syncytium-forming potential of Spike variants at the FPPR and furin cleavage site region. The FC value represents the fold change comparing each variant's relative abundance in GFP-positive cell pool versus the cell pool before mixing and is normalized to wild-type. R is the Pearson correlation coefficient.

FIG. 16. Mutability scores of all single mutations of the FPPR and furin cleavage site region of SARS-COV-2 Spike. The DCA and IND scores are plotted against the Fold Change (FC) obtained from the Spike DMS library profiling for each mutant. Mutants validated in this study are highlighted and labelled.

FIG. 17. Assay ranges in defining fusion-(in)competent variants using droplet microfluidics-based and size-exclusion selection-based strategies. Histograms showing the distribution of syncytium-forming potentials (measured by log 2 Fold Change (FC)) of the WT Spike variants with synonymous codons and those with stop codons, assayed via the droplet microfluidics-based system and the size exclusion-based systems.

FIG. 18. Testing a reverse selection approach to collect fusion-resistant cells. HEK293T cells expressing ACE2-GFP11 and HEK293T cells expressing Spike-GFP1-10 were mixed with either HEK293T expressing BFP-ACE2-GFP11 or HEK293T expressing BFP only in 1:1:1 ratio. The cocultured cells were trypsinized and passed through a 40 μm cell strainer to remove the fused cells. Cells were imaged before and after passing through the strainer, and representative images are shown. The percentage of BFP+ cells was measured by FACS.

FIGS. 19A-19B. RNA-seq and gene ontology enrichment analysis on AP2M1 and FCHO2 knockout A549-ACE2-Cas9 cells. A-B. Genes that are differentially expressed in AP2M1 (A) and FCHO2 (B) knockout (KO) cells when compared with control (ctr) cells were identified by RNA-seq analysis. Data were collected from three biological replicates. The up- and down-regulated genes were subjected to gene ontology (GO) enrichment analysis. The top 20 hits of biological processes in the GO enrichment analysis are shown.

FIG. 20. Cell viability after treatment with endocytosis inhibitors. Viability of the drug-treated A549-ACE2 and Vero E6 cells was determined by XTT assay and is normalized to the DMSO-treated control. P-values indicated were compared with DMSO treated control. Statistical significance was determined using one-way ANOVA.

FIGS. 21A-21E. CME inhibition does not affect Spike and ACE2 surface expressions and ACE2 binding. A. ACE2 surface expression in gene knockout A549-ACE2 cells. ACE2 surface staining was performed on the sgRNA-infected A549-ACE2-Cas9-BFP cells at 10 days post-infection, and the percentage of ACE2 positivity is normalized to that in the control cells infected with safe harbor-targeting sgRNA. B-C. Spike surface expression (B) and ACE2 binding (C) of AP2M1 and FCHO2 knockout HEK293T sender cells. D-e, Spike surface expression and ACE2 binding of drug-treated HEK293T sender cells, and the percentages of Spike and ACE2 positivity are normalized to that in the DMSO-treated control. Data shown are mean±SD (n=3), P-values indicated were compared with the safe harbor sgRNA controls (A-C) or DMSO treated control (D-E). ns represents no significant difference. Statistical significance was determined using one-way ANOVA.

FIGS. 22A-22D. Clathrin-mediated endocytosis inhibition reduces SARS-COV-2 virus RdRp level and Spike-pseudotyped virus susceptibility. A-c, qRT-PCR measurements of SARS-CoV-2 RdRp levels in CME inhibitor-treated (A). AP2M1 and FCHO2 knockout (B). and CHC knockdown (C) A549-ACE2 cells. A549-ACE2-Cas9 cells were used in (B). The RdRp levels in both cell lysate and supernatant after SARS-COV-2 D614G virus infection were measured. D. Spike-pseudotyped virus susceptibility test of AP2M1 and FCHO2 knockout A549-ACE2 and Vero E6-TMPRSS2 cells. The pseudovirus infectivity was determined by measuring the GFP⁺ cells within the sgRNA-containing (BFP+) A549-ACE2-Cas9 and Vero E6-TMPRSS2-Cas9 cell populations. Data shown are mean±SD (n=4 for (a-c); n=3 for (D)). P-values indicated were compared with DMSO-treated control (A), safe harbour-targeting sgRNA (B, D), or control (ctr) shRNA (C). Statistical significance was determined using one-way ANOVA.

FIG. 23. Clathrin-mediated endocytosis inhibitors impede SARS-COV-2 live virus-induced pathological damages in hamster lung tissues. Haematoxylin and cosin staining was used to examine the pathological damages of lung tissues after SARS-COV-2 infection. The bronchiole epithelium region (i) and alveolar space region (ii) were highlighted in the images. In the DMSO-treated hamsters, extensive bronchiolar epithelium damages including bronchiolar epithelial desquamation and intense peribronchiolar mononuclear cell infiltration were detected. Alveolar congestion, haemorrhage and infiltration were observed in the alveolar region. CPZ and Fluvoxamine treatment alleviated the virus-induced pathological damages in regions of bronchiolar epithelium and alveolar space, including milder infiltration and alveolar haemorrhage.

### 5. DETAILED DESCRIPTION

For any viral pathogen, infectious cycle begins with an entry into the host cell. For this step, many viruses exploit cellular endocytosis machinery or fuse at the cell membrane to deliver a viral genome inside the cells. The present disclosure provides a high throughput screening method for identifying cell fusion or syncytium formation potential of a virus entering into a host cell as well as early events associated with the virus entry for a range of viruses that are syncytium-forming or syncytia-forming viruses. These viruses mediate fusion of an infected host cell with neighboring cells leading to the formation of multi-nucleate enlarged cells called syncytia. In some embodiments, the present methods are useful for syncytium-forming viruses which include, but are not limited to, viruses of family-Coronaviridae (e.g., SARS-COV-2, MERS, SARS-COV etc.), Herpesviridae (HSV, HCMV etc.), Paramyxoviridae (Nipah, Hendra, Measles, RSV etc.), Retroviridae (HIV, HTLV etc.), Hepatitis C Virus, Ebola, Sendai, Reovirus (e.g., Orthoreoviruses and Aquareoviruses). In certain embodiment, the viruses are SARS-COV-2, parainfluenza, influenza, Herpes Simplex Virus, Zika, and Japanese Encephalitis Virus. The present disclosure encompasses all variants, strains, serotypes, wild-type, and mutant versions of the viruses disclosed herein.

### 5.1 Spike Protein and Spike Protein Variants

In the present application, the term “S protein”, also known as “Spike protein”, generally refers to the glycoprotein on the capsid surface of a coronavirus. SARS-COV-2 invades cells by binding its S protein to ACE2. The S protein is composed of 1273 amino acids and contains a transmembrane domain, including the peptide segment with the amino acids from the N-terminal or the 14th position to at least 1273 position of the coronavirus capsid surface glycoprotein, or the corresponding region from other SARS viruses. The S1 protein is the subunit 1 of S protein, which mainly refers to the segment with the amino acids from the N-terminal or the 14th position to the 685th position amino acid.

In the present application, the term “RBD” refers to the receptor-binding domain of the SARS-COV-2 spike protein (S protein). In the present disclosure, the RBD may be a peptide segment with the amino acids 614-835 of the SARS-COV-2 spike protein (S protein), or a variant thereof, and it may also be a peptide segment obtained by truncating 1, 2, 3, 4, 5, 6, 7, 8, 9, 10, 11, 12, 13, 14, 15, 16, 17, 20, 25 or 30 amino acids at the N-terminal or C-terminal. In the present disclosure, the RBD may be a peptide segment with the amino acids 836-854 of the SARS-COV-2 spike protein (S protein), or a variant thereof, and it may also be a peptide segment obtained by truncating 1, 2, 3, 4, 5, 6, 7, 8, 9, 10, 11, 12, 13, 14, 15, 16, 17, 20, 25 or 30 amino acids at the N-terminal or C-terminal. In the present disclosure, the RBD may be a peptide segment with the amino acids 310-560 of the SARS-COV-2 spike protein (S protein), or a variant thereof, and it may also be a peptide segment obtained by truncating 1, 2, 3, 4, 5, 6, 7, 8, 9, 10, 11, 12, 13, 14, 15, 16, 17, 20, 25 or 30 amino acids at the N-terminal or C-terminal. In the present disclosure, the said RBD may be a peptide segment with the amino acids 319-541 of the SARS-COV-2 spike protein (S protein), or a variant thereof, and it may also be a peptide segment obtained by truncating the amino acids 1, 2, 3, 4, 5, 6, 7, 8, 9, 10, 11, 12, 13, 14, 15, 16, 17, 20, 25 or 30 at the N-terminal or C-terminal as appropriate. In the present disclosure, the said RBD may be a peptide segment with the amino acids 331-524 of the SARS-COV-2 spike protein (S protein), or a variant thereof, and it may also be a peptide segment obtained by truncating 1, 2, 3, 4, 5, 6, 7, 8, 9, 10, 11, 12, 13, 14, 15, 16, 17, 20, 25 or 30 amino acids at the N-terminal or C-terminal as appropriate. In the present application, the term “variant” generally refers to a sequence that differs from the reference sequence by containing one or more differences (mutations). This difference may be a substitution, deletion, or insertion of one or more amino acids. In some embodiments, said RBD may comprise the RBD of SARS-COV-2 (Gamma variant). In some embodiments, said RBD may comprise the RBD of SARS-COV-2 (Beta variant). In some embodiments, said RBD may comprise the RBD of SARS-COV-2 (Omicron variant).

In the present application, the term “NTD” refers to the domain at the N-terminal of the SARS-COV-2 spike protein (S protein). In the present disclosure, said NTD may be a peptide segment with the amino acids 13-353 of the SARS-COV-2 spike protein (S protein), or a variant thereof, and it may also be a peptide segment obtained by truncating 1, 2, 3, 4, 5, 6, 7, 8, 9, 10, 11, 12, 13, 14, 15, 16, 17, 20, 25 or 30 amino acids at the N-terminal or C-terminal as appropriate. For example, the NTD may be a peptide segment with amino acids 13-303 of the SARS-COV-2 spike protein (S protein), or a variant thereof. In the present application, the said NTD may be a peptide segment with amino acids 14-304 of SARS-COV-2 spike protein (S protein), or a variant thereof. In the present application, said NTD may be a peptide segment with amino acids 18-353 of the SARS-COV-2 spike protein (S protein), or a variant thereof.

In some embodiments, said NTD may comprise the NTD of SARS-COV-2 (Gamma variant). In some embodiments, said NTD may comprise the NTD of SARS-COV-2 (Beta variant). In some embodiments, the NTD may comprise the NTD of SARS-COV-2 (Omicron).

In the present application, said variant may comprise any one or several currently known SAR-COV-2 variants (mutations). In the present application, the term “approximately” generally refers to the variation that changes within the range of 0.5% to 10% above or below a specified value, such as the variations within the ranges of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below a specified value.

In the present disclosure, the term “variant” generally refers to a sequence that differs from the reference sequence by containing one or more differences (mutations). This difference may be a substitution, deletion, or insertion of one or more amino acids. In the present disclosure, a protein or a functionally active fragment of a protein generally refers to a protein or a fragment of the protein with similar biological activity.

### 5.2 Microfluidic Platform for High-Throughput Screening

A microfluidic platform is useful for high-throughput generation, analysis, and on-demand isolation of 3D cell-containing droplets. The technology is capable of isolating aqueous droplets comprise one or more cells. The technology is versatile with respect to cell spheroid and droplet size and cellular or chemical composition. The platform that is useful in the present disclosure provides a microfluidic device that can be fabricated through standard soft photolithography. In one embodiment, a microfluidic device for cultivation, analysis, and/or isolation of cells and/or cell spheroids can have two microfabricated layers, each having a network of microfluidic channels, chambers, and other structures, wherein the two layers are interconnected at one or more points. In one embodiment, a first layer can carry out the formation, cultivation, and analysis of cell spheroids, in a microarray of microchambers arranged in a two-dimensional rectangular array of rows and columns. The first layer can include any or all of the following: one or more first inlets, one or more second inlets for an aqueous suspension of cells. The inlets can be further connected to first, second, and third microchannels. The first layer can include a nozzle formed by a T-shaped intersection of two or more of the first, second, and third microchannels, and an incubation chamber containing a plurality of microchambers or docking sites configured in a two-dimensional array of any desired geometry or arrangement of the microchambers/docking sites. The nozzle is capable of producing aqueous droplets. The incubation chamber is fluidically connected to the nozzle and is capable of accepting and delivering the aqueous droplets individually into microchambers. The incubation chamber can comprise one or more windows for monitoring one or more of the microchambers by various means including light microscopy, including fluorescence microscopy, which can provide quantitative analysis or imaging analysis using a photodetector or camera. The droplets then can flow into an array of docking sites or microchambers where cell culture can be performed and observed, for example by light microscopy, or analyzed for the presence or amount of a detectable label such as a dye that absorbs light of a certain wavelength or that exhibits fluorescence.

Methods for the high-throughput screening of cell-cell fusion utilizing droplet microfluidics system and FACS sorter is more quantitative using NGS. Throughput for genetic screens can process moderate number of spike protein variants and pooled assay allows less variants or perturbations.

Methods for the high-throughput screening of cell-cell fusion utilizing size-exclusion selection-based method is more quantitative using NGS. Throughput for genetic screens can process high number of spike protein variants. Genetic variant and perturbation libraries are pooled assembled and delivered into cells. Only a filtration step using a cell strainer is needed to collect the fused cells, which takes seconds. With a reverse selection approach to collect unfused cells, a genome-wide CRISPR library with 37,722 sgRNAs was screened and it took about 8 hrs to sort the unfused cells. Pooled assay allows head-to-head comparison of the variants/perturbations under the same experimental setting.

### 6. EXAMPLES

### 6.1 a Droplet Microfluidics-Based System for High-Throughput Screening of Syncytium Formation

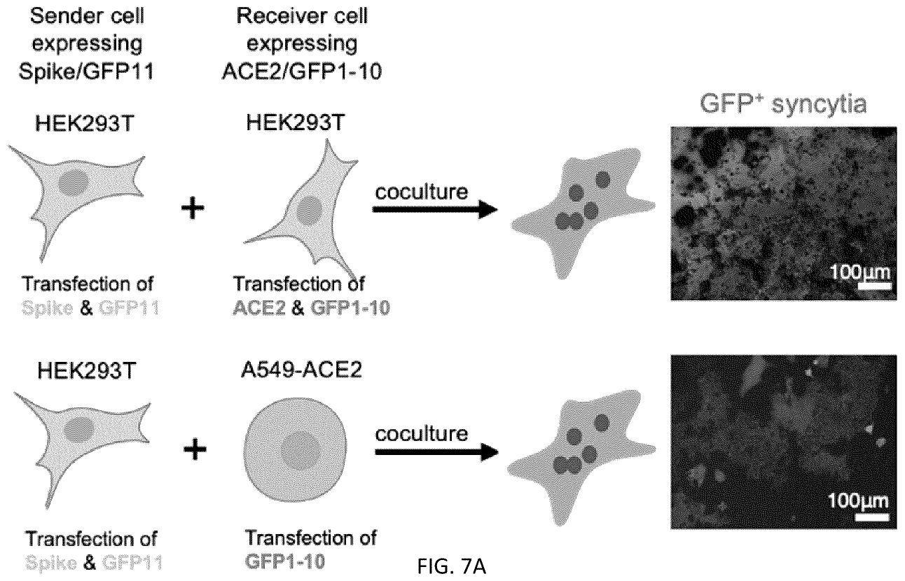

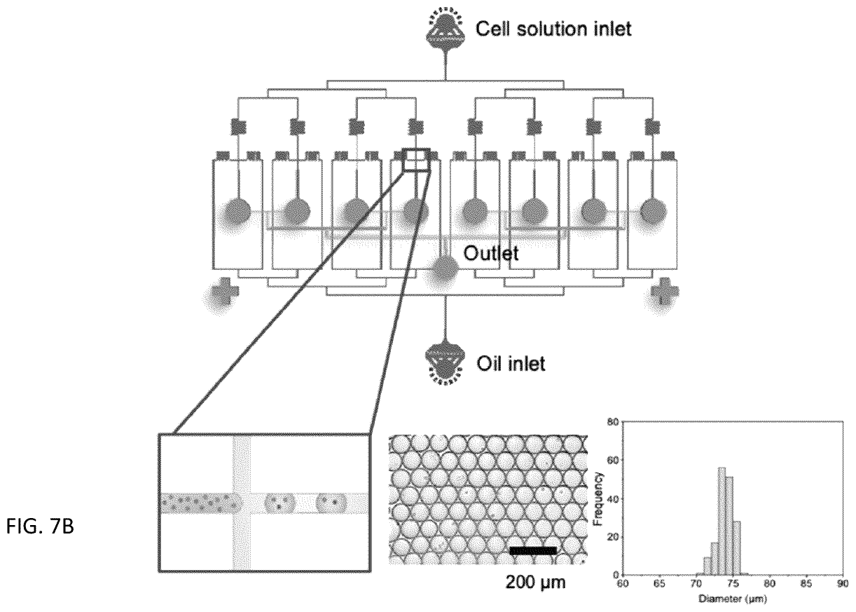

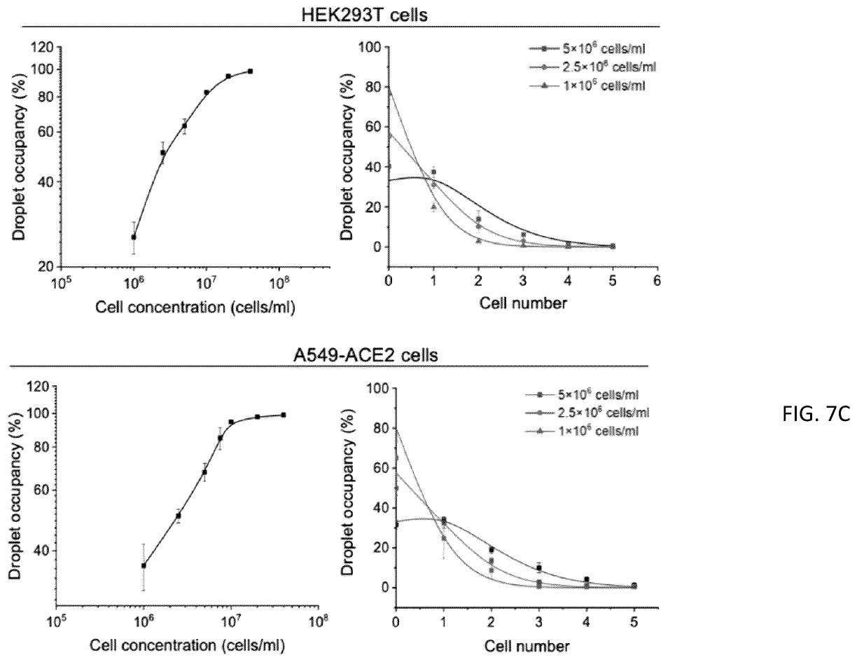

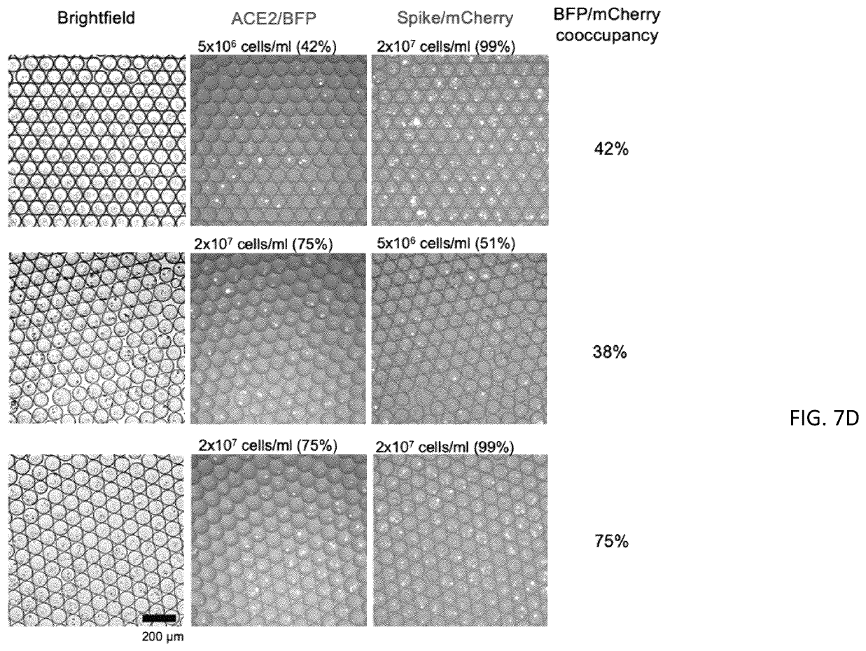

We aimed at establishing high-throughput systems to profile the syncytium-forming potential systematically and quantitatively across sender and receiver cell variants. To visualize cell-cell fusion, we adapted the green fluorescent protein (GFP)-split complementation system wherein the sequence of GFP was split between the tenth and the eleventh B-strand to produce GFP1-10 and GFP11, which are non-fluorescent by themselves; however, the reconstituted GFP becomes fluorescent upon complementation²⁹. By co-culturing the fusogenic sender cells transfected with spike and the small GFP11 fragment, and the receiver cells transfected with ACE2 and the GFP1-10 fragment, the spike-mediated cell-cell fusion can be visualized by the GFP signal (FIG. 7A). To minimize expression variations of the spike protein, GFP1-10 and GFP11 among cells, we created a HEK293T sender cell line stably expressing the spike protein with GFP11-P2A-mCherry, as well as a receiver A549 cell line expressing ACE2, GFP1-10 and blue fluorescent protein (BFP), through lentiviral transductions. A549-ACE2 cells are derived from human lung epithelial cells and are widely used as a model to study SARS-COV-2 biology. The fast-growing nature of A549 is suitable for high-throughput screening experiments. HEK293T cells have no ACE2 expression and were used as the spike-expressing sender cells to avoid cell fusion in the stable cell line, as a low level of ACE2 was detected in A549 cells30. After co-culturing the sender and receiver cells for 24 h, we observed obvious syncytium formation (FIG. 7A).

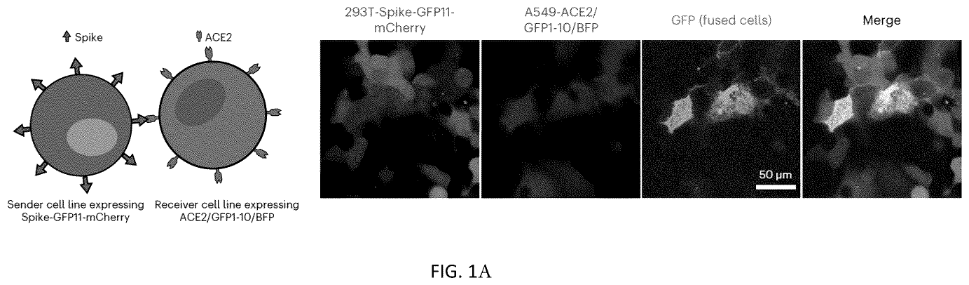

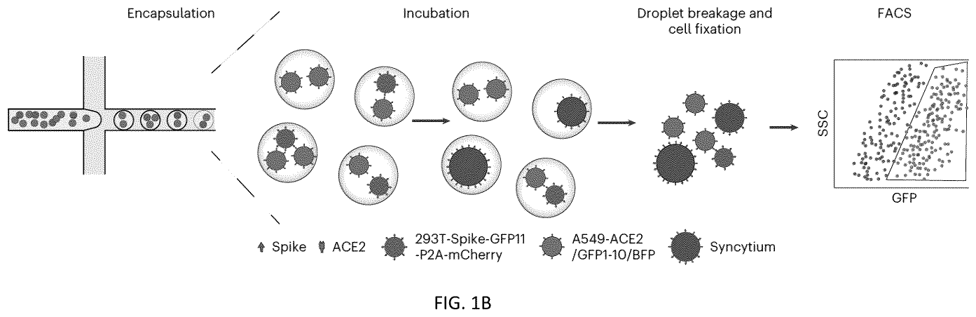

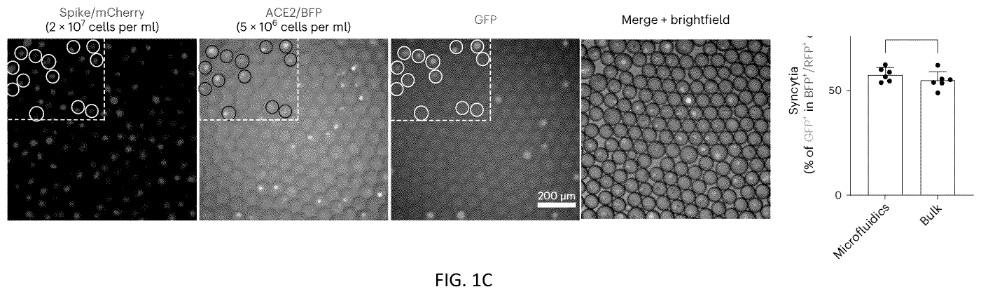

To enable parallel profiling of the syncytium-forming potential of cell variants, we set up a microfluidic system to compartmentalize individual sender and receiver cells in droplets. Although the droplet generation frequency with a standard single-channel device can be as high as 1.5 kHz, the generation time could last as long as 8 h for generating 42 millions of droplets needed for a screening experiment of ˜1,000 variants. Such a long duration of microfluidic processing is not desired as it reduces cell viability. Therefore, we designed an 8-channel device to increase the throughput (FIG. 7B). In comparison, the 8-channel device can maintain an ultrahigh overall frequency of 12 kHz to complete the droplet generation process in ˜1 h. To minimize the uneven distribution of the contents of the single-cell and oil inlets across the eight drop makers, we used a symmetrical layout for the 8 channels such that the flow rates of the aqueous and oil phases can be evenly distributed in each channel. We optimized the flow rates of two phases to generate droplets for encapsulating human cells. In our experiments, we observed droplets with diameters ranging from 70-76 μm (FIG. 7B). The coefficient of variation (c.v.) was 1.5%, which is on par with other microfluidics settings that generate droplets with a c.v.<3%. We next determined the droplet (co)occupancy rates when supplied with different concentrations of HEK293T sender cells, A549-ACE2 receiver cells and their mixtures (FIGS. 7C and D). For the mixtures, we set the concentration of one cell type to undergo variant screening (that is, sender or receiver) as the limiting factor and ensured that most droplets contained only a single cell of that type. After cell encapsulation and incubation, the droplets were demulsified and collected for cell fixation. GFP-positive syncytia were analyzed and sorted out by fluorescence-activated cell sorting (FACS) (FIG. 1B). With this system and experimental setup, we tested and fixed the incubation time at 24 h in subsequent experiments, when a cell-cell fusion rate of 57.9±3.3% was achieved in the droplets encapsulated with both mCherry-positive sender and BFP-positive receiver cells to enable our assay to detect both fusion-enhancing and inhibiting variants (FIG. 1C). The cell-cell fusion rate in droplets is comparable to what we observed in the bulk setting (that is, without using droplet microfluidics) (FIG. 1C).

### 6.2 Deep Mutational Scan (DMS) of the Syncytium-Forming Potential of SARS-COV-2 Spike Variants

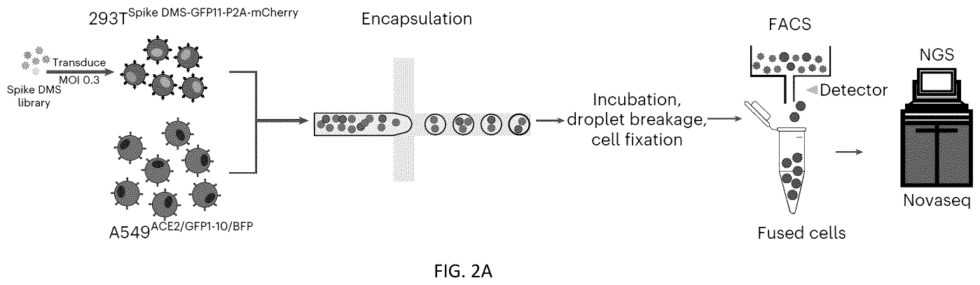

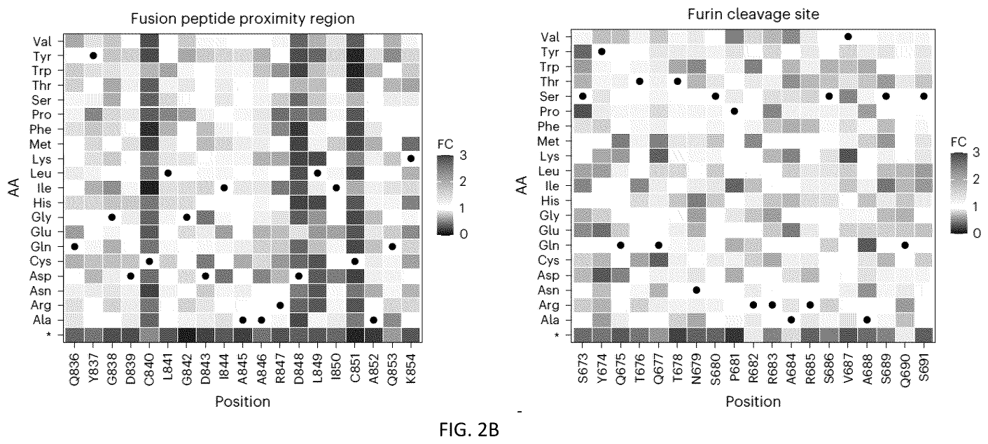

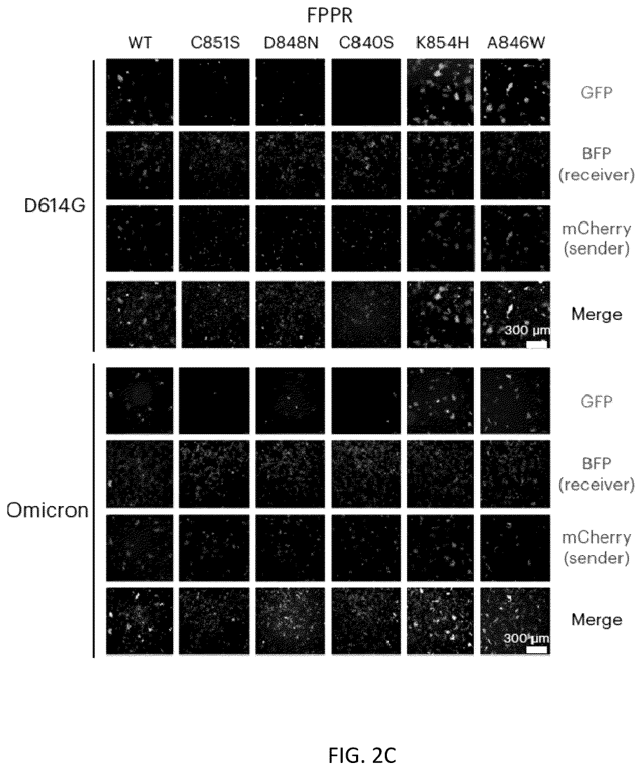

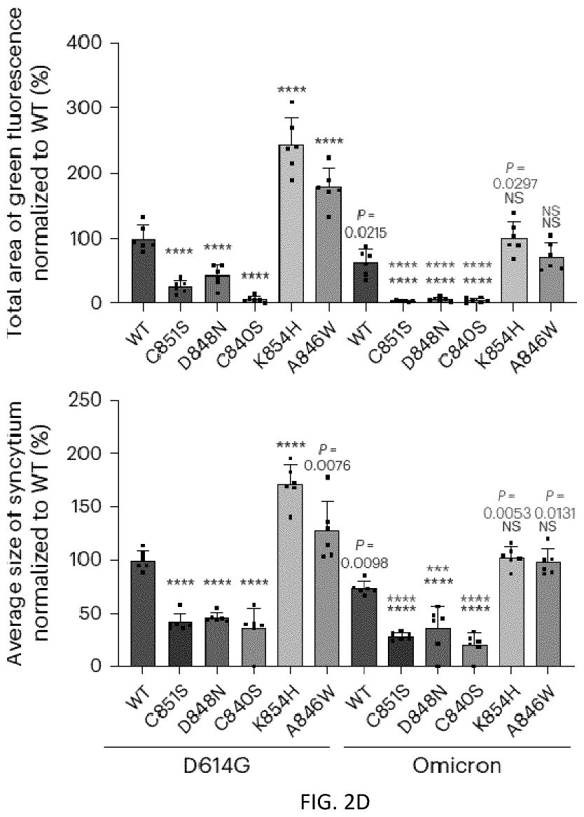

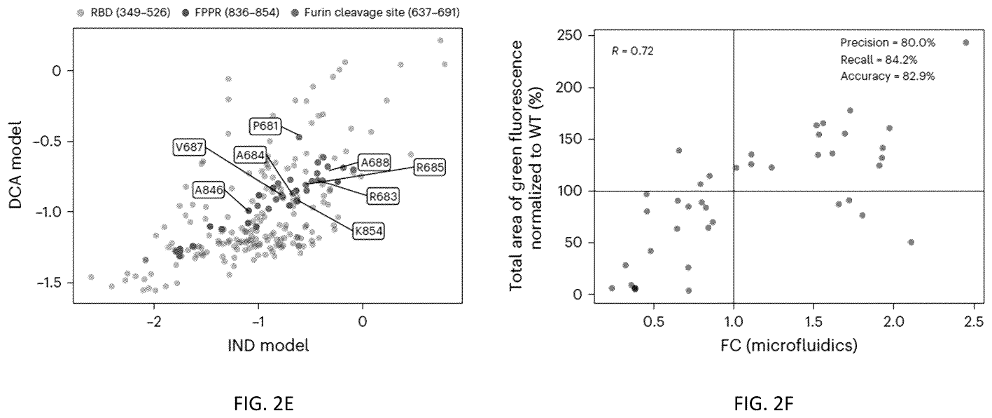

With the droplet microfluidic screening system, we profiled the syncytium-forming potential of spike variants en masse to define residues important for SARS-COV-2-induced syncytium formation and understand how mutations observed in current and future SARS-CoV-2 isolates may impact their syncytium-forming abilities (FIG. 2A). We built a pooled saturation mutagenesis library of spike variants (that is, 20 amino acid residues×19 positions from residues 836 to 854) over the fusion peptide proximal region (FPPR) (FIG. 7A). We used the degenerate ‘NNS’ codon to encode all 20 amino acids and also included stop codons as negative references. We chose the FPPR as one of our regions of interest because it is considered relevant to the conformational changes in spike³¹. Also, various mutations at this region have been reported in the COG-UK-ME, a database that has documented SARS-COV-2 mutations that have been detected in the COG-UK genome sequence dataset³² (FIG. 7B). The FPPR is located close to the S1/S2 boundary and the S2′ cleavage site. One proposed model31 is that the flexible FPPR region is occasionally flipped out of position duc to intrinsic protein dynamics, which allows the RBD to sample the up conformation and capture ACE2 on the target host cell membrane. This rearrangement would in turn expose the S2′ cleavage site and potentially also the adjacent S1/S2 site to initiate the cascade of proteolytic cleavage of spike and its structural transition from the prefusion to the post-fusion state, which induces cell mem-brane fusion. We used lentiviruses to deliver the pooled library of the spike variants with GFP11-P2A-mCherry into HEK293T sender cells (FIG. 7C). A multiplicity of infection (MOI) of ˜0.3 was used to ensure that most cells acquired a single copy of the variant. To ensure that the high-coverage library contained a sufficient representation of each variant (>500-fold coverage of each spike variant), we used >1,500-fold more cells for lentiviral infection than the size of the library being tested. To maintain the library distribution throughout the entire screening process, all the mCherry-positive infected sender cells were sorted out, expanded and then co-cultured with receiver A549 cells expressing ACE2, GFP1-10 and BFP for 24 h before droplet breakage and cell fixation. We performed NovaSeq-based sequencing on the genomic DNA from the collected GFP-positive cells to quantify the abundance of each spike variant as an index of syncytium-forming potential (FIG. 7D). We collected the genome DNA from the infected sender cell pool immediately before mixing with the receiver cells for comparison and confirmed that there were no overrepresented variants in the library (FIG. 7C). Spike variants that have increased syncytium-formation potential were enriched (with fold change >1, comparing each variant's relative abundance in the GFP-positive cell pool vs the cell pool before mixing, normalized to WT), while those that have decreased syncytium-formation potential and those that acquired a premature stop codon after mutation were depleted (with fold change <1).

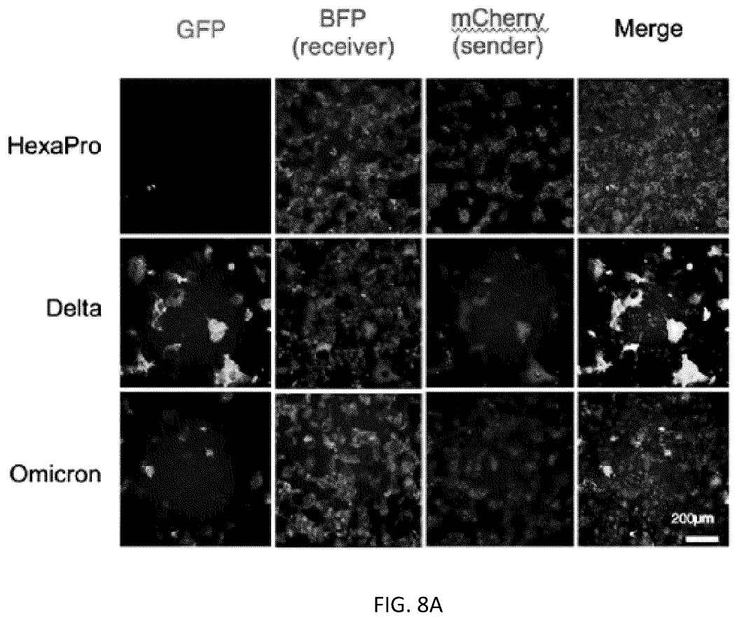

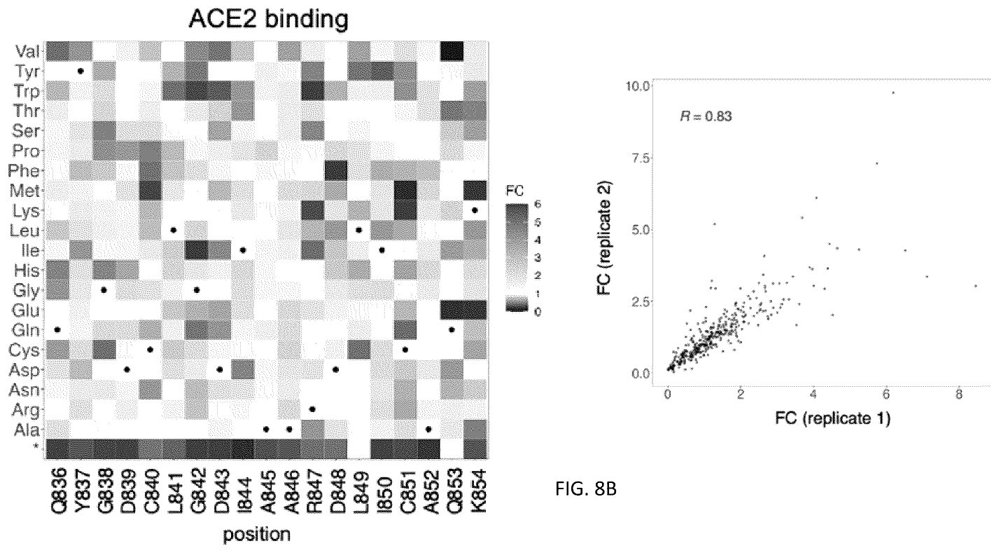

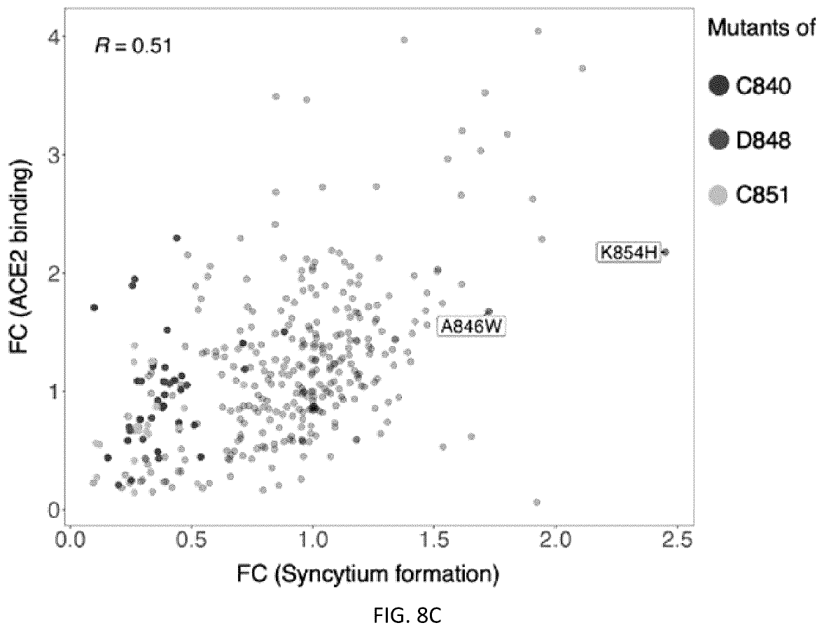

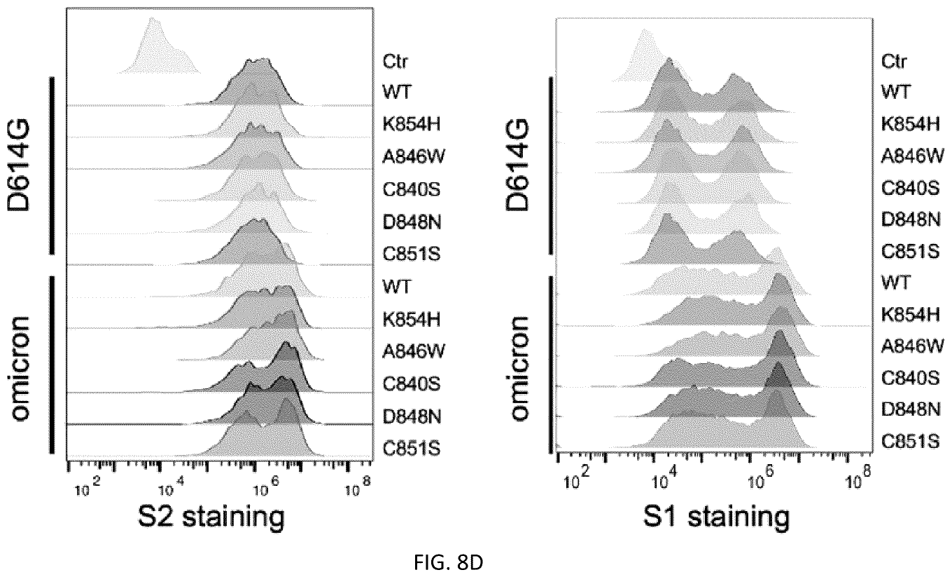

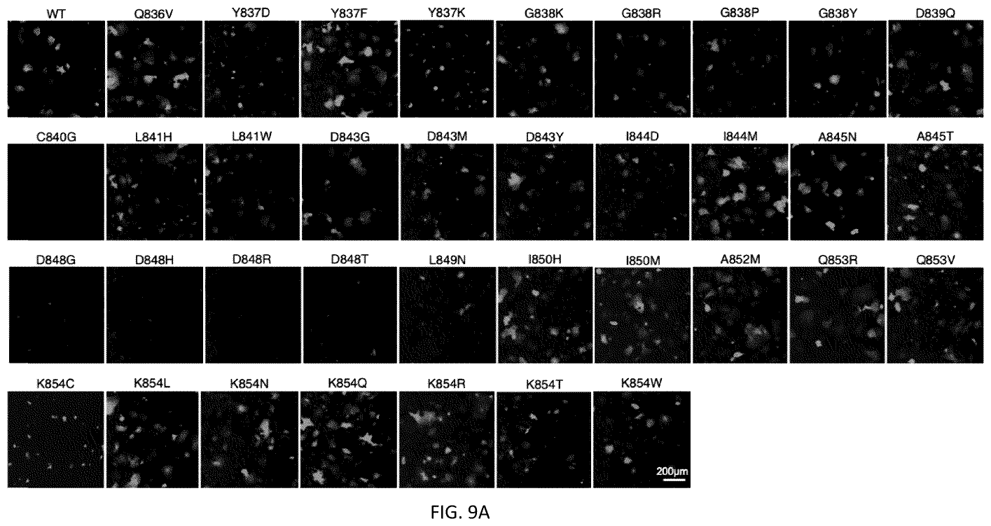

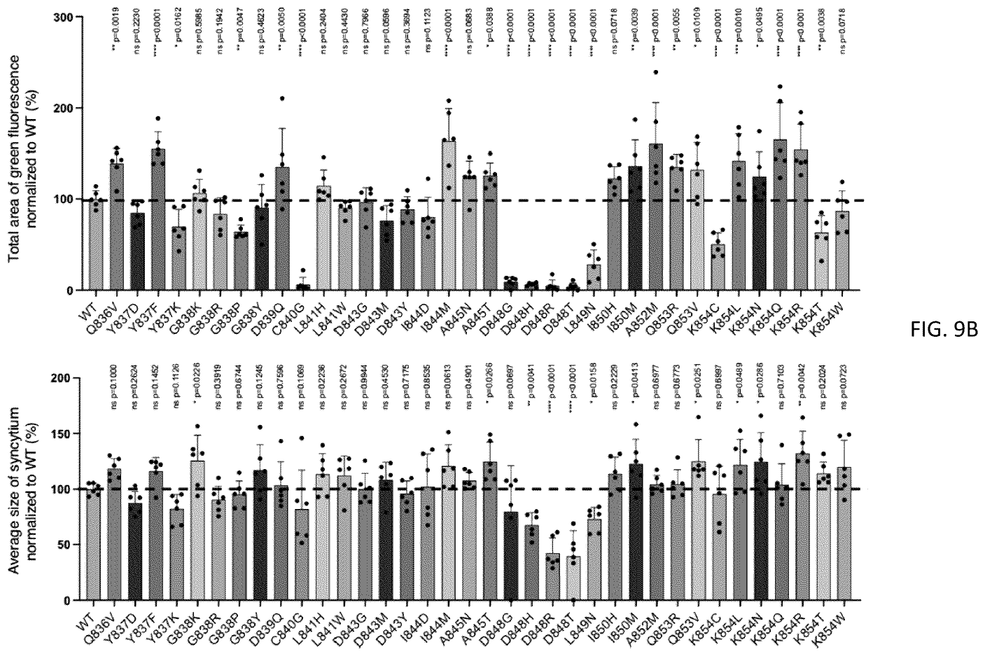

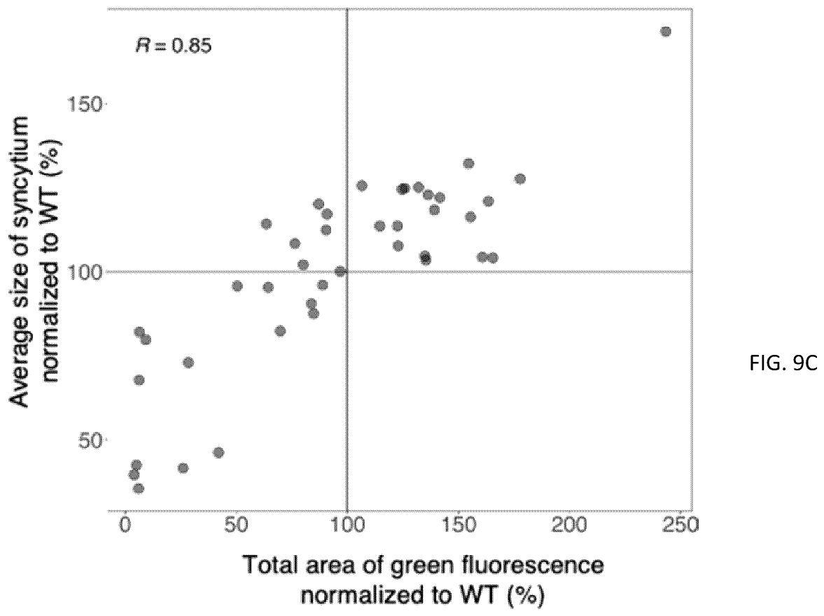

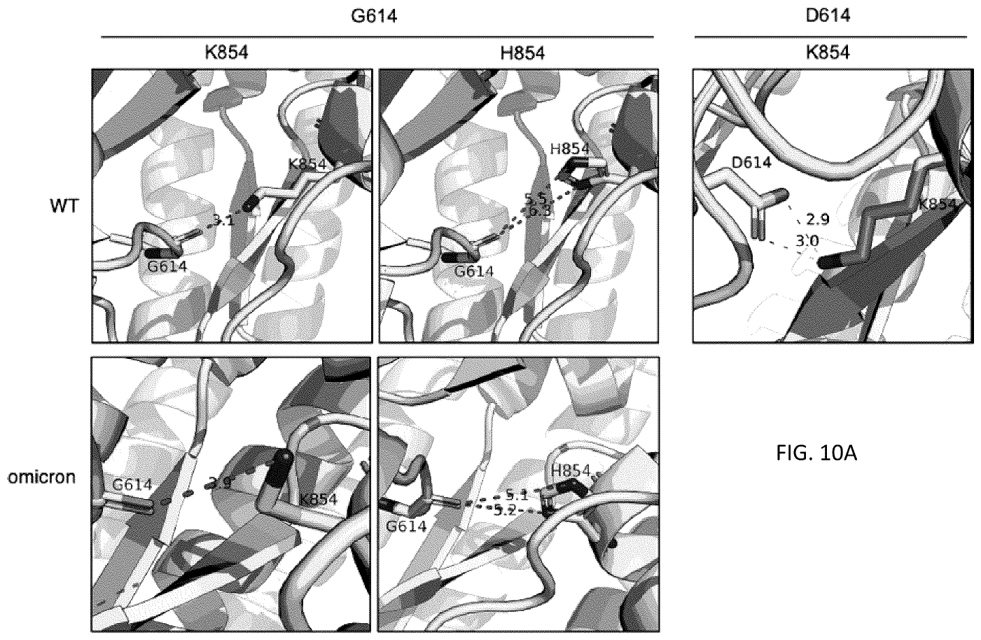

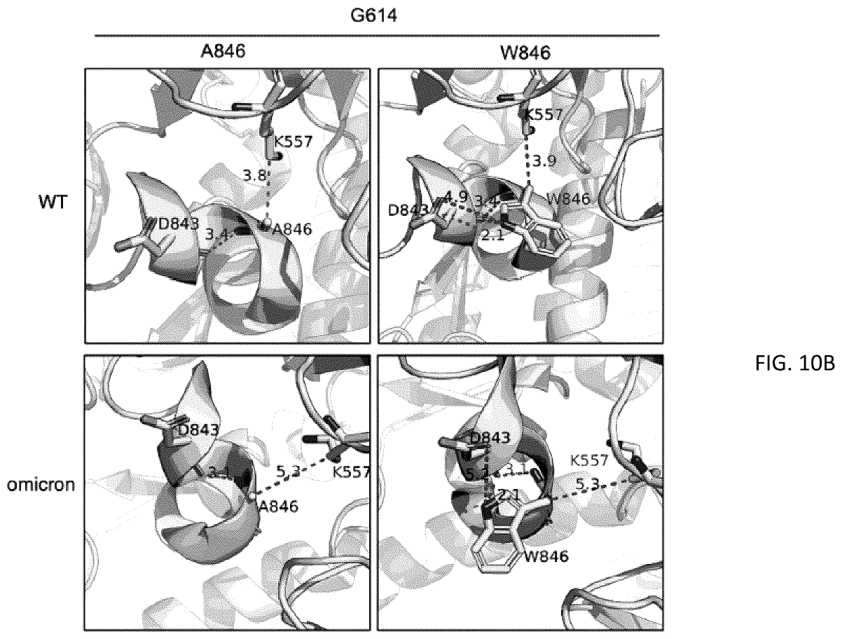

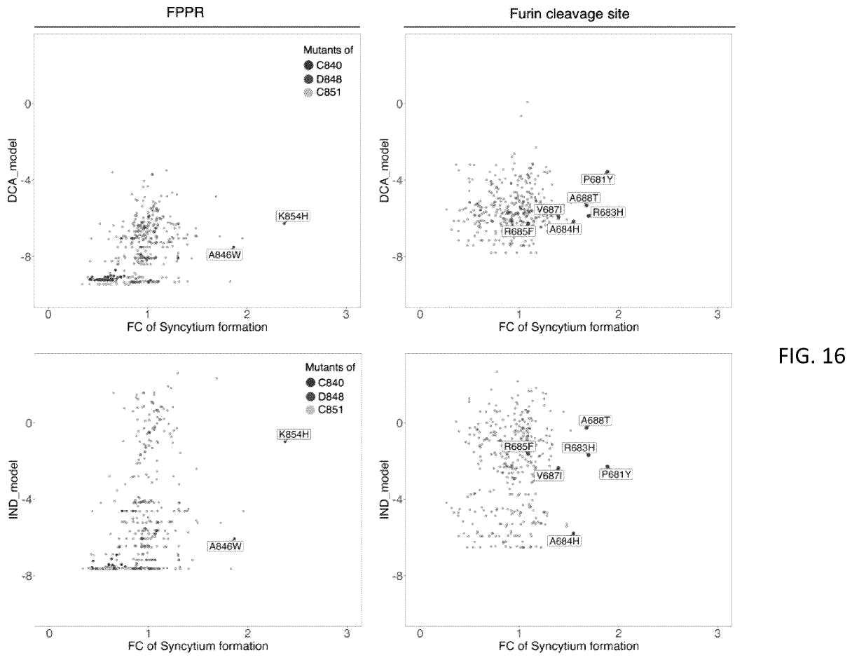

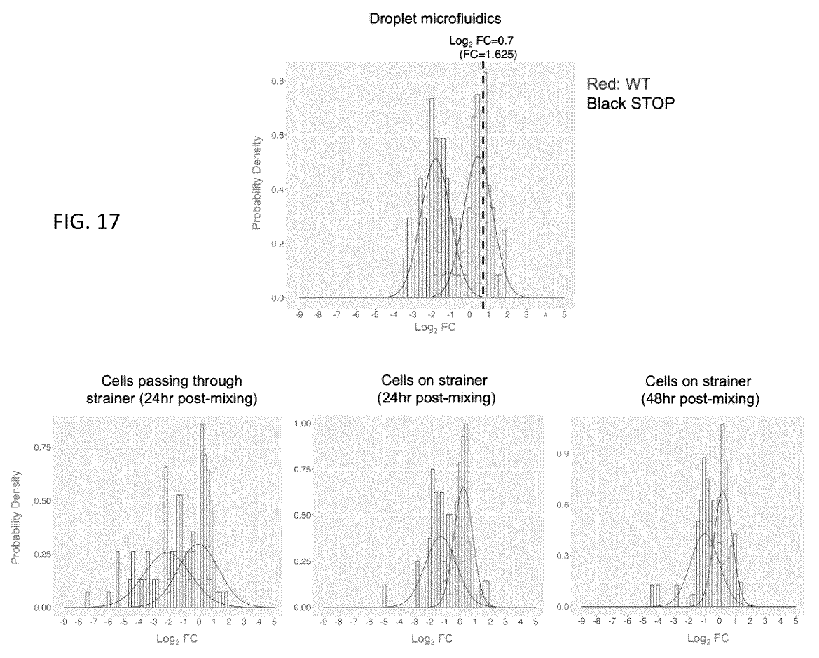

Our profiling result showed that all spike variants with mutations at C840, D848 and C851 tended to have decreased syncytium formation potential (FIG. 2B and Source Data). Individual validation assays confirmed that mutations at C840, D848 and C851 reduced syncytium formation when compared with WT spike (FIGS. 2C and D). Similar effects were also detected when the mutations at C840, D848 and C851 were grafted onto the Omicron variant of spike (FIGS. 8C and D and FIG. 8A), indicating their conserved roles in determining the spike protein's function. These results are in good agreement with the previously reported structural framework of spike, wherein an internal disulfide bond is formed between C840 and C851, which is reinforced by a linked salt bridge that involves D848. This framework was proposed to stabilize the spike protein structure³¹. Interestingly, we found that the mutations at C840, D848 and C851 do not necessarily affect spike's cell surface localization, the cleavage of its S1 subunit and its binding ability with ACE2 (FIGS. 8B and D and Source Data), suggesting that this framework determines spike's syncytium-forming potential independent of these factors. Of note, we also observed spike variants with enhanced ability to form syncytia (FIG. 2B and Source Data) and mutability comparable to other residues at the spike's RBD (FIG. 2E, FIG. 16 and Source Data). Using the observed fold change distributions of WT spike variants with synonymous codons and those with stop codons, we defined a threshold of fold change >1.625 at which no variant with stop codons was identified, thus potentially minimizing the identification of ““““““““false”””””””” false. positives (FIG. 17). With this, 11 syncytium-enhancing hits were identified (Source Data). We performed individual validation assays and confirmed 9 hits exhibiting greater syncytium-forming potential than WT (that is, resulted in larger average size of syncytium and/or total area of syncytia (FIGS. 2C and D and FIGS. 9A and B)). Among them, the K854H and A846W variants showed the greatest enhancement in forming syncytia. K854 has been shown to form a salt bridge with D614 (ref. 31) while the D614G mutation became the first identified SARS-COV-2 isolate that quickly became the predominant form worldwide upon its emergence, and this variant exhibits more efficient cell entry33-35. D614G was reported to weaken its salt bridge formation with K854, which primes the spike protein to adopt a more opened (that is, RBD-up) conformation for ACE2 binding and subsequent membrane fusion, as well as affect the FPPR density between residues 842 and 846 (refs. 31,36-38). Our molecular modelling showed that K854H further decreases its interaction with G614 (FIG. 10A), and the A-to-W substitution at residue 846 increases its interaction with residue 843, which could modulate the FPPR density (FIG. 10B). The mechanisms by which these K854H and A846W mutations enhance syncytium formation remains to be delineated, while they appeared to increase spike's ACE2 binding affinity (FIGS. 8B and C), as reported for D614G39, but not affect its cell surface expression and S1 subunit cleavage (FIG. 8D). Notably, the K854H variant confers Omicron's spike with syncytium-forming potential comparable to that of WT D614G (FIG. 2C, D). In sum, our results reveal that syncytium-enhancing mutations such as K854H exist at FPPR and should be monitored, as they may be observed and emerge in future viral isolates.

To evaluate the data quality of our screen more comprehensively, we analyzed how the screen read counts of variants correlate with their syncytium-forming potency. In addition to the above 14 validated variants, we randomly picked 27 variants in the FPPR library and validated their syncytium-forming potentials using individual assays (FIGS. 9A and B). Overall, we observed a good correlation (R=0.85) between the quantified average size of the formed syncytium and total syncytium area (FIG. 9C). We also observed a high consistency (R=0.72) between the screen and individual validation results (FIG. 2F), highlighting that our screen provides quantitative measurements of the variants' syncytium-forming potentials reasonably well. Among the total of 41 variants, 19 were found to be syncytium-enhancing in the individual validation assays. Of the 19 variants, 16 were discovered as syncytium-enhancing ‘hits’ in our screen (FIG. 2F), indicating a high true discovery rate for our system in defining syncytium-enhancing mutations. The screen could also identify syncytium-inhibiting variants, albeit some ‘false positives’ (that is, 4 out of 22 inhibitory variants validated in the individual assays) could be detected (FIG. 2F). The precision, recall and accuracy of our screen were 80.0%, 84.2% and 82.9%, respectively.

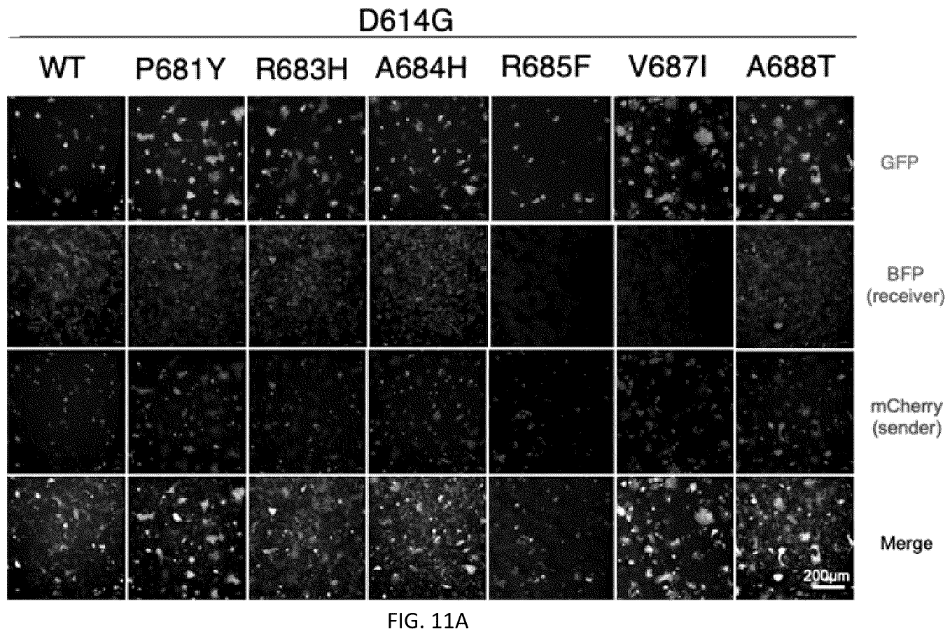

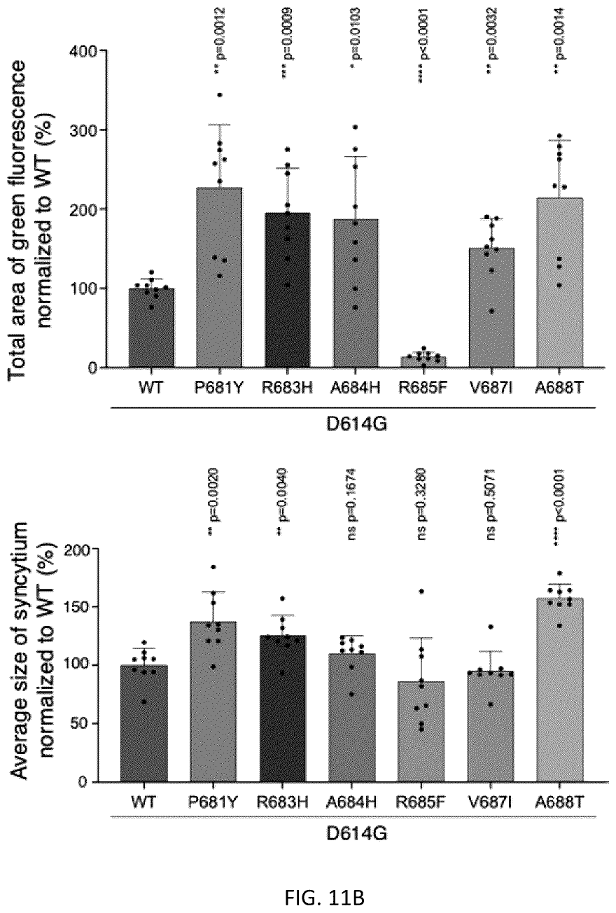

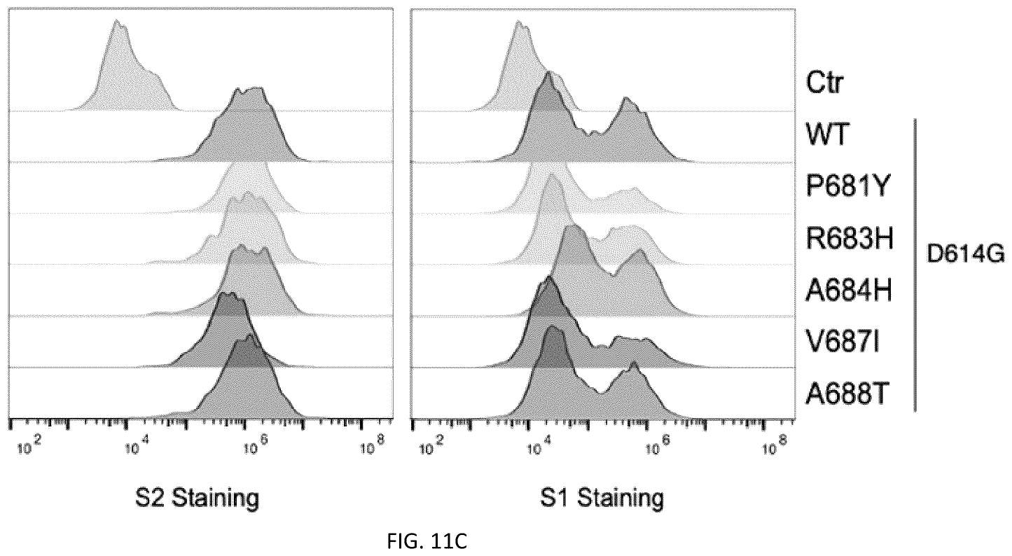

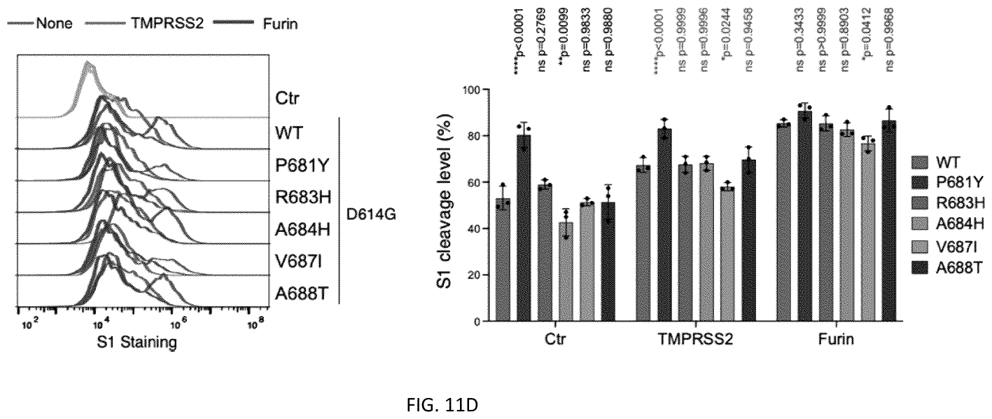

We also applied the droplet microfluidic screening system to examine the region harboring the furin cleavage site in addition to the FPPR. Comparison between the SARS-CoV-2 and SARS-COV-1 protein sequences identified the gain of a short stretch of sequence (that is, PRRA (SEQ ID NO: 1)) before the S1/S2 region in the SARS-COV-2 spike that creates a putative furin cleavage site⁴⁰. It was found that deleting the PRRA sequence (SEQ ID NO: 1) from the SARS-COV-2 spike protein abolishes its ability to form syncytia and viral transmission, while inserting the PRRA sequence (SEQ ID NO: 1) into the SARS-COV-1 spike confers the ability to fuse cells^{5,41}. This finding indicates that PRRA (SEQ ID NO: 1) and potentially its surrounding sequence could be important in the process of membrane fusion leading to syncytium formation by facilitating interactions with and efficient cleavage by proteases. For example, it was reported that furin protease can recognize motif sequences with X-Arg-X-Lys/Arg-Arg-X (that is, XBXBBX, where B is a basic amino acid residue and X is a hydrophobic residue)42. Mutations in this region could impact the syncytium-formation potential of the spike protein. Indeed, P681H or P681R mutation is present in the evolved Alpha, Delta and Omicron variants, indicating the high mutability of this residue. P681R in the Delta variant was shown to slightly increase syncytium formation 13, which was also observed in our deep DMS result, while the increase was at a much lesser extent than another substitution, P681Y (FIG. 2B). P681Y mutation was reported in an immunocompromised patient with persistent SARS-COV-2 Omicron BA.1 subvariant replication43. Here we performed individual validation assays and confirmed P681Y's syncytium-enhancing effect (FIGS. 11A and B), as well as its increased efficiency to be S1-cleaved even in cells with minimal furin and TMPRSS2 expressions (FIGS. 11C and D). Our DMS results further revealed that most single mutations at PRRA (SEQ ID NO: 1) and its neighboring sequence including the basic residue R685 were not sufficient in abolishing syncytium formation (FIG. 2B). These suggest that despite the high mutability of these sites, many of the single mutants can be efficiently cleaved by the proteases. This could be attributed to the rather flexible motif sequence that the furin protease or other proteases could recognize. Our results isolated several syncytium-enhancing and mutable variants that are present in this region (FIGS. 2B and E and FIG. 16). At the fold change cut-off of >1.625 as defined in the FPPR screen, 5 syncytium-enhancing hits were identified (Source Data) and 4 of them (that is, R683H, A684H, V6871 and A688T) were validated with individual assays showing larger average size of syncytia and/or total area of syncytia (FIGS. 11A and B). These mutations did not affect the spike's cell surface expression level and S1 subunit cleavage (FIGS. 11C and D). The potential emergence of P681Y, as well as the other identified syncytium-enhancing mutations at residues 683, 684, 687 and 688 that have similar mutability scores (FIG. 2E and FIG. 8) should be monitored.

Overall, our results validated the utility of the droplet microfluidics-based screening approach to profile the syncytium-formation potential of spike variants.

### 6.3 a Size-Exclusion Selection-Based Strategy for DMS of Spike's Syncytium-Forming Potential

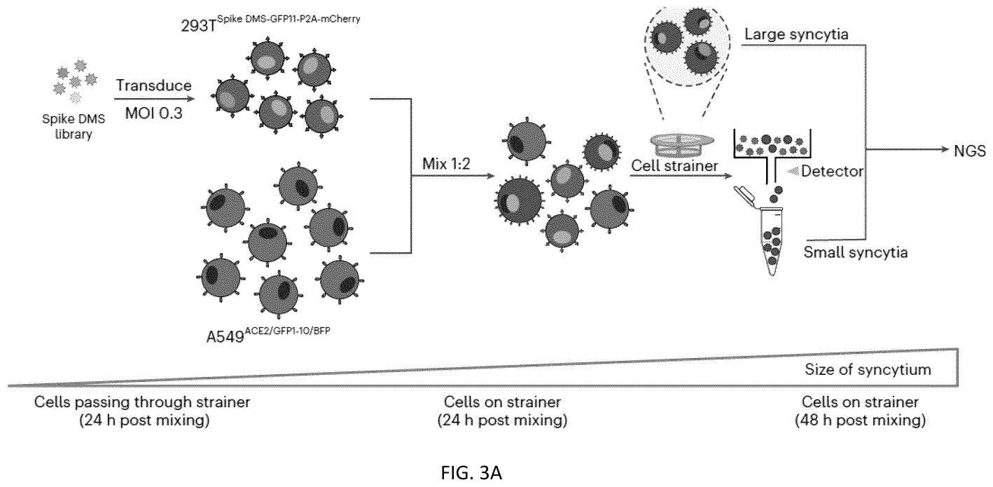

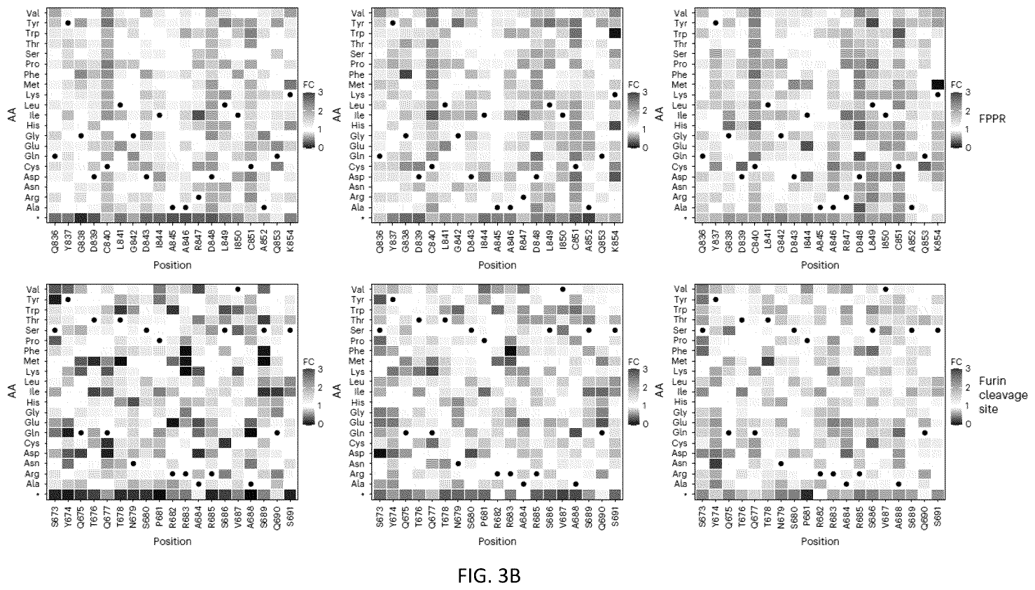

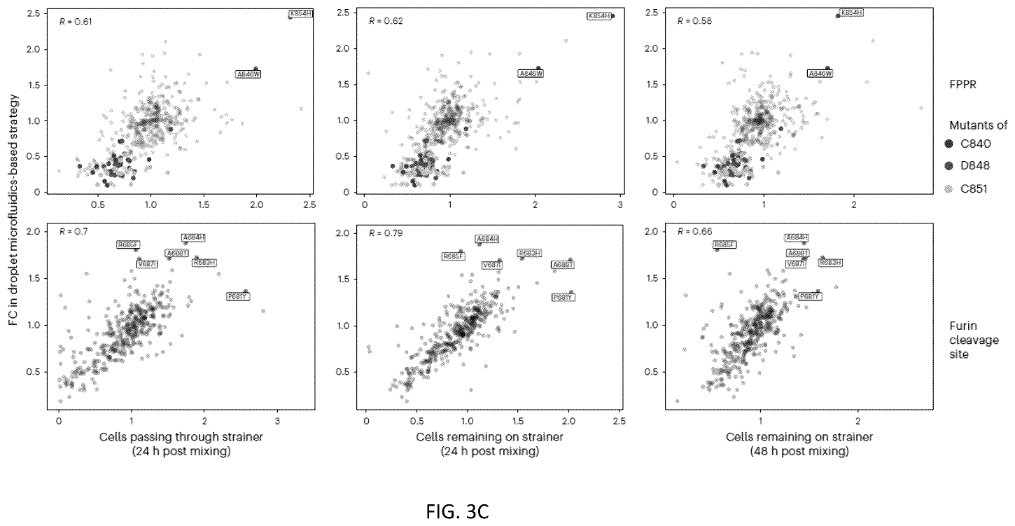

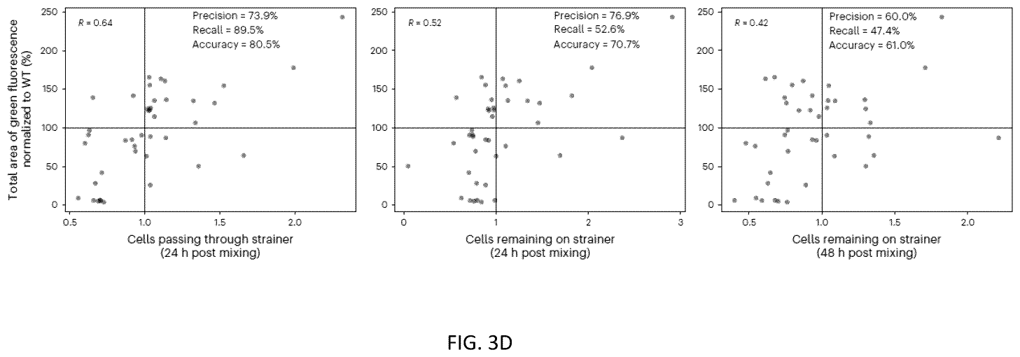

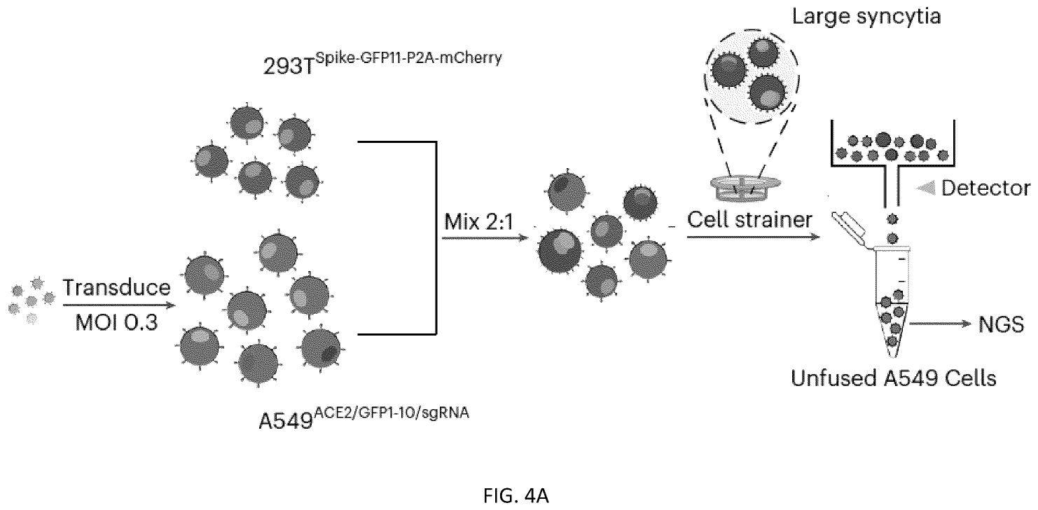

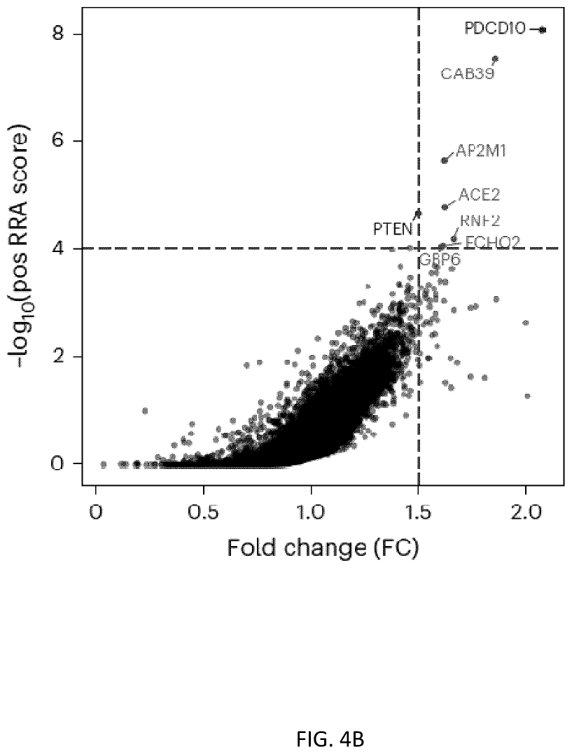

We also explored the feasibility of developing an alternative compartmentation-free selection tactic to enhance the throughput of SARS-COV-2 spike-mediated syncytia screening. Since large syncytia formed in the co-culture system cannot be subjected to flow-cytometry analysis, we attempted to use a 70 μm cell strainer to collect them. At the same time, we also performed FACS to collect all GFP-positive cells that passed through the cell strainer, that is, the small syncytia resulting from fusion (FIG. 3A). Therefore, we separated fused cells from unfused cells to analyze the relative abundance of each variant within a library of cells by sequencing the DMS region encoded in the integrated lentiviral sequence in the genomic DNA (FIG. 3A). Spike variants that have enhanced syncytium-formation potential were enriched in both collected populations of large and small syncytia, while those that have reduced syncytium-formation potential and those that contain a premature stop codon after mutation were depleted. Using this size-exclusion selection-based strategy, we were able to obtain profiling results that largely resemble the data collected from the droplet microfluidics-based system (FIGS. 3B and C and Source Data). Using size-exclusion selection-based strategy could thus offer a simple and scalable way to perform large-scale DMS for syncytia screening.

We evaluated the quality of data collected from the screen via the size-exclusion selection strategy. Among the 19 (out of 41) FPPR library variants with individually validated syncytium-enhancing potentials (FIGS. 9A and C), 17 were discovered as syncytium-enhancing ‘hits’ (with fold change >1) in our screen using the GFP-positive small syncytia collected via FACS (FIG. 3D). This gives a true discovery rate similar to that of the droplet microfluidics-based screening approach. The hit number dropped to 10 and 9 when screening larger syncytia collected and remaining on the cell strainer after 24 and 48 h post mixing, respectively (FIG. 3D), resulting in reduced true discovery rates. Indeed, we noted that the cells-remaining-on-strainer-based strategy resulted in less depletion for the fusion-incompetent cells in the pooled assay, which gives greater noise and a narrower assay range in defining the enriched fusion-competent variants (FIG. 17). The non-compartmented cell pool in the size-exclusion selection-based system and the longer duration allowed for syncytium formation, potentially favoring the trapping of fusion-incompetent cells by the neighboring syncytia as bystanders and their retention on the cell strainer. Some relatively less fusion-competent cells may also be fused with neighboring syncytia containing the more fusion-competent variants and enriched together as large syncytia with polyclonal sender cells. These could account for the relatively less enrichment and likelihood of the true syncytium-enhancing variants (particularly the weaker ones) to be discovered as hits and more non-enhancing variants being isolated as ‘false positives’, thus increasing the false discovery rate. Among the three experimental parameters for the size-exclusion-based selections, allowing a shorter duration (that is, 24 h) to form smaller-sized syncytia, in particular ones that are small enough to be collected by FACS, is recommended.

All in all, selecting either the droplet microfluidics-based or the size-exclusion selection-based screening strategy could depend on the desired sensitivity and throughput of the genetic screen to be performed. Future efforts could explore using droplet microfluidics to achieve cell size measurement and integrate with fluorescence as dual readouts to further enhance the screening data quality.

### 6.4 Genome-Wide CRISPR Screening Identifies Host Factors Required for Spike-Induced Syncytium Formation

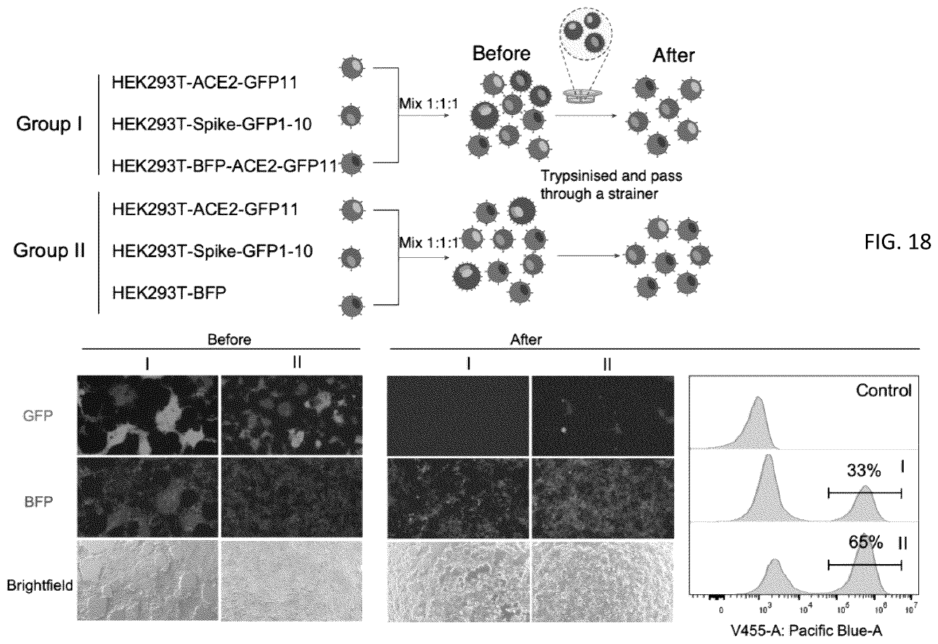

To identify host factors that are required for spike-mediated syncytia, we sought to set up a reciprocal genome-wide CRISPR screen on the basis of the cell-cell fusion system. Because of the large library size needed for genome-wide CRISPR-based knockout screening, we decided to use the size-exclusion selection-based screening strategy. Still, it is technically difficult to directly adopt the selection approach that collects the fused cell population via the cell strainer or the GFP-positive fused cells to look for depleted single guide RNAs (sgRNAs) representing the genes required for cell-cell fusion in the ultra-large pool of cells. Here we took a reverse selection approach to collect all the sgRNA-infected receiver A549-ACE2-Cas9-GFP1-10 cells that are resistant to fusion when co-culturing with the spike-expressing sender cells, with the aim of isolating host factors crucial for the syncytium formation. In our co-culture system, we observed that most of the multinucleated syncytia die or can be removed by a 40 μm cell strainer after prolonged culture (that is, ˜6 d). To confirm this result, we mixed additional HEK293T cells that stably express BFP into the co-culture system, reasoning that if the fused cells die and/or are being removed by the strainer, the percentage of BFP+ cells will increase. We mixed HEK293T-BFP cells, HEK293T-spike-GFP1-10 and HEK293T-ACE2-GFP-11 cells at a ratio of 1:1:1. The percentage of BFP+ cells rose from 33.8% to 60.5% on day 6, which agrees with our reasoning (FIG. 18).

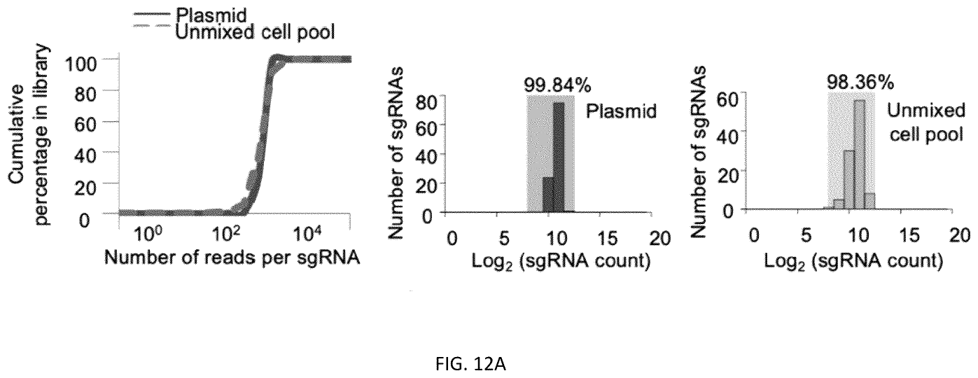

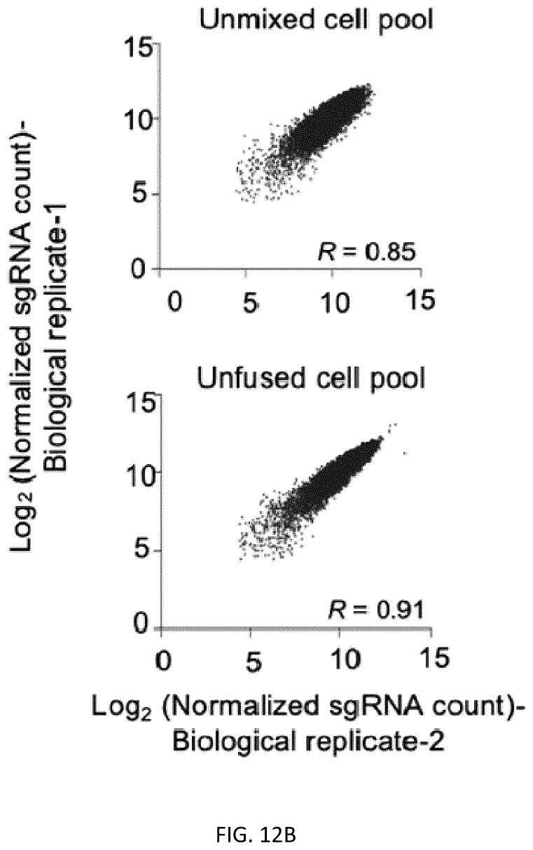

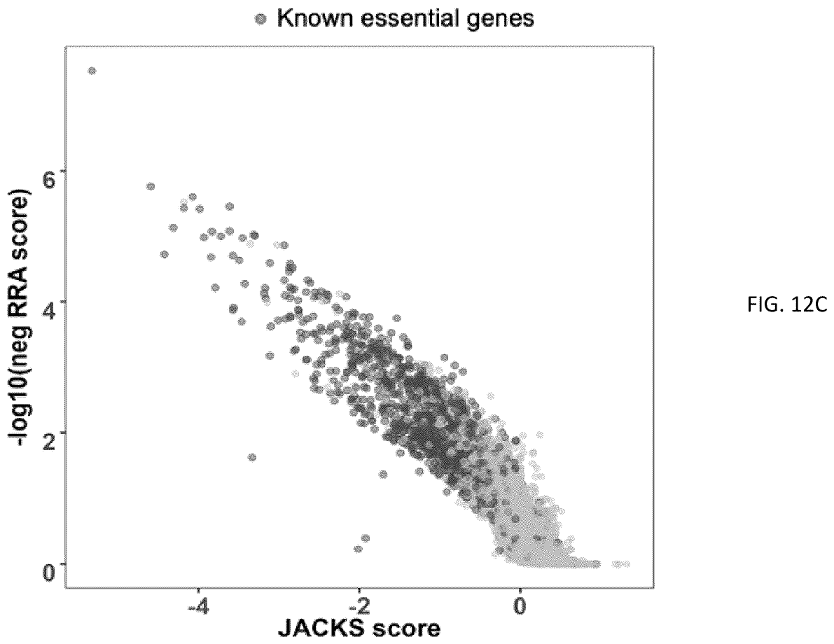

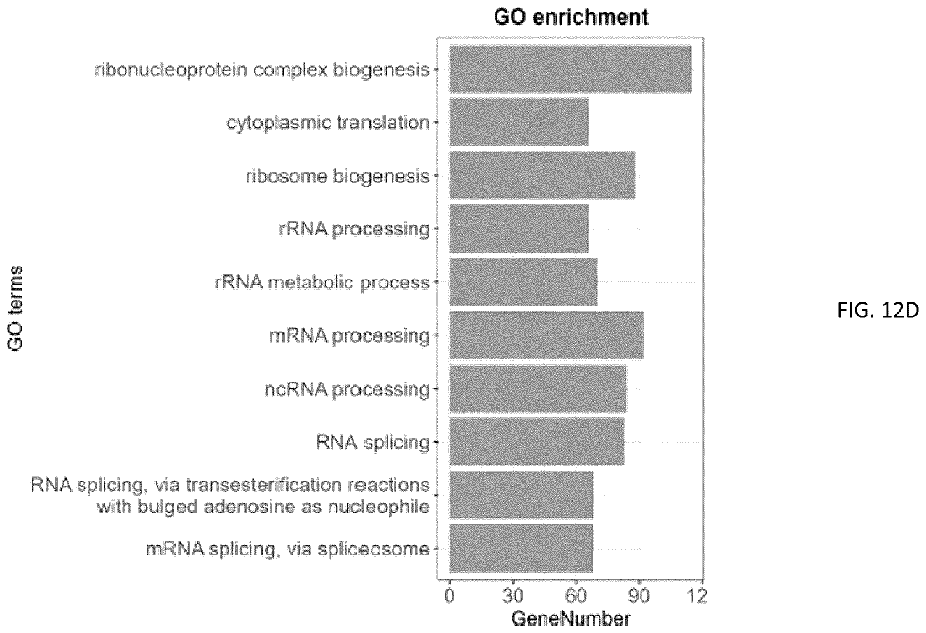

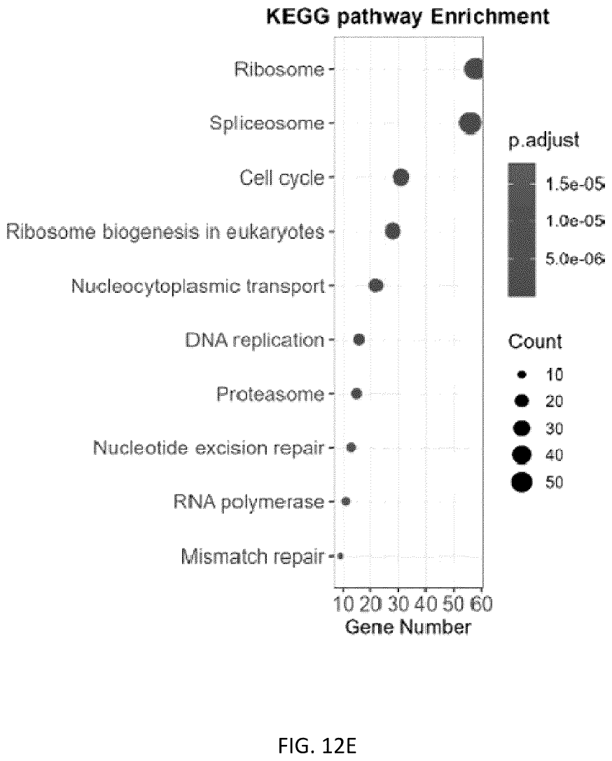

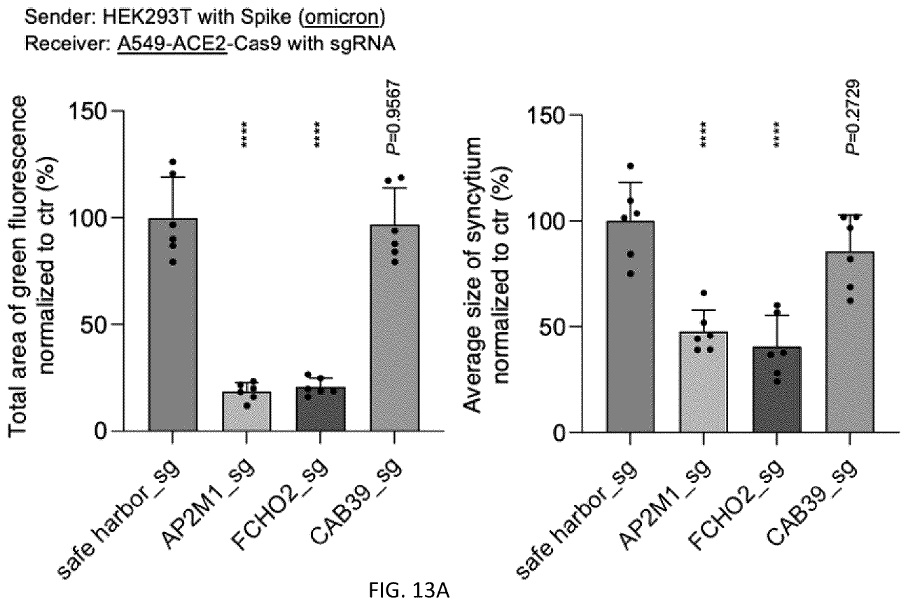

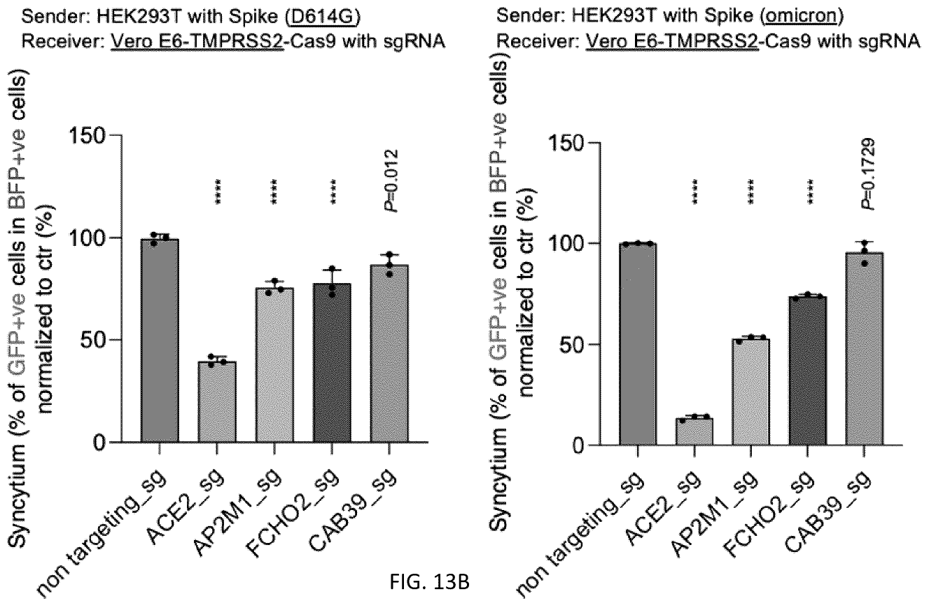

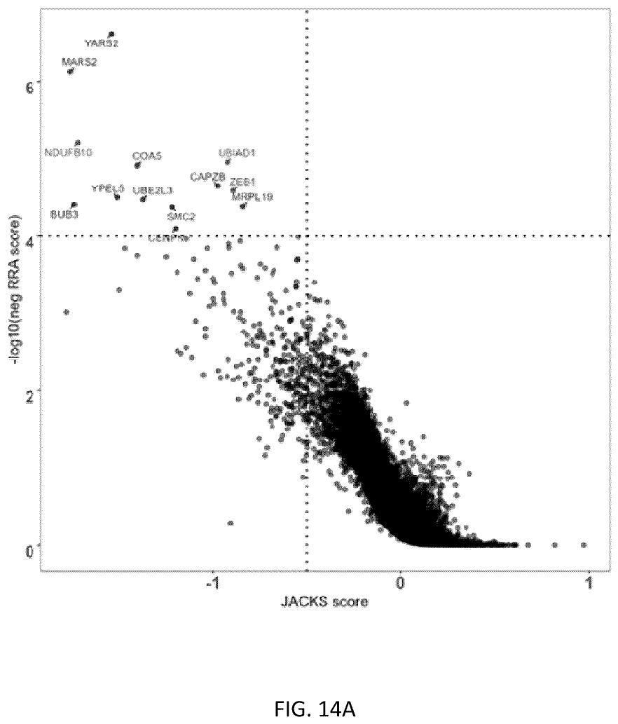

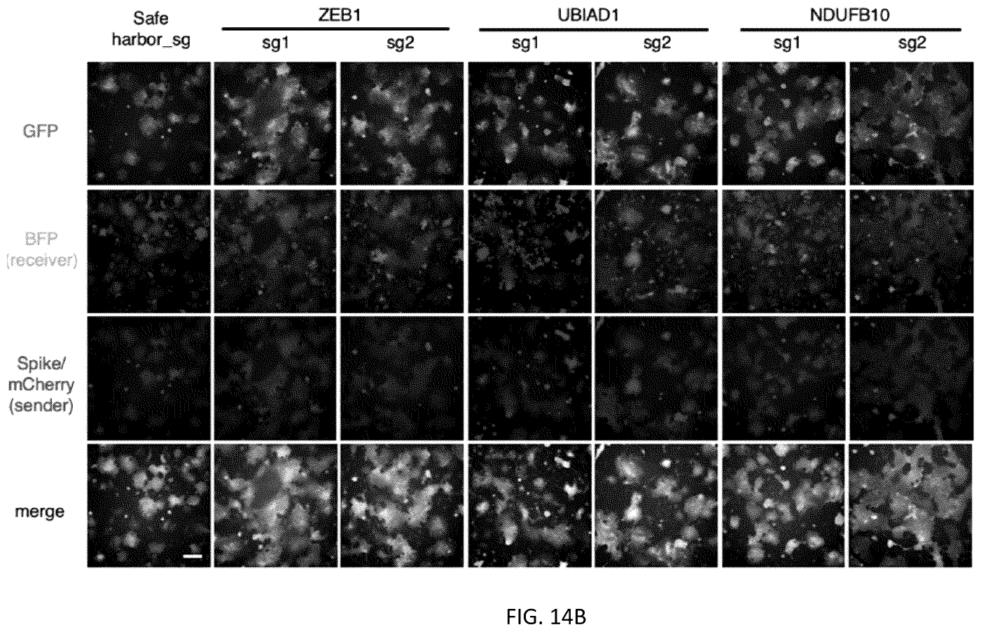

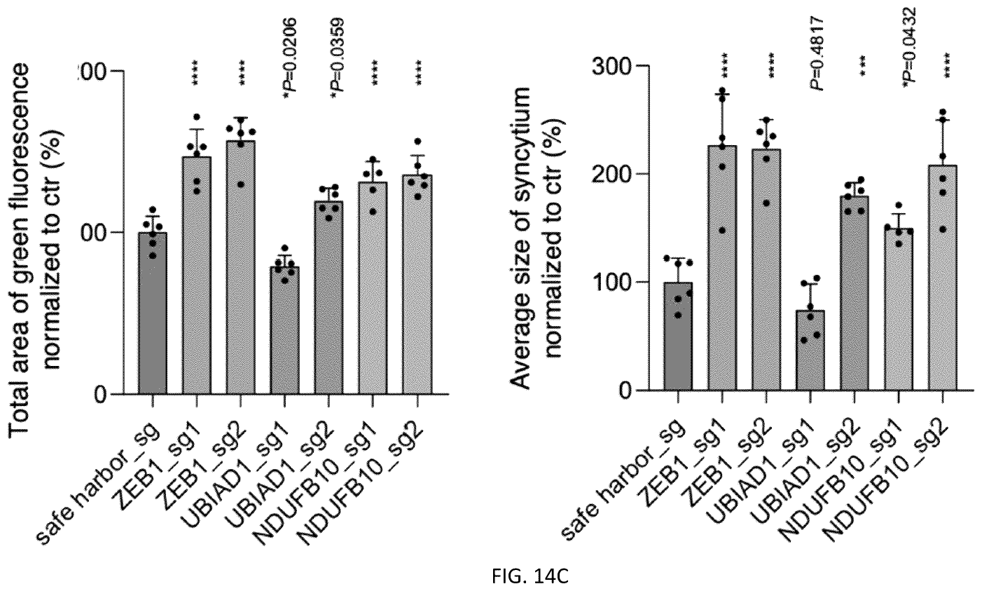

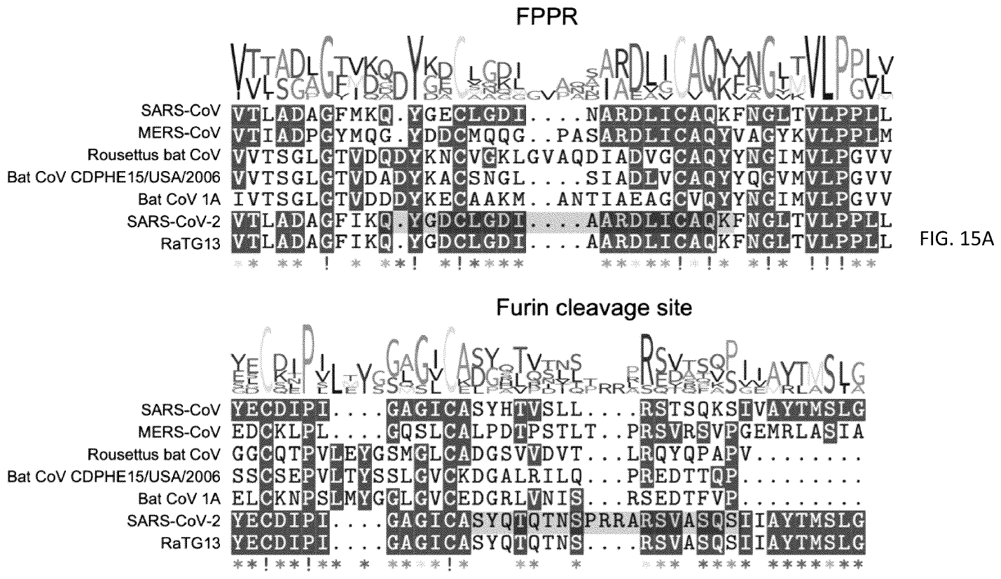

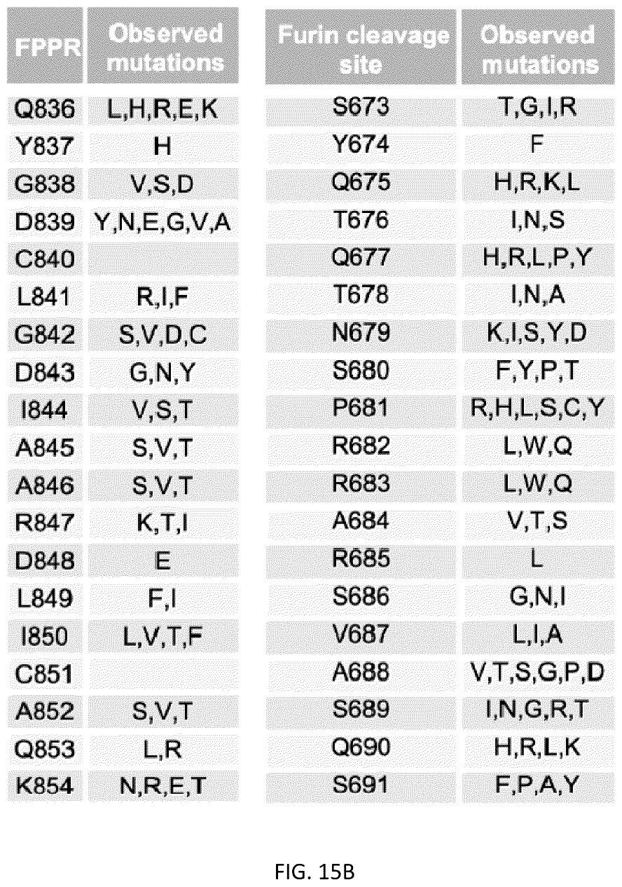

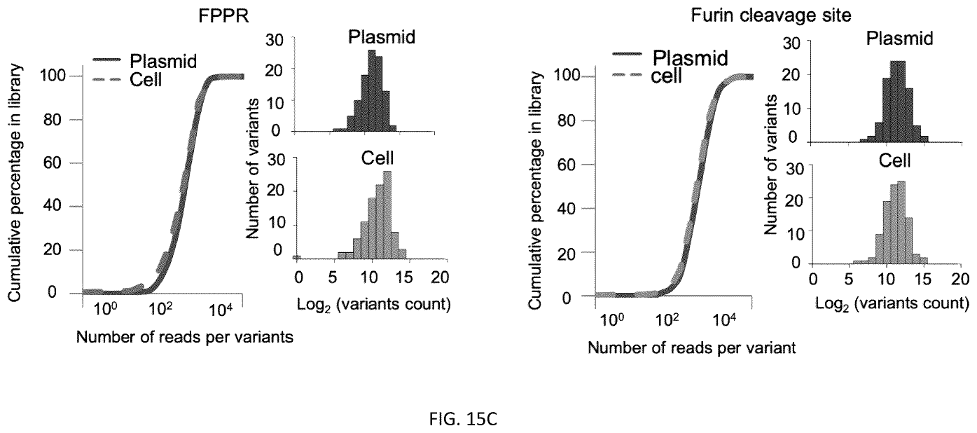

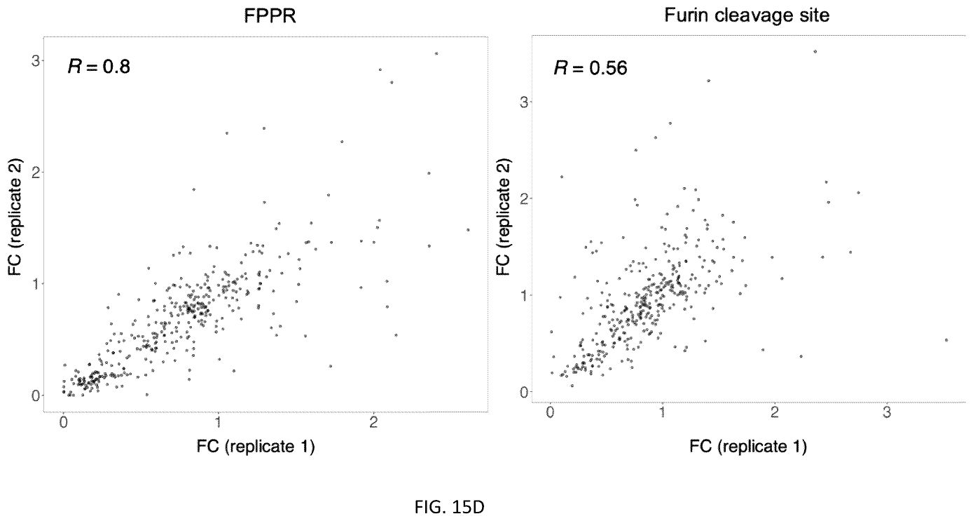

Next, we applied our reverse selection approach to carry out genome-wide CRISPR knockout screening to identify the important host factors for syncytium formation (FIG. 4A). Approximately 150 mil-lion A549-ACE2-SpCas9-GFP1-10 cells were infected with the lentiviral MiniLibCas9 sgRNA library44 at an MOI of ˜0.3 to have each of the 37,722 sgRNAs to be present in >500 unique cells on average (FIG. 12A). On day 6 post infection, the sgRNA-infected cells were sorted out for further culture. After allowing the cells to recover for a week after sorting and expanding in numbers, these A549-ACE2-SpCas9-GFP1-10-sgRNA library cells were mixed with 293T-spike-GFP11-P2A-mCherry cells. At the same time, ˜15 million A549-ACE2-SpCas9-GFP1-10-sgRNA library cells were collected before mixing. The mixed cells were passaged every 3 d. During cell passaging, a 40 μm cell strainer was used to remove the syncytia clumps to enrich the unfused cells. On day 7 post mixing, unfused A549-ACE2-SpCas9-GFP1-10-sgRNA library cells were sorted out for the late-timepoint sample. The genomic DNA of all the samples were extracted and the sgRNA region was PCR amplified for NovaSeq-based sequencing (FIG. 12B). We performed the CRISPR screen in two biological replicates and confirmed the robustness of our library screen in identifying known essential genes (FIG. 12C-E). Upon comparing the sgRNA abundance in unmixed A549-ACE2-SpCas9-GFP1-10-sgRNA library cells and unfused A549-ACE2-SpCas9-GFP1-10-sgRNA library cells using MAGcCK45 and JACKS46, we identified the known ACE2 receptor as well as five other hits (FCHO2, AP2M1, CAB39, RNF2 and GBP6) at arbitrary cut-offs of robust ranking aggregation (RRA) score>4 and fold change >1.5 as potential host factors that play important roles in syncytium formation (FIG. 4B and Source Data). Inhibition of known tumour-suppressors PTEN and PDCD10 is expected to increase cell proliferation, thus these genes were not included in our further analysis. We performed individual validation experiments and confirmed that knockout of the five hits in A549-ACE2 receiver cells inhibited syncytium formation when mixed with the WT spike-expressing sender cells (FIGS. 4C and D). Since knockout of FCHO2 and AP2M1 reduced syncytium formation induced by WT spike at a greater extent than the other three genes (FIGS. 4C and D) and they also inhibited Omicron spike-induced syncytium formation (FIG. 13A), FCHO2 and AP2M1 were thus selected for further characterization. Since A549-ACE2 cells do not express TMPRSS2, our results suggest that the fusion events are not TMPRSS2-mediated. In the absence of TMPRSS2, the spike protein can enter cells through endosomal pathways⁴⁷. We generated FCHO2 and AP2M1 knockouts in Vero E6-TMPRSS2 receiver cells and found that both knockouts inhibited syncytium formation when mixed with the sender cells (FIG. 13B). This result indicates that these two factors are also important for syncytium formation in cells that express TMPRSS2.

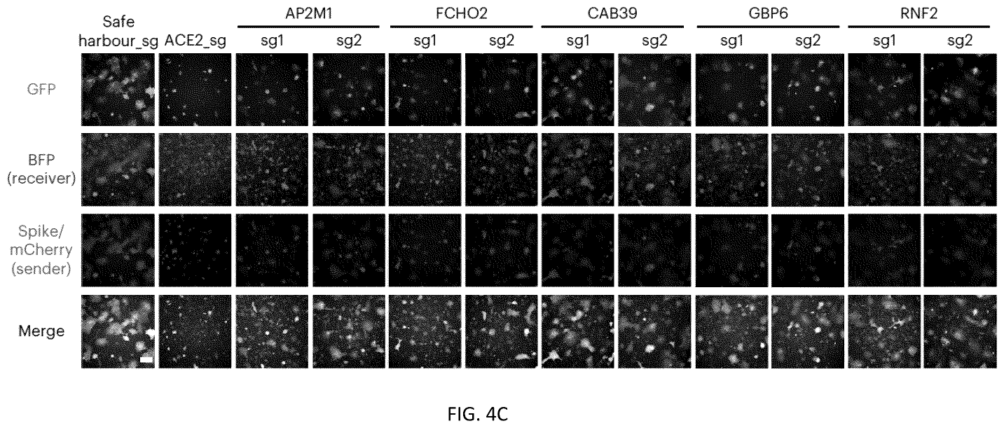

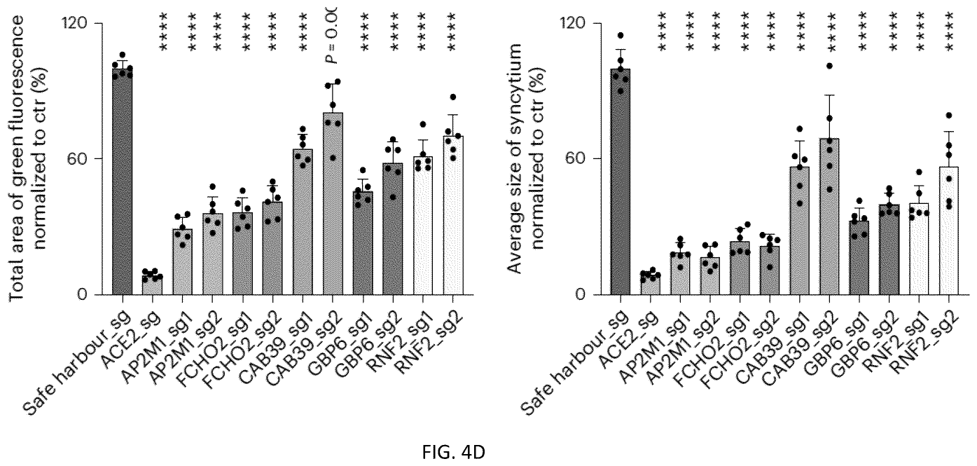

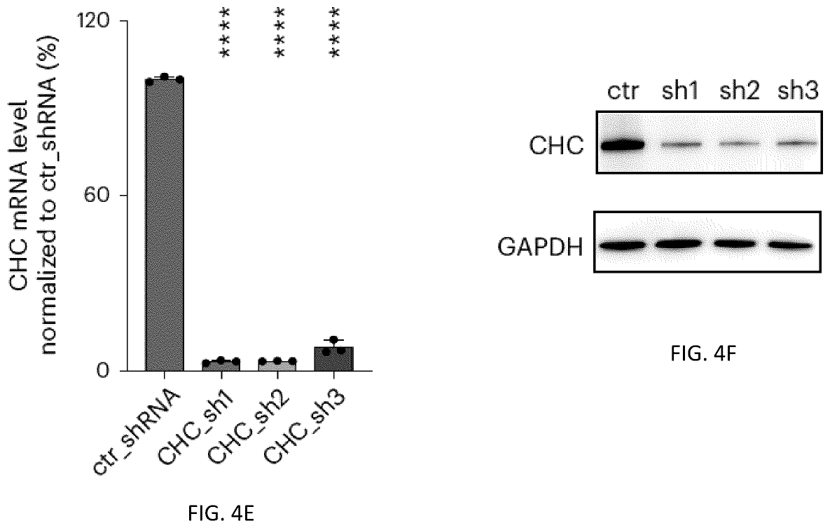

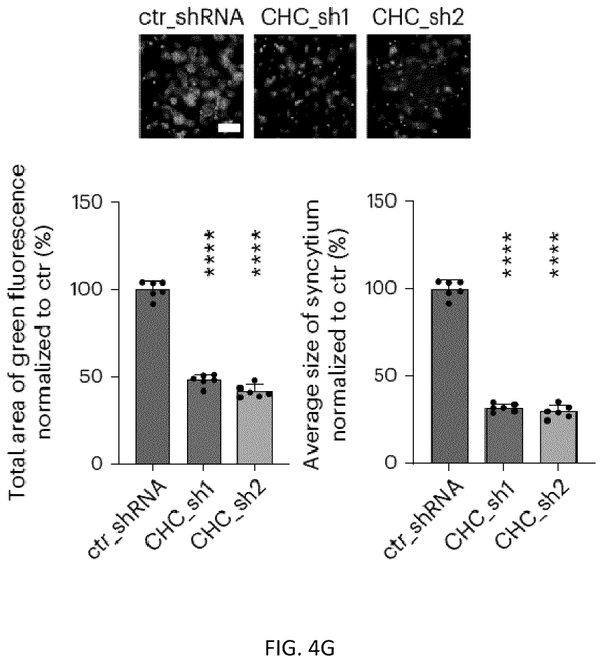

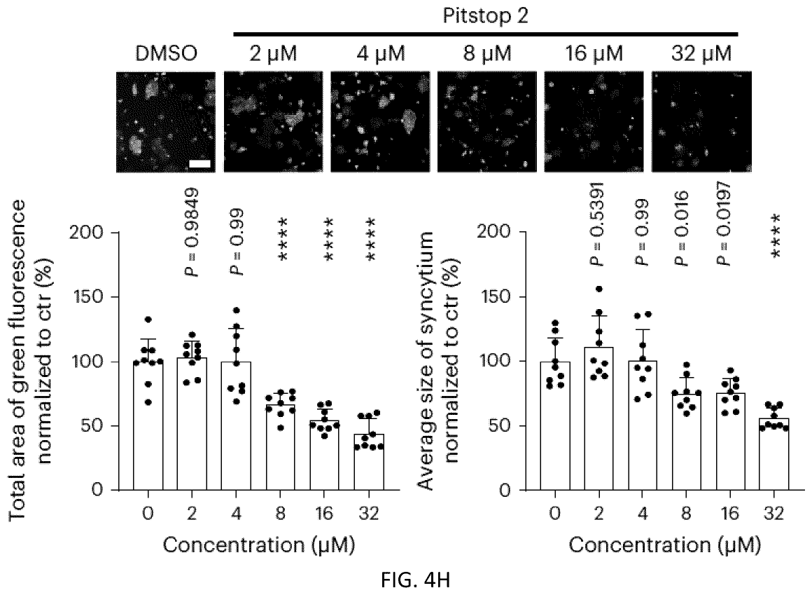

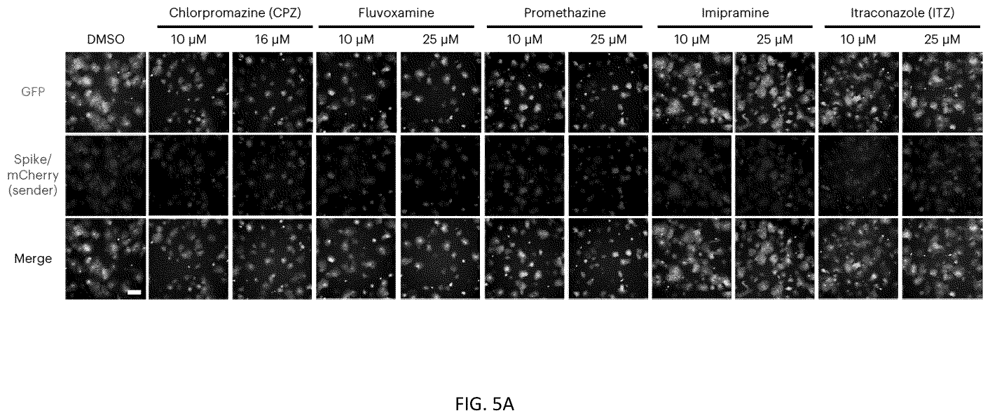

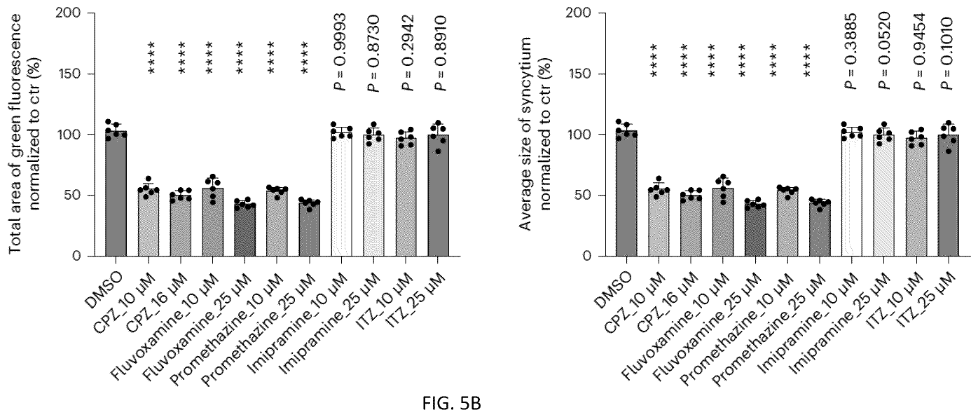

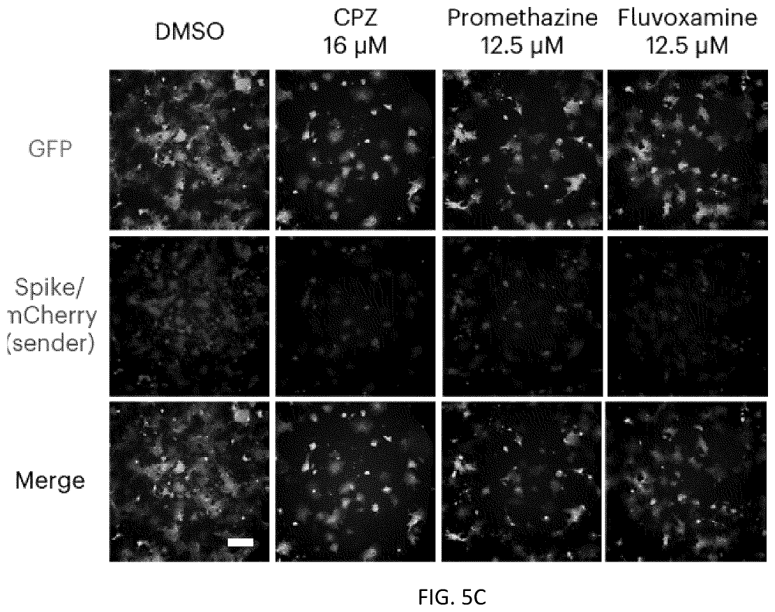

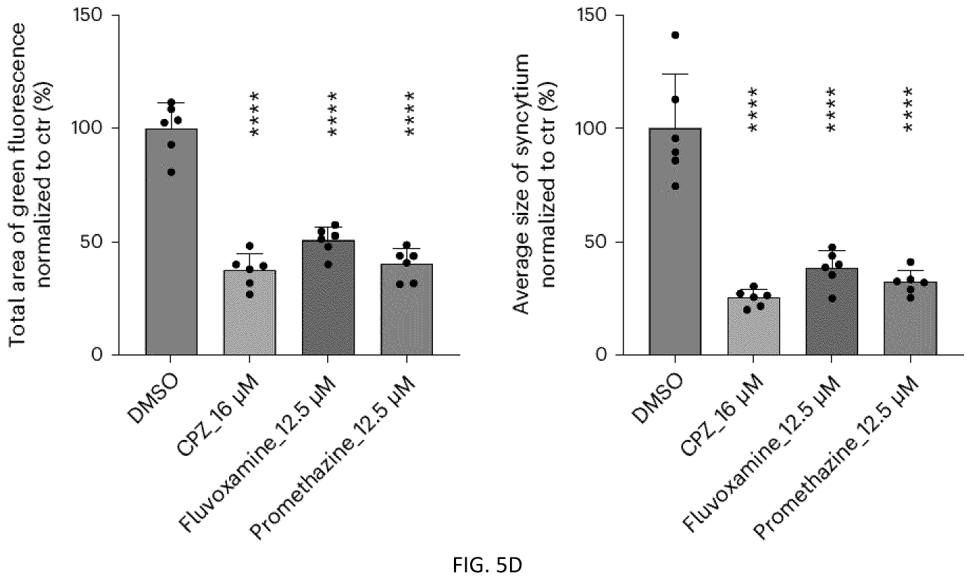

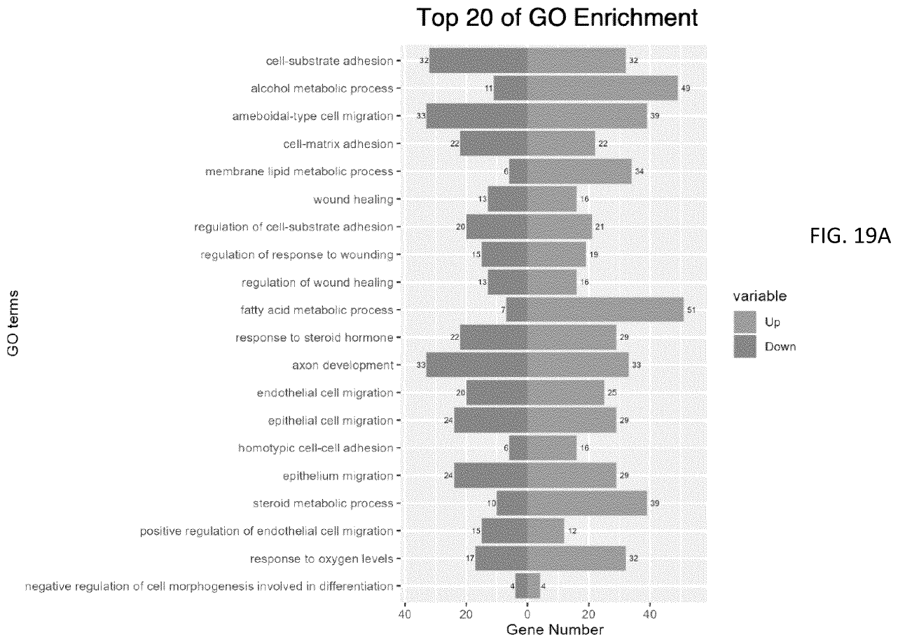

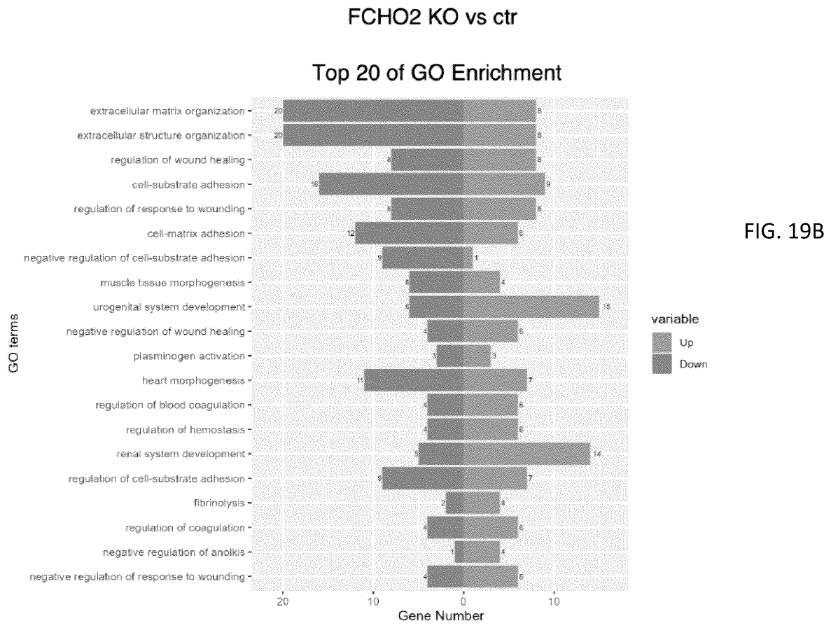

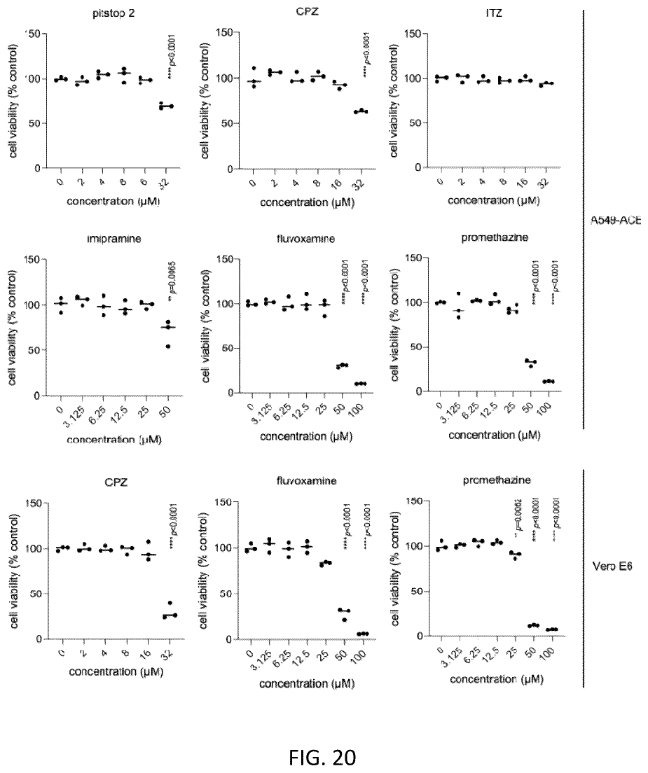

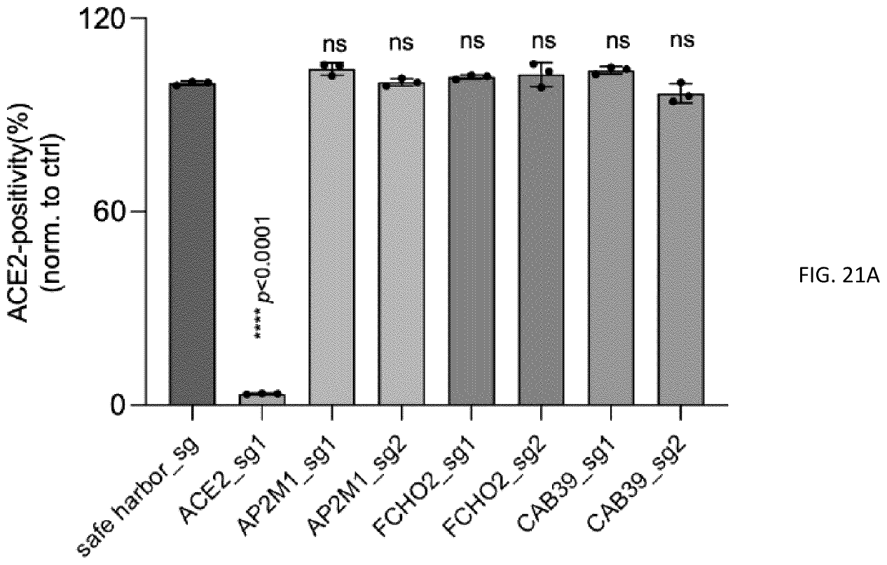

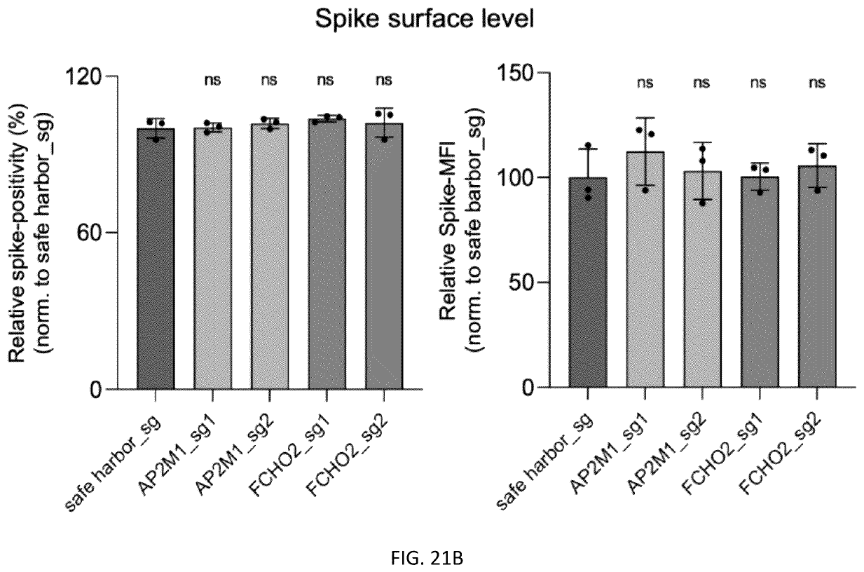

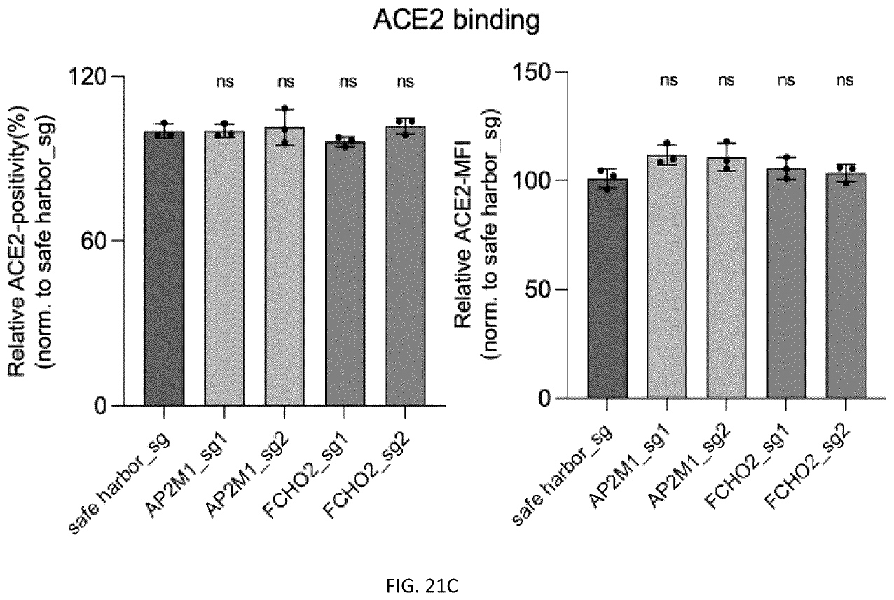

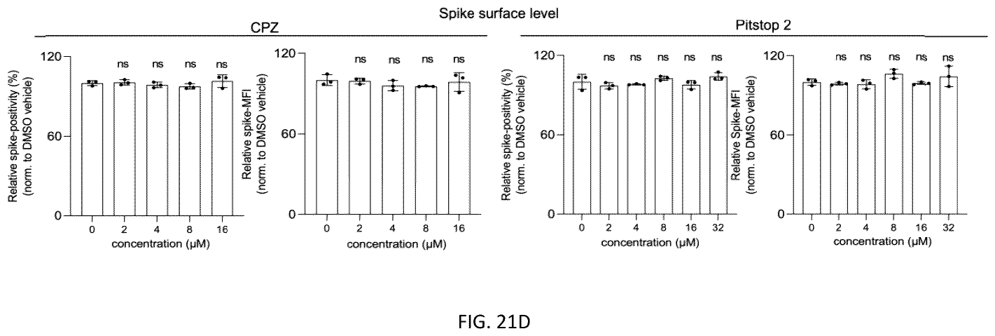

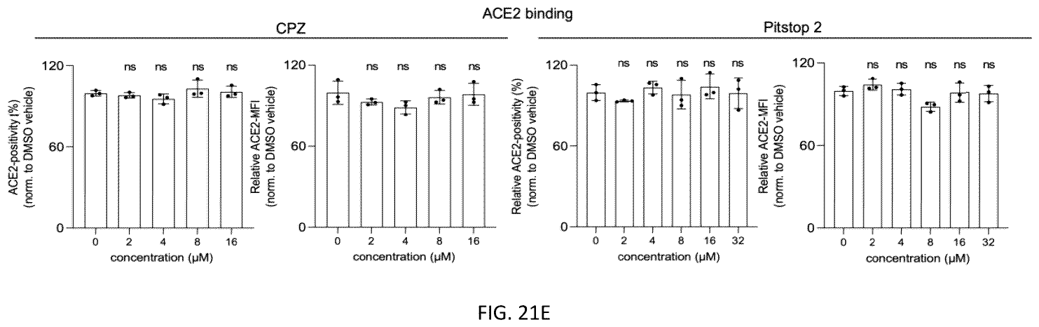

AP2M1 and FCHO2 are core regulators of clathrin-mediated endocytosis (CME)+8.49. RNA-seq and gene ontology (GO) enrichment analysis on FCHO2 and AP2M1 knockout A549-ACE2 cells revealed the positive regulation of cell-substrate/matrix adhesion, among other processes, in both FCHO2 and AP2M1 knockout cells (FIG. 19). Cell-substrate/matrix adhesion was reported to increase the force required for deforming a membrane during clathrin-coated vesicle formation and inhibit CME50. We further showed that genetic knockdown of clathrin heavy chain (CHC) (FIGS. 4E-G) and treatment with a clathrin inhibitor Pitstop 2 (FIG. 4H) both suppressed the cell-cell fusion process. These results support the involvement of CME in spike-induced syncytium formation. We moved on to test approved drug candidates that could inhibit spike-induced syncytium formation. A previous study showed that the CME inhibitor promethazine reduces SARS-COV-2-induced cytotoxicity in Vero E6 cells²⁵. In a drug screen with more than 3,000 approved drugs, promethazine, fluvoxamine and itraconazole (ITZ) were scored to have some inhibitory effects on SARS-COV-2-induced syncytium formation in Vero E6 cells, albeit not further characterized in the study¹⁹. Here we carried out a more comprehensive evaluation of the effect of five approved endocytosis inhibitors and found that treatment with the three CME inhibitors (that is, chlorpromazine (CPZ), fluvoxamine and promethazine, but not Imipramine and ITZ which both primarily affect micropinocytosis) greatly impeded syncytium formation in both A549-ACE2 and Vero E6 cells (FIG. 5A-D). The drug doses used did not affect cell viability (FIG. 20). The decrease in syncytia observed after CME inhibition did not appear to be due to an effect on the ACE2 surface level in the receiver cells or spike's localization to cell surface for ACE2 binding (FIG. 21A-E). Future work may examine whether it involves actin cytoskeletal rearrangement as previously described for myoblast cell-cell fusion⁵¹.

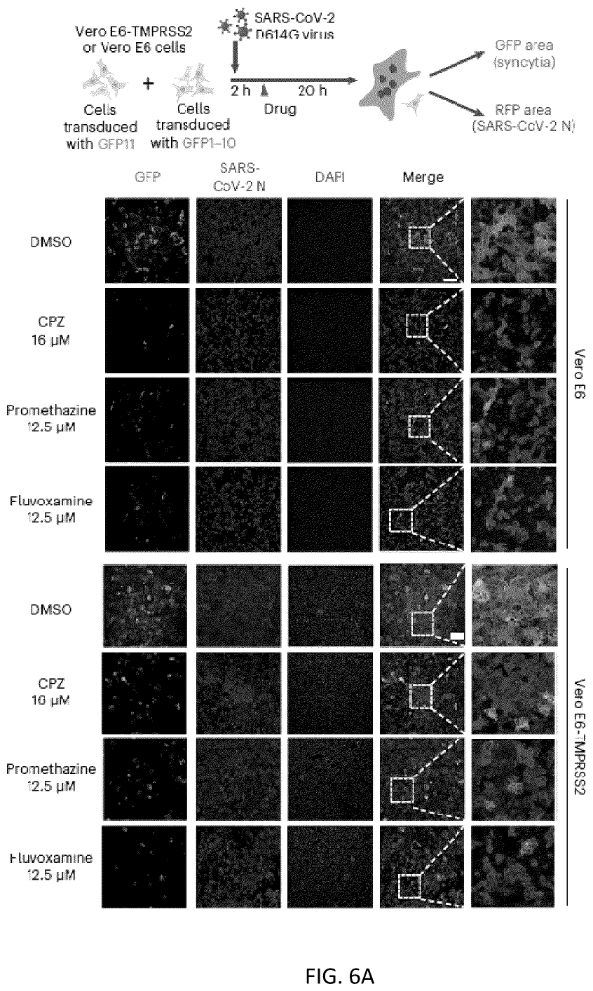

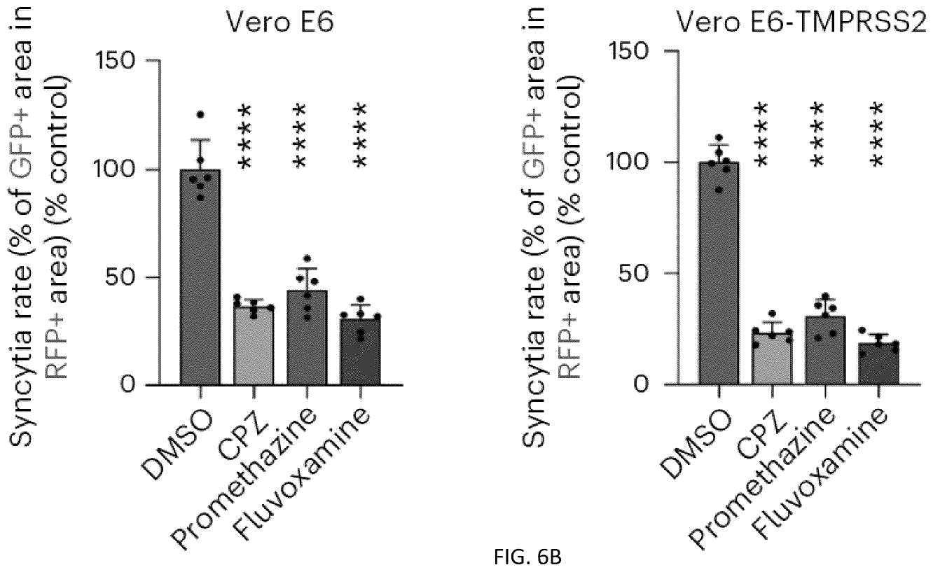

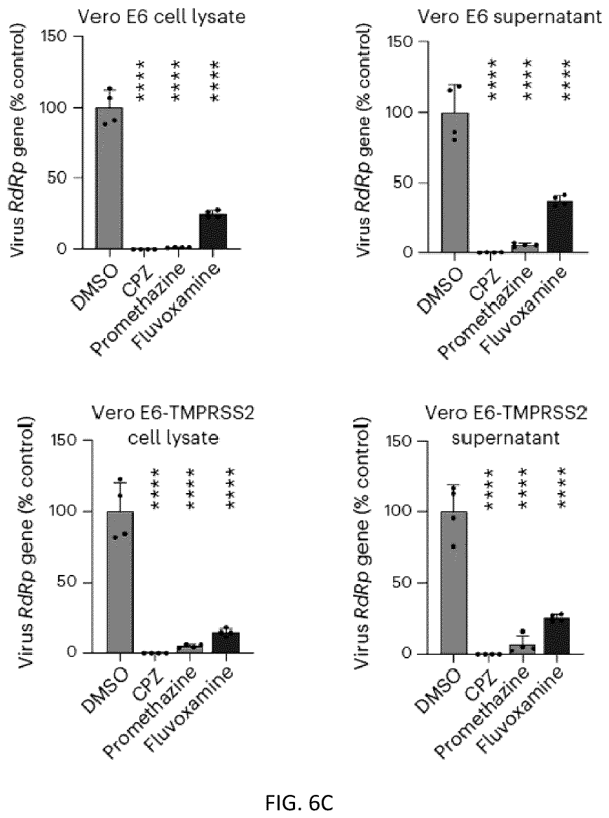

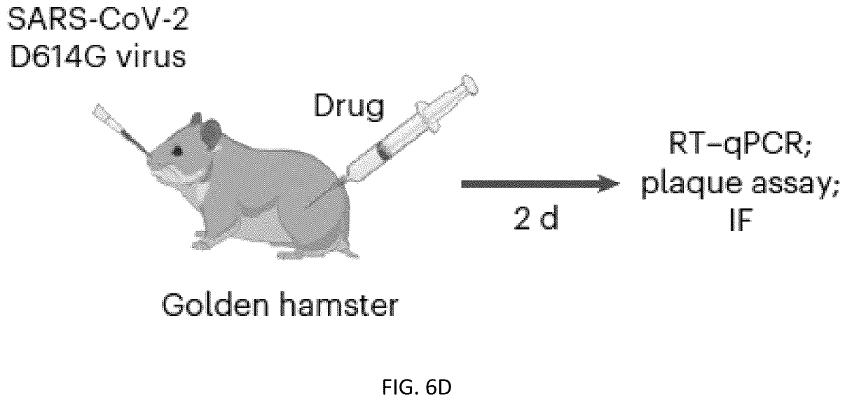

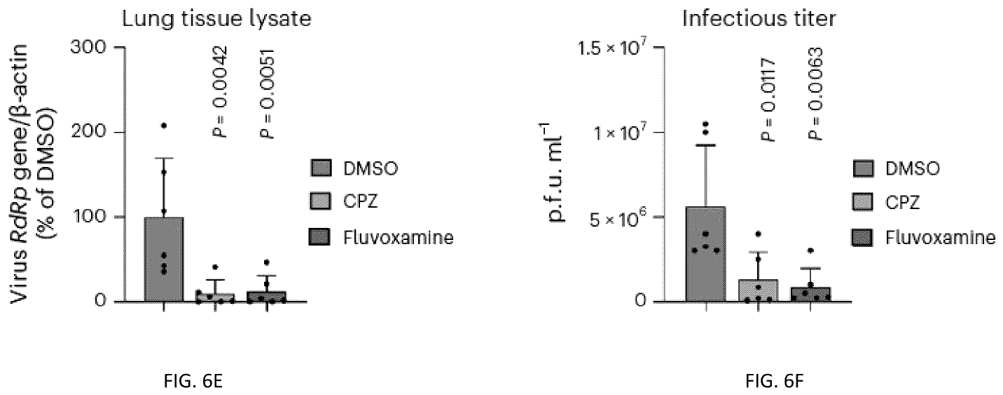

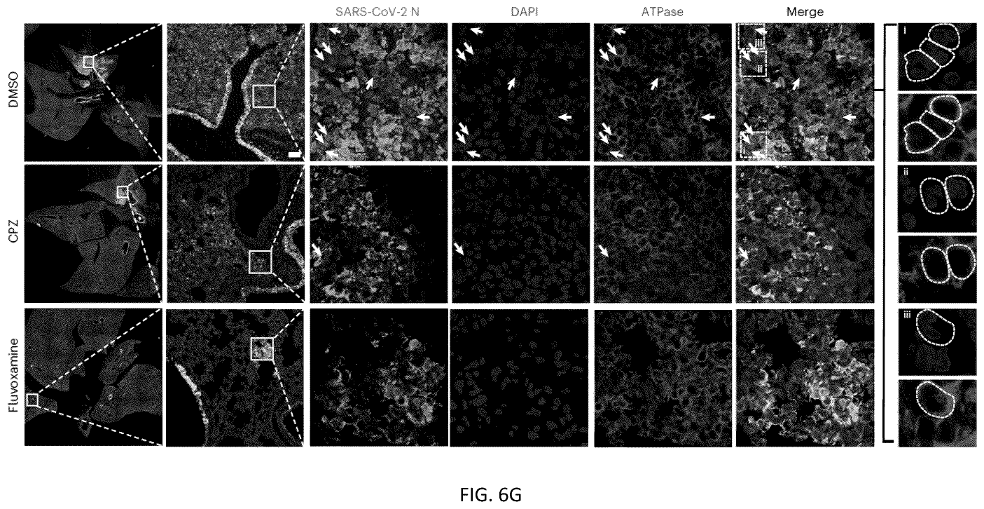

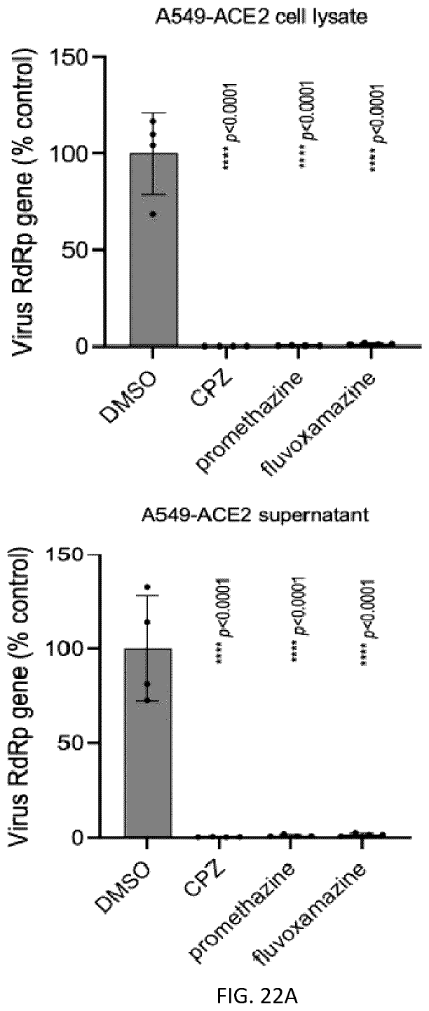

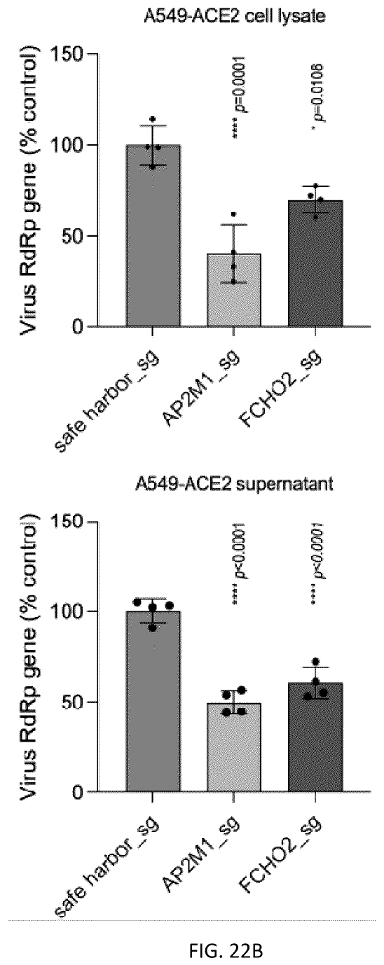

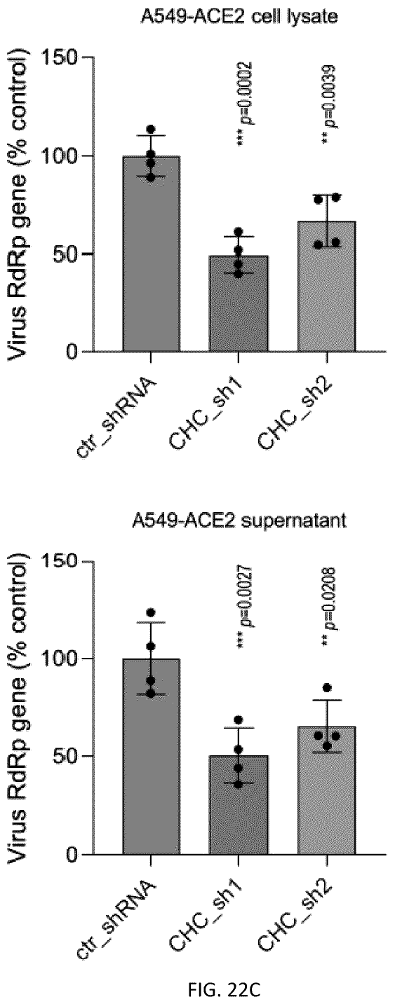

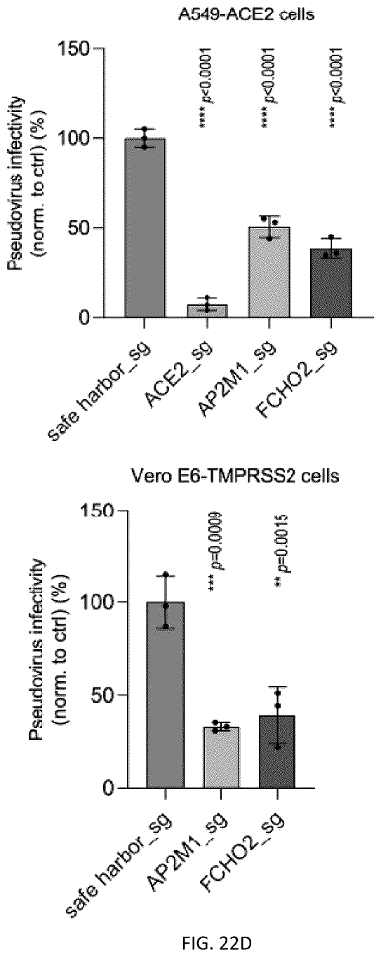

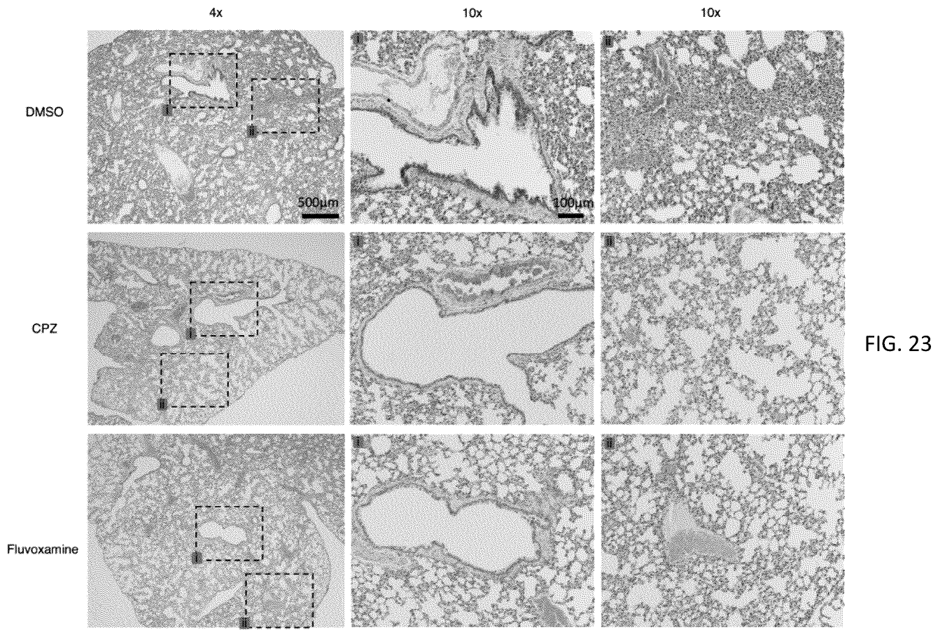

We extended our work to validate our findings using live SARS-COV-2 both in vitro and in vivo. We confirmed that treatment with all three CME inhibitors (CPZ, fluvoxamine and promethazine) inhibited syncytium formation in the SARS-COV-2 D614G-infected cells (FIG. 6An B). In addition, the three inhibitors also greatly reduced the expression levels of SARS-COV-2 RNA-dependent RNA polymerase (RdRp) in both cell lysate and supernatant samples after virus infection (FIG. 6C and FIG. 22A), indicating the reduced production of new viruses. Similarly, FCHO2 and AP2M1 knockout (FIG. 22B), as well as CHC knockdown (FIG. 22C), reduced the RdRp levels in the SARS-COV-2-infected cells. Since FCHO2 and AP2M1 knockout cells were also less susceptible to the spike-pseudotyped virus (FIG. 22D), CME inhibition exerts anti-viral effects probably via reducing viral entry, in addition to reducing syncytium formation. In our in vivo experiments (FIG. 6D), treatment with CPZ and fluvoxamine reduced the virus RdRp gene expression level (FIG. 6E), the infectious SARS-COV-2 titer (FIG. 6F), the area of positivity of the SARS-COV-2 nucleocapsid protein (FIG. 6G) and bronchiolar epithelium damage, alveolar congestion, infiltration and hemorrhage (FIG. 23) in the lung tissues of the virus-infected hamsters. Also, less syncytium-like multinucleated cells were detected within the SARS-COV-2 nucleocapsid-positively stained lung tissues in the CPZ- and fluvoxamine-treated hamsters (FIG. 6G). These results support the idea that the CME inhibitors reduce the viral load, spread, syncytium formation and pathogenesis in the SARS-COV-2-infected tissues, and demonstrate the in vivo relevance of our findings.

Collectively, our results underscore the involvement of the CME machinery in driving the cell-cell fusion process. These results also validate our reciprocal high-throughput screening approach for identifying host factors in receiver cells that are pivotal for syncytium formation. Using our method could also uncover gene knockouts that may enhance syncytium formation (FIG. 22A-C), although this is not the focus of the current study. CRISPR activation screening in receiver cells, as well as CRISPR knockout and activation screening in sender cells, could be performed to identify additional determinants.

### 6.5 Discussion

Here we have established droplet microfluidics and size-exclusion selection strategies to enable two-way reciprocal screening of variant libraries in a sender-receiver cell fusion system. Our pool-based methods are scalable for quantitative assessment of cell-cell fusion, bypassing the laborious steps to individually construct and characterize genetic mutants and perturbations. Functional annotation of mutations that confer SARS-COV-2 spike with greater syncytium-forming potential should aid evaluation of the pathological consequences of the existing and emerging viral variants. So far, previous work primarily focused on studying how mutations at the RBD region and the ectodomain of the spike protein affect ACE2 binding affinity and/or antibody escape using phage or yeast surface display⁵²⁻⁵⁸. Extending the efforts to function-ally annotate the other parts of the spike protein that are not in direct contact with the ACE2 receptor and more importantly under human cell environments, we scanned the FPPR and furin cleavage site region of the spike protein and revealed the presence of syncytium-enhancing mutations at both regions. Our work indicates that single mutations at the non-RBD region including FPPR and furin cleavage-site region of the spike is sufficient in enhancing its syncytium-forming ability. Of important note, the K854H substitution indeed confers Omicron's spike with syncytium-forming potential comparable to that of the D614G strain, while sites including 683, 684, 687, 688 and 854 were predicted to have mutability scores comparable or close to that of 681 at which mutations were found in Omicron and Delta variants, and other mutations at the RBD of spike. Emergence of syncytium-promoting mutations at these sites in any future viral isolates should be scrutinized. Although DMS over the entire spike sequence is challenging given its large size to generate all the possible variants (that is, 20 amino acid residues×1,273 positions), future work could couple our scalable screening systems established for studying syncytia in human cells with deep mutation learning⁵⁹ and high-order combinatorial mutagenesis⁶⁰ methods to functionally annotate mutations at the other regions of the spike that have high mutability as well as combinatorial mutational effects to map epistatic relationships.

To identify the cellular determinants of SARS-COV-2 spike-induced syncytium formation, we performed a whole-genome CRISPR screening and identified two core CME regulators, FCHO2 and AP2M1, as key factors, in addition to the ACE2 receptor. Inhibition of syncytium formation upon treatment with a selective CME inhibitor Pitstop 2 further affirms the involvement of CME in this process. The CRISPR screen performed in this study was designed to specifically look for determining factors for spike-induced syncytium formation given its impact on disease severity, which differs from previous CRISPR screens²²⁻²⁸ that gave an overview of host factors involved in the virus infection process and life cycle; thus, more and different hits may be identified in those screens. While we found that ACE2 and a few other CME-related genes (APIG1, APIBI, AAGAB) were scored as hits in the previous SARS-COV-2 virus infection-based CRISPR screens²⁶⁻²⁸, the potent syncytium formation-modifying hits (including FCHO2 and AP2M1) identified in our screen were not previously uncovered, emphasizing that different aspects of viral biology are revealed by these screening methods. With the intention to case COVID-19 severity via combating syncytium formation, pharmacological treatment using CME inhibitors could be an important option to consider. CPZ and fluvoxamine are widely used drugs for treating psychiatric disorders, and are also known to disrupt CME⁶¹. Our results showed that CME inhibitor treatment is effective in suppressing syncytium formation induced by spike. Taken together, our genetic data demonstrating the involvement of the CME machinery in driving SARS-CoV-2 spike-induced syncytium formation provide a plausible drug mechanism-of-action to support the repurposing of CPZ⁶², fluvoxamine⁶³ and other potential CME inhibitors for alleviating COVID-19 severity in patients.

More broadly, our paired-cell profiling systems can be applied to study a variety of pathological, physiological and even synthetic conditions that are relevant to biomedical applications. Apart from SARS-COV-2, a broad spectrum of viruses including human immuno-deficiency virus⁶⁴, Herpesviridae65, respiratory syncytial virus⁶⁶, as well as other Coronaviridae, induces syncytium formation. Fusion was also reported between tumor and normal somatic cells to form hybrid cells that are more malignant and exhibit increased metastatic behaviour⁶⁷. Defining the common and unique determinants for each type of virus-induced syncytium formation and revealing cellular regulators for tumor-normal somatic cell fusion could help combat the disease pathogeneses. Fusion of specific cell types forms multinucleated cells including syncytiotrophoblasts, myotubes and osteoclasts to aid their physiological functions in controlling maternal-fetal material exchange at the placenta, coordinating muscle contraction and facilitating bone resorption, respectively⁶⁸. Artificial fusion of B cells and myeloma cells produces hybridoma as the workhorse for antibody production⁶⁹. Fusion of human embryonic stem cells with somatic cells reprograms them to pluripotency as cell sources for regenerative medicine⁷⁰. Fusion of dendritic cells with tumor cells produces hybrids that express the tumor-associated antigens and is being tested as potential cancer immunotherapy reagents⁶⁷. The application of high-throughput profiling systems together with CRISPR screening will aid the understanding of the mechanisms by which these different cells fuse and their engineering to achieve greater fusion efficiencies for real-life applications.

### 7. METHODS

### 7.1 Plasmid Construction

All the constructs used in this study were generated with standard cloning strategies, including PCR, overlapping PCR, oligo annealing, digestion and ligation. Primers were purchased from Genewiz. The plasmid sequence was verified by Sanger sequencing. The pCAG-spike(D614G)-GFP11-mCherry plasmid was modified from Addgene plasmid 158761. Briefly, GFP11 and mCherry sequences were amplified from Addgene plasmid 68716 and 79124, respectively, and then cloned into the downstream of spike (D614G), resulting in the pCAG-spike-GFP11-P2A-mCherry vector. The pCAG-spike(Omicron)-GFP11-mCherry plasmid was modified from pCAG-spike(D614G)-GFP11-mCherry plasmid, that is, the spike (Omicron) cassette was amplified from Addgene plasmid 179907 to replace spike(D614G). For the pCMV-BSD-GFP1-10 plasmid, BSD and GFP1-10 sequences were amplified from Addgene plasmid 68761 and 70224, respectively, and then cloned into the pFUGW lentiviral vector backbone, resulting in the Lenti-pGMV-BSD-p2A-GFP1-10 vector. To generate lenti-viral vectors expressing an sgRNA that targets a specific gene, oligo pairs for the target sequence were annealed and cloned into the BbsI restriction sites in Addgene plasmid 67989. To generate lentiviral vectors express-ing shRNAs, oligo pairs for CHC were annealed and cloned into EcoRI/Agel restriction sites in Addgene plasmid 10879. The plasmids, sgRNA sequences and primers used are listed in Supplementary Tables 1 and 2.

### 7.2 Virus and Biosafety

The SARS-COV-2 D614G virus strain was isolated from laboratory-confirmed COVID-19 patients in Hong Kong. The virus was cultured using Vero E6-TMPRSS2 cells and titred by plaque assays. All experiments with SARS-COV-2 were performed according to the approved standard operating procedures of the Biosafety Level 3 facility at the Department of Microbiology, School of Clinical Medicine, The University of Hong Kong.

### 7.3 Cell Culture and Generation of Cell Line

HEK293T (ATCC) and Vero E6 cells were grown in DMEM medium containing 10% FBS and 1×penicillin-streptomycin. A549-ACE2 cells and Vero E6-TMPRSS2 cells were cultured with DMEM medium (with 10% FBS and 1× penicillin-streptomycin) supplemented with 0.5 μg ml-1 puromycin (ant-pr-1, InvivoGen) or 1 mg ml-1 G418 (ant-gn-1, InvivoGen), respectively. All cells were incubated at 37° ° C. with 5% CO2. A549-ACE2-Cas9-GFP1-10 and Vero E6-TMPRSS2-Cas9-GFP1-10 cells were generated by co-transducing pA Wp30 (Addgene, 73857) and Lenti-GFP1-10-blast (pBW93) into the A549-ACE2 and Vero E6-TMPRSS2 cells, respectively, followed by selection with 500 μg ml-1 zeocin (R25001, Life Technologies) and 10 μg ml-1 blasticidin (ant-bl-05, InvivoGen) for ˜10 d for stable Cas9- and GFP1-10-integrated cells. HEK293T-spike-GFP11-P2A-mCherry cells were generated by trans-ducing Lenti-spike-GFP11-P2A-mCherry into HEK293T cells. Single mCherry-positive colonies were expanded. The monoclonal cell line with high fusogenicity was screened by mixing with A549-ACE2 cells. A549-ACE2-GFP1-10-BFP cells were generated by transducing Lenti-BFP and lenti-GFP1-10-blast into A549-ACE2 cells, followed by selection with 10 μg ml-1 blasticidin for ˜10 d and then screening for cells with homogenous BFP expression by FACS.

### 7.4 Droplet Microfluidic System

The microfluidic device was fabricated using a typical soft lithography replica moulding technique. First, two channel moulds designed for two layers of channels were fabricated on two silicon wafers (N100) with SU-8 photoresist (2025, MicroChem) using maskless lithogra-phy (SF-100 Xcel, Intelligent Micro Patterning). The bottom layer for the multiple droplet generator was fabricated with a height of 60 μm, while the height of the top layer for droplet convergence was 70 μm. Then the PDMS pre-polymer base (Sylgard 184, Dow Corning) was crosslinked with the curing agent using the weight ratio of 10:1. After sufficient mixing by a conditioning mixer (AR-100, THINKY), the mixture was poured onto the channel moulds and cured at 65° C. for 4 h. Subsequently, the two layers of PDMS channels were peeled off from the mould. After inlet and outlet punching, the bottom layer was bonded to a glass substrate (ISOLAB) through oxygen plasma treatment (PDC-002). Then, the top layer was bonded to the bottom layer after alignment under the microscope, followed by heating at 90° C. for 12 h. The whole channel was treated with a hydrophobic agent (Aquapel, PPG) to guarantee stable droplet generation. For sample preparation, cells were trypsinized and resuspended in DMEM medium supple-mented with 10% FBS, 1× penicillin-streptomycin as the inner phase, and 18% OptiPrep (92339-11-2, Merck) was added to prevent cell sedimenta-tion. To encapsulate cells in droplets, a fluorinated oil (HFE 7500, 3M) supplemented with 0.5% (w/w) of surfactant (RAN Biotechnologies) was used as the continuous phase. The flow rates of the cell solution and the oil were precisely controlled at 6,000 μl h-1 and 9,000 μl h-1, respectively, by syringe pumps (neMESYS 290N, CETONI). The droplets with a size of ˜75 μm can thus be generated at a frequency of ˜12 kHz.

### 7.5 DMS Library Construction

To construct the Lenti-pCAG-spike-GFP11-mCherry storage vector, we removed the fragment that encoded residues S673 to K854 on the spike and introduced two Esp3I restriction sites by overlapping PCR, resulting in the Lenti-pCAG-spike(1˜672)-Esp3I-Esp3I-spike(855˜1273)-GFP11-mCherry vector. We designed mutagenic primers containing binding sequences, degenerate NNS codons that tile across FPPR (836˜854) or Furin (673˜691) sites, and Esp3I digestion site flanking the end. Synonymous mutations were introduced to the protein-coding sequence containing Esp3I restriction sites in the construct. Nineteen NNS primers for each site were pooled at an equal molar ratio, resulting in an FPPR-NNS primer pool and a Furin-NNS primer pool. The mutagen-esis FPPR or Furin insert was amplified from Addgene plasmid 158761 by using KARA HiFi HotStart ReadyMix with the FPPR-NNS primer pool/S_DMS-fs1 or S_DMSrs/Furin-NNS primer pool (Supplementary Table 2), respectively, and then cloned into the Esp3I-Esp3I site of the storage vector, resulting in the FPPR and Furin DMS libraries. Overall, the FPPR and the Furin DMS libraries each had 380 variants at the protein level (that is, 608 variants at the nucleotide level).

### 7.6 Spike DMS Library Screening

Around 1.6×106 HEK293T WT cells were transduced with the lentivirus-packaged FPPR or Furin DMS library at an MOI of 0.3 to achieve >500-fold representation for each variant. On day 6 post infection, mCherry-positive cells were sorted out and expanded for further screening. Around 6×105 293T spike DMS cells were collected before mixing. For the droplet microfluidic-based strategy, the inner phase cell solution was pre-mixed A549-ACE2-GFP1-10-BFP cells and HEK293T-spike DMS library cells at concentrations of 6.6×106 ml-1 and 1.6×106 ml-1, respectively. To achieve >500-fold coverage for each spike variant, ˜8 million HEK293T-spike DMS library cells were used for each replicate to ensure that enough droplets contained the paired cells. The generated droplets were collected in a T25 flask and incubated for ˜24 h at 37° C. Furthermore, after droplet breakage, cells were collected and fixed, and GFP⁺ and GFP− cells within the BFP+/RFP+ cell population were then sorted out. For the size-exclusion selection-based strategy, A549-ACE2-GFP1-10-BFP cells and HEK293T-spike DMS library cells were mixed and co-cultured in 12-well plates at concentrations of 1×10⁶ and 5×105 cells per well, respectively. Around 24 or 48 h post cell mixing, the cell mixture was trypsinized and passed through a 70 μm strainer, and large syncytia that remained on the strainer were collected. Then the cells passing through the strainer were subjected to cell sorting, that is, small syncytia (GFP+ cells within the BFP+/RFP+ cell population) were sorted out. To achieve ˜500-fold coverage for each spike variant, ˜6 million HEK293T-spike DMS library cells were used for each replicate.

### 7.7 Genome-Wide CRISPR Screening

The MinLibCas9 library (Addgene, 164896) was used for the CRISPR-mediated gene knockout screen. A549-ACE2-Cas9-GFP1-10 cells (1.5×108) were transduced with lentivirus-packaged Min-LibCas9 sgRNA library at an MOI of 0.3 to achieve ˜500-fold representation for each sgRNA. On day 6 post infection, BFP+ cells were sorted out for further culture. On day 14 post infection, ˜35 million A549-ACE2-Cas9-GFP1-10-sgRNA cells were mixed with HEK293T-spike-GFP11-mCherry cells at a 1:2 ratio. At the same time, ˜15 million A549-ACE2-Cas9-GFP1-10-sgRNA cells were collected before mixing. Then the cell mixture was passaged every 2 d. During each passaging, cells were passed through a 40 μm cell strainer to remove the syncytium clumps to enrich the unfused cells. On day 7 post cell mixing, unfused A549-ACE2-Cas9-GFP1-10-sgRNA cells were collected as the late-timepoint sample.

### 7.8 Sample Preparation for NovaSeq-Based Sequencing

For the fresh cell sample, the genomic DNA was isolated by using Dneasy blood and tissue kits (69504, Qiagen) according to manufacturer instructions. For the fixed cell sample, cells were resuspended in 180 μl buffer ATL and 20 μl proteinase K was added. Then, cells were incubated at 56° ° C. for 1 h and at 90° ° C. for another 1 h, followed by the addition of Rnase A, buffer AL and 96%-100% ethanol. The mixture was passed through a Dneasy Mini spin column (Qiagen), washed and eluted according to manufacturer protocol. A two-step PCR proto-col was used to amplify the sgRNA region or DMS region for Illumina sequencing via a previously published protocol71. Briefly, in the first PCR step, the integrated region containing the sgRNA sequences or the DMS sequences of the FPPR or the Furin site was amplified by using KARA HiFi HotStart ReadyMix. Primers 5′-ACACTCTTTCCCTA

CACGACGCTCTTCCGATCTCTTGTGGAAAGGACGAAACA-3′ (SEQ ID NO: 2) and 5′-GTG ACTGGAGTTCAGACGTGTGCTCTTCCGATCTCTAAAGCGCA

TGCTCCAGAC-3′ (SEQ ID NO: 3) were used for amplifying the sgRNA region. Primers 5′-CACGACGCTCTTCCGATCTCGGACCCCAGTAAACCCTC-3′ (SEQ ID NO: 4) or 5′-CACGACGCTCTTCCGATCTCATGTGAACAATTCATACGAATGTG-3′ (SEQ ID NO: 5) and 5′-CAGACGTGTGCTCTTCCGATCTCCGAATGTCCATCCAGACGTT-3′ (SEQ ID NO: 6) or 5′-CAGACGTGTGCTCTTCCGATCTATTTGTTGGGATGGCAATGGAG-3′ (SEQ ID NO: 7) were used for amplifying the DMS region of the FPPR or the furin cleavage site, respectively. To ensure sufficient coverage, all extracted genomic DNA was used in the first step of PCR, where 800 ng gDNA was added per 50 μl PCR reaction. PCR products were purified by using Agencourt AMoure XP beads (A63881, Beckman Coulter Genomics). Then, the second PCR of 13 cycles was performed to add Illumina adapters and sequencing index to the amplicons using KARA HiFi HotStart ReadyMix. The final PCR products were purified with Agencourt AMoure XP beads. The concentrations of different libraries were quantified by real-time PCR using TB Green Premix Ex Taq (Tli RNascH Plus) (RR420A, Kapa Biosystems) with primers 5′-AATGATACGGCGACCACCGA-3′ (SEQ ID NO: 8) and 5′-CAAGCAGAAGACGGCATACGA-3′ (SEQ ID NO: 9), then the libraries were pooled for Novaseq.

### 7.9 Screen Data Analysis

For the genome-wide CRISPR screen, two previously published meth-ods, MAGeCK45 and JACKS46, were used to rank genes on the basis of RRA scores and gene essentiality score, respectively. sgRNA abundances were assessed in ‘before mixing’ samples vs unfused samples. The top screen hits were determined on the basis of MAGeCK RRA and JACKS scores. Kyoto Encyclopaedia of Genes and Genomes (KEGG) and Gene Ontology (GO) enrichment analyses were performed on genes that exhibited depletion between the plasmid library and the ‘before mixing’ samples. For the DMS screen, the fold change of each variant was calculated by comparing its relative abundance before and after selection according to the DiMsum method72.

### 7.10 DMS Screen Hit Validation

HEK293T WT cells were seeded in 24-well plates at a confluence of ˜70% and transfected with 500 ng plasmid expressing only one DMS variant using FuGene HD transfection reagent (E2312, Promega) according to manufacturer protocol. After 24 h, HEK293T-spike variant cells were mixed with A549-ACE2-GFP1-10-BFP cells at a ratio of 1:10 in 24-well plates. After 24 h, microscopy images of the co-cultured cells were taken using the GE IN Cell Analyzer 6500HS high-throughput imaging system. Cell fusion was quantified by measuring the area of syncytia (GFP+ area) using the GE IN Carta image analysis software (v.2.x) and normalized to the WT variant.

### 7.11 Genome-Wide Knockout Screen Hit Validation

A549-ACE2-Cas9-GFP1-10 cells or Vero E6-TMPRSS2-Cas9-GFP1-10 cells were seeded in 12-well plates at a confluence of ˜30% and transduced with lentivirus expressing sgRNA with 10 μg ml-1 polybrene (TR-1003-G, Sigma). For A549-ACE2 cells, at day 8 post infection, ˜2.5×105 A549-ACE2-Cas9-GFP1-10-sgRNA cells were mixed with 3×104 HEK293T-spike(D614G)-GFP11-mCherry cells or HEK293T-spike(Omicron)-GFP11-mCherry cells in a 24-well plate. After 24 h, microscopy images of the co-culture cells were taken. Cell fusion was quantified by measuring the total area of all the syncytia (GFP+ area) and the average size of syncytia, normalized to the control sample. For Vero E6-TMPRSS2 cells, at day 8 post infection, ˜4×105 HEK293T-spike(D614G)-GFP11-mCherry cells or HEK293T-spike(Omicron)-GFP11-mCherry cells were mixed with 1×104 or 3× 104 Vero E6-TMPRSS2-Cas9-GFP1-10-sgRNA cells in a 24-well plate. After 24 h, the cell mixture was trypsinized and passed through a 70 μm cell strainer for FACS assay. Cell fusion was quantified using GFP+/BFP+ and normalized to the control sample.

### 7.12 Lentivirus Production

For sgRNA library or spike DMS library packaging, HEK293T cells were seeded at ˜80% confluency in a 15-cm dish. Library plasmid (9 μg), 9 μg of pCMV-VSV-G vector and 18 μg of pCMV-dR8.2-dvpr vector were trans-fected using 72 μl polyethylenimine. The medium was changed ˜12 h post transfection. The lentivirus supernatants were collected three times at 48, 72 and 96 h post transfection, then combined and filtered with a 0.45-μm polyethersulfone membrane. The virus was concentrated using an Amicon Ultra-15 centrifugal unit, aliquoted and stored at −80° C. For individual sgRNA or shRNA packaging, HEK293T cells were seeded at ˜80% confluency in 6-well plates. sgRNA plasmid (500 ng), 500 ng of pCMV-VSV-G vector and 1 μg of pCMV-dR8.2-dvpr vector were transfected using 4 μl polyethylenimine. The medium was changed ˜16 h post transfection. Lentivirus supernatants were collected at 48 and 72 h post transfection, then combined and filtered with a 0.45-μm polyethersulfone membrane.

### 7.13 Flow Cytometry and Cell Sorting

BD FACSAria Fusion and BD Influx cell sorters were used for cell sorting. Agilent NovoCyte Advanteon BVYG and ACEA NovoCyte Quanteon analysers were used for analysis. Flowjo (v.10.8.1) was used to analyze data generated from flow cytometry experiments. For cell sorting of samples infected with the sgRNA or spike DMS libraries, cells were trypsinized and resuspended in sorting buffer (PBS with 2% FBS and 2× penicillin-streptomycin) and then collected in collection buffer (DMEM medium with 20% FBS and 2× penicillin-streptomycin). For fixed-cells sorting in spike DMS library, droplets were first broken using 1H,1H,2H,2H-perfluoro-1-octanol (PFO) (370533, Sigma). Drop-lets (2 ml) were aliquoted into 15 ml falcon tubes, with 1 ml of DMEM medium added on top of the oil phase. PFO (600 μl) was added, briefly mixed and centrifuged at 300 g for 30 s. The oil phase at the bottom was removed. After washing twice with PBS, cells were fixed with 4% PFA for 20 min at room temperature. Then cells were washed twice and resuspended in PBS. To prevent cell sedimentation, 10% OptiPrep was added during cell sorting.

### 7.14 Spike and ACE2 Surface Staining

Cells were detached using 0.5 mM EDTA, washed once with 5% FBS in PBS, then blocked with 10% FBS for 1 h at 4° ° C. Next, cells were incu-bated with primary antibody in blocking solution for 1 h at 4° C. Goat anti-ACE2 (1:100) (AF933, R&D) was used to label surface ACE2, and rabbit anti-spike S2 (944-1214 aa) (1:500) (28867-1-AP, Proteintech) was used to label surface spike. After washing twice with PBS, cells were incubated with donkey anti-goat IgG(H+L) cross-adsorbed secondary antibodies conjugated with AF568 (1:1,000) (A-11057, Thermo Fisher) or goat anti-rabbit IgG(H+L) cross-adsorbed secondary antibodies conjugated with AF488 (1:1,000) (A-11008, Thermo Fisher) for 1 h at 4° C. in the dark. After washing twice with PBS, cells were resuspended in PBS for FACS or cell sorting. To evaluate S1 subunit cleavage, mouse anti-spike S1 subunit AF488-conjugated antibody (1:200) (FAB105403G, R&D) was used to label surface S1 subunit for 2 h at 4° C. in the dark. The cells were resuspended in PBS for FACS after being washed with PBS twice. The level of S1 subunit cleavage was calculated using (1−Proportion of cells with S1-positive staining)×100%.

### 7.15 ACE2 Binding Assay

Cells transduced with the FPPR DMS library were detached using 0.5 mM EDTA, washed once with 5% FBS in PBS, then incubated with 100 nM biotinylated human ACE2 protein (10108-H08H-B, SinoBiological) for 2 h at 4° C. After washing twice with PBS, cells were incubated with streptavidin conjugated with AF405 (S32351, Thermo Fisher) for 1 h at 4° C. in the dark. After washing twice with PBS, cells were resus-pended in PBS for FACS or cell sorting.

### 7.16 Cell Viability Assay

A549-ACE2-GFP1-10-BFP and Vero E6 cells were seeded at a concentration of 5,500 cells per well in 100 μl of medium in 96-well plates and incubated overnight. For CPZ (S5749, Selleckchem) and ITZ (S2476, Selleckchem) drug treatments, cells were incubated with various amounts (final concentration: 0 μM, 2 μM, 4 μM, 8 μM, 16 UM and 32 μM) at 37° C. overnight. For promethazine HCl (S4293, Selleckchem) and fluvoxamine (S1336, Selleckchem) drug treatments, cells were incubated with virus amounts (final concentration: 0 μM, 3.125 UM, 6.25 μM, 12.5 μM, 25 μM, 50 μM and 100 μM) at 37° C. overnight. For Pitstop2 (HY-115604, Biosystem), cells were incubated with various amounts (final concentration: 0 μM, 2 μM, 4 μM, 8 μM, 16 μM and 32 μM) at 37° C. for 20 min. Then, cell viability was measured using the 2,3-bis-(2-methoxy-4-nitro-5-sulfophenyl)-2H-tetrazolium-5-carboxanilide (XTT) assay. Briefly, drug-containing medium was replaced with 100 μl of detection solution (100 μl of 1×XTT solution with 0.1% volume ratio of 3 mg ml-1 phenazine methosulfate) and incubated at 37° ° C. for 3 h. Then the absorbance was measured at 470 nm using the Varioskan LUX Multimode microplate reader.

### 7.17 Drug Response Study on Syncytium Formation

For the cell-cell fusion assay, the sender cells used were HEK293T cells transfected with plasmid that expresses SARS-COV-2 spike, GFP11 and mCherry, while the receiver cells used were A549-ACE2-GFP1-10 cells or Vero E6-GFP1-10 cells. HEK293T cells were seeded at a confluence of ˜70% and transfected with D614G spike-GFP11-mCherry vector using FuGene HD transfection reagent according to manufacturer protocol. After 24 h, ˜3×10⁴ HEK293T-spike-GFP11-mCherry cells were mixed with 2.5×105 A549-ACE2-Cas9-GFP1-10 cells or 1.8×105 Vero E6-GFP1-10 cells in 24-well plates. At 3 h after seeding, drugs (CPZ, fluvoxamine, ITZ and promethazine) or dimethyl sulfoxide (DMSO) control were added at the indicated concentrations. After 24 h, images of the cell mixture were taken. Syncytia were quantified by measuring the total area of all the syncytia (GFP⁺ area) and the average size of syncytia, and then normalizing to the control sample. For Pitstop 2, 3×104 A549-ACE2-GFP1-10 cells were incubated with various amounts of Pitstop 2 (0 μM, 2 μM, 4 μM, 8 μM, 16 μM and 32 μM) in FBS-free medium at 37° C. for 20 min. After incubation, drug-containing medium was replaced with fresh medium, and A549-ACE2-GFP1-10 cells were mixed with 4×10⁵ 293T-spike-GFP11-mCherry cells in 24-well plates. After 24 h, images of the cell mixture were taken. Syncytia were quantified by measuring the total area of all the syncytia (GFP+ area) and the average size of syncytia, and then normalizing to the control sample. For cell fusion inhibition assay with authentic SARS-CoV-2, Vero E6-GFP11 plus Vero E6-GFP1-10 cells and Vero E6-TMPRSS2-GFP11 plus Vero E6-TMPRSS2-GFP1-10 cells (6×104 cells per well mixed at a 1:1 ratio) in chamber slide (PEZGS0816; MILLIPORE) were challenged with SARS-COV-2 D614G at an MOI of 0.5 and 0.025, respectively. After 2 h of incubation at 37° C., cells were washed once with PBS and cultured in 1% FBS DMEM with drugs (CPZ 16 μM, promethazine 12.5 μM, fluvoxamine 12.5 μM) for 24 h. Then, the cells were washed once with PBS and fixed with 4% paraformaldehyde for 30 min at room temperature. After fixation, cells were permeabilized in 0.1% Triton X-100 and stained with in-house anti-SARS-COV-2 nucleocapsid (N) antibody (1:3,000) and DAPI. Syncytia rate was quantified using GFP+ area (that is, cell fusion area)/RFP+area (that is, virus-infected area).

### 7.18 Pseudovirus Production and Infectivity Test

HEK293T cells were seeded at ˜80% confluency in a 15-cm dish. Lentiviral backbone plasmid (9 μg) encoding EF1α-EGFP (Addgene, 138152), 15 μg Omicron spike expression plasmid (Addgene, 179907) and 12 μg of pCMV-dR8.2-dvpr vector were transfected using 72 μl polyethylenimine. The medium was changed at ˜12 h post transfection. Lentivirus-containing supernatants were collected at 48, 72 and 96 h post transfection, then combined and passed through a 0.45-μm polyethersulfone membrane. The lentiviral supernatants were concentrated from ˜45 ml to 1 ml using lentivirus precipitation solution (VC100, ALSTEM) according to manufacturer instructions. At day 10 post sgRNA infection (that is, sgRNA-infected cells are BFP+), ˜1×105 A549-ACE2-Cas9-GFP1-10-sgRNA cells or Vero E6-TMPRSS2-Cas9-GFP1-10-sgRNA cells were seeded in 48-well plates and transduced with 80 μl of the concentrated pseudovirus. At day 5 post infection, infection rate was quantified by FACS. The infectivity was quantified using GFP+/BFP+ and normalized to cells transduced with safe harbour-targeting sgRNA.

### 7.19 Western Blot

For western blot analysis, cells were collected and lysed with RIPA lysis buffer containing 1× protease inhibitor cocktail. Equal amounts of extracted protein were separated by 10% SDS-PAGE gel and then transferred onto a polyvinylidene fluoride membrane. CHC and GAPDH were probed using rabbit anti-clathrin heavy chain antibody (ab21679, abcam) (1:900) and rabbit anti-GAPDH antibody (2118S, Cell Signaling) (1:5,000), respectively. Anti-rabbit IgG, HRP-linked secondary antibodies (1:10,000) (7074, Cell Signaling) were used, and enhanced chemiluminescence reagents were used for imaging (1705062, Bio Rad).

### 7.20 RNA Extraction and RT-qPCR

Total messenger RNA from cell lysate, supernatant and hamster lung tissue samples were extracted using the RNeasy mini kit (74106, QIA-GEN) according to manufacturer protocol. QuantiNova Probe RT-PCR kit (208354, QIAGEN) was used to quantify the expression of RdRp. The QuantiNova SYBR Green RT-PCR kit (208154, QIAGEN) was used to quantify the expression of β-actin and GAPDH, which were used as internal controls for normalization. All primer sequences used are listed in Supplementary Table 2.

### 7.21 RNA-seq

RNA-seq was performed at the Centre for PanorOmic Science in the LKS Faculty of Medicine, HKU. Briefly, A549-ACE2-Cas9 cells were infected with AP2M1_sgRNA, FCHO2_sgRNA or safe harbor_sgRNA. On day 9 post infection, total mRNA was extracted using TaKaRa MiniBEST Universal RNA extraction kit (9767, TaKaRa) following manufacturer protocol. The complementary DNA library was prepared using KAPA mRNA HyperPrep kit and sequenced on an Illumina NovaSeq 6000 system. Three replicates were sampled for each group. Transcripts abundance was quantified using Kallisto73. Then, gene abundance was quantified using tximport74. DESeq2 was used to identify differentially expressed genes (DEGs)75. Genes with fold change >1.2 or <0.8 and Padj <0.05 were defined as DEGs (Supplementary Data). GO enrichment analysis was performed on DEGs identified from either AP2M1 KO or FCHO2 KO samples using the R (v.2021.09.2+382) package clusterProfiler (v.4.4.4).

### 7.22 Immunofluorescence and Histology Staining

For cultured cells, the SARS-COV-2-infected cells were fixed in 10% formalin for 30 min. After fixation, cells were washed twice with PBS and permeabilized with 0.1% Triton X-100 at 4° C. for 10 min. Then, cells were washed twice with PBS, followed by blocking with 2% BSA at room temperature for 1 h. Next, the cells were incubated with in-house rabbit anti-SARS-COV-2 N antibody (1:3,000) overnight at 4° C. After washing with PBS three times, cells were incubated with goat anti-rabbit secondary antibody (1:1,000; A-11008, Thermo Fisher) for 1 h at room temperature. DAPI was used for nuclear staining. Images were acquired using a Nikon Ti2-E widefield microscope. For hamster lung tissues, the SARS-CoV-2-infected hamster lungs were collected and fixed in 10% formalin for 24 h. Immunofluorescence staining was performed following a previously published method⁷⁶. The in-house guinea pig anti-SARS-COV-2 N antibody (1:1,000) was used to identify SARS-CoV-2 and the rabbit anti-sodium potassium ATPase antibody (1:500; ab76020, Abcam) was used to detect cell membrane. DAPI was used to stain nuclei. The following secondary antibodies were used: goat anti-guinea pig-AF488 (1:1,000; A-11073, Thermo Fisher) and goat anti-rabbit-AF568 (1:1,000; A-11011, Thermo Fisher). Images were acquired using an LSM900 inverted confocal microscope. For hematoxylin and cosin staining, hamster lung section samples were dewaxed and stained with Gill's hematoxylin and eosin-Y following a previously published method76. Images were acquired using an Olympus BX53 light microscope.

### 7.23 Golden Hamster Model

The animal study was approved by the Committee on the Use of Live Animals in Teaching and Research of The University of Hong Kong, and the experiments conducted complied with all relevant ethical regulations. Golden Syrian hamsters (aged 4-6 weeks, male) were obtained from the Chinese University of Hong Kong Laboratory Animal Service Centre through the HKU Centre for Comparative Medicine Research. The drugs were dissolved in DMSO and diluted in PBS. Hamsters were pretreated with one dose of chlorpromazine (10 mg kg-1), fluvoxamine (20 mg kg-1) or DMSO intraperitoneally 24 h before virus inoculation. Then, the hamsters were intranasally challenged with 5×103 plaque-forming units (p.f.u.) SARS-COV-2 D614G prediluted in 50 μl PBS under ketamine (100 mg kg-1) and xylazine (10 mg kg-1) anaesthesia. The infected hamsters were treated with chlorpromazine, fluvoxamine or DMSO at 3 h and 24 h post infection. The body weight of the hamsters was monitored daily and no significant change was observed. Hamsters were killed on day 2 after infection. Lung tissues from infected hamsters were collected for subsequent immunofluorescence staining, RT-qPCR analysis or plaque assay as previously described⁷⁶.

### 7.24 Plaque Assay

The plaque assays were conducted as previously described⁷⁶. In brief, infected hamster lung tissues were homogenized in DMEM using Tissue Lyzer II and supernatants were collected and serially diluted 10-fold. Vero E6-TMPRSS2 cells were seeded in 12-well plates at day −1. After overnight culture, cells were inoculated with the diluted supernatants for 2 h at 37° C., washed with PBS three times and covered with 2% agarose/PBS mixed with DMEM/2% FBS at a 1:1 ratio. The cells were incubated at 37° C. for 48 h and fixed with 4% paraformaldehyde. The fixed samples were stained with 0.5% crystal violet in 25% ethanol/distilled water to visualize plaque formation.

### 7.25 Mutability Prediction

The mutability scores of the direct coupling analysis (DCA) and independent (IND) models of the FPPR and Furin cleavage sites were calculated using the code constructed in ref. 77 (https://github.com/juan-rodriguez-rivas/covmut). Covmut_proteome.py was run using Uniref100 as the ‘distant’ database and a representative version of the GISAID spike variant amino acid sequence database updated to 20 Sep. 2022 as the ‘close’ database. To construct the ‘close’ database, CD-HIT78 was used to cluster the sequences of GISAID spike variants at 90% identity and generated 34,182 representative sequences. The resultant mutability scores were compared with the fold change (FC) values obtained from our spike DMS library profiling. The mutability scores of the DCA and IND models of RBD were collected from https://github.com/GiancarloCroce/DCA_SARS-COV-2 (ref. 77)

### 7.26 Molecular Modelling

The SARS-COV-2 spike single amino acid substitutions on the FPPR were introduced using the mutagenesis wizard programme of PyMol. PyMol was used for atom-atom distance measurement and visualization of the protein model.

### 7.27 Statistical Analysis

Statistical analyses were performed using GraphPad Prism 9 software. All data are shown as mean±s.d. Statistical significance of differences between more than two groups was calculated using one-way analysis of variance (ANOVA). The number of biological replicates are listed in the figure legends.

### 7.28 Reporting Summary

Further information on research design is available in the Nature Portfolio Reporting Summary in Chan, C. W. F., Wang, B., Nan, L. et al. High-throughput screening of genetic and cellular drivers of syncytium formation induced by the spike protein of SARS-COV-2. Nat. Biomed. Eng (2023).

### 7.29 Data Availability

The main data supporting the results in this study are available within the paper and its Supplementary Information. The molecular structures of the spike proteins of the SARS-CoV-2 variants are available from the Protein Data Bank, with accession codes 6XR8, 7KRQ and 7TO4. The raw and analysed datasets generated during the study are available for research purposes from the corresponding authors on reasonable request. Source data for the figures are provided in Chan, C. W. F., Wang, B., Nan, L. et al. High-throughput screening of genetic and cellular drivers of syncytium formation induced by the spike protein of SARS-CoV-2. Nat. Biomed. Eng (2023). https://doi.org/10.1038/s41551-023-01140-z

SUPPLEMENTARY TABLE 1
Constructs used in this study.

| Constructs used in this study | | |
|---|---|---|
| construct | | |
| ID | Design | Reference |
| CHp178 | pFUGW•CAGp•Spike(D614G)•GFP11•P2A•mCherry | this study |
| CHp229 | pFUGW•CAGp•Spike(omicron)•GFP11•P2A•mCherry | this study |
| pBW93 | pFUGW•CMVp•BSD•P2A•GFP1•10 | this study |
| S_Q836V | pFUGW•CAGp•Spike(D614G, Q836V)•GFP11•P2A•mCherry | this study |
| S_Y837D | pFUGW•CAGp•Spike(D614G, Y837D)•GFP11•P2A•mCherry | this study |
| S_Y837F | pFUGW•CAGp•Spike(D614G, Y837F)•GFP11•P2A•mCherry | this study |
| S_Y837K | pFUGW•CAGp•Spike(D614G, Y837K)•GFP11•P2A•mCherry | this study |
| S_G838K | pFUGW•CAGp•Spike(D614G, G838K)•GFP11•P2A•mCherry | this study |
| S_G838R | pFUGW•CAGp•Spike(D614G, G838R)•GFP11•P2A•mCherry | this study |
| S_G838P | pFUGW•CAGp•Spike(D614G, G838P)•GFP11•P2A•mCherry | this study |
| S_D839Q | pFUGW•CAGp•Spike(D614G, D839Q)•GFP11•P2A•mCherry | this study |
| S_C840G | pFUGW•CAGp•Spike(D614G, C840G)•GFP11•P2A•mCherry | this study |
| S_L841H | pFUGW•CAGp•Spike(D614G, L841H)•GFP11•P2A•mCherry | this study |
| S_L841W | pFUGW•CAGp•Spike(D614G, L841W)•GFP11•P2A•mCherry | this study |
| S_D843G | pFUGW•CAGp•Spike(D614G, D843G)•GFP11•P2A•mCherry | this study |
| S_D843M | pFUGW•CAGp•Spike(D614G, D843M)•GFP11•P2A•mCherry | this study |
| S_D843Y | pFUGW•CAGp•Spike(D614G, D843Y)•GFP11•P2A•mCherry | this study |
| S_I844D | pFUGW•CAGp•Spike(D614G, I844D)•GFP11•P2A•mCherry | this study |
| S_I844M | pFUGW•CAGp•Spike(D614G, I844M)•GFP11•P2A•mCherry | this study |
| S_A845N | pFUGW•CAGp•Spike(D614G, A845N)•GFP11•P2A•mCherry | this study |
| S_A845T | pFUGW•CAGp•Spike(D614G, A845T)•GFP11•P2A•mCherry | this study |
| S_D843G | pFUGW•CAGp•Spike(D614G, D843G)•GFP11•P2A•mCherry | this study |
| S_D848H | pFUGW•CAGp•Spike(D614G, D848H)•GFP11•P2A•mCherry | this study |
| S_D848R | pFUGW•CAGp•Spike(D614G, D848R)•GFP11•P2A•mCherry | this study |
| S_D848T | pFUGW•CAGp•Spike(D614G, D848T)•GFP11•P2A•mCherry | this study |
| S_L849N | pFUGW•CAGp•Spike(D614G, L849N)•GFP11•P2A•mCherry | this study |
| S_I850H | pFUGW•CAGp•Spike(D614G, I850H)•GFP11•P2A•mCherry | this study |
| S_I850M | pFUGW•CAGp•Spike(D614G, I850M)•GFP11•P2A•mCherry | this study |
| S_A852M | pFUGW•CAGp•Spike(D614G, A852M)•GFP11•P2A•mCherry | this study |
| S_Q853R | pFUGW•CAGp•Spike(D614G, Q853R)•GFP11•P2A•mCherry | this study |
| S_Q853V | pFUGW•CAGp•Spike(D614G, Q853V)•GFP11•P2A•mCherry | this study |
| S_K854C | pFUGW•CAGp•Spike(D614G, K854C)•GFP11•P2A•mCherry | this study |
| S_K854L | pFUGW•CAGp•Spike(D614G, K854L)•GFP11•P2A•mCherry | this study |
| S_K854N | pFUGW•CAGp•Spike(D614G, K854N)•GFP11•P2A•mCherry | this study |
| S_K854Q | pFUGW•CAGp•Spike(D614G, K854Q)•GFP11•P2A•mCherry | this study |
| S_K854R | pFUGW•CAGp•Spike(D614G, K854R)•GFP11•P2A•mCherry | this study |
| S_K854T | pFUGW•CAGp•Spike(D614G, K854T)•GFP11•P2A•mCherry | this study |
| S_K854W | pFUGW•CAGp•Spike(D614G, K854W)•GFP11•P2A•mCherry | this study |
| S_K854H | pFUGW•CAGp•Spike(D614G, K854H)•GFP11•P2A•mCherry | this study |
| S_A853W | pFUGW•CAGp•Spike(D614G, A853W)•GFP11•P2A•mCherry | this study |
| S_C840S | pFUGW•CAGp•Spike(D614G, C840S)•GFP11•P2A•mCherry | this study |
| S_D848N | pFUGW•CAGp•Spike(D614G, D848N)•GFP11•P2A•mCherry | this study |
| S_C851S | pFUGW•CAGp•Spike(D614G, C851S)•GFP11•P2A•mCherry | this study |
| S_P681Y | pFUGW•CAGp•Spike(D614G, P681Y)•GFP11•P2A•mCherry | this study |
| S_R683H | pFUGW•CAGp•Spike(D614G, R683H)•GFP11•P2A•mCherry | this study |
| S_A684H | pFUGW•CAGp•Spike(D614G, A684H)•GFP11•P2A•mCherry | this study |
| S_R685F | pFUGW•CAGp•Spike(D614G, R685F)•GFP11•P2A•mCherry | this study |
| S_V687I | pFUGW•CAGp•Spike(D614G, V687I)•GFP11•P2A•mCherry | this study |
| S_A688T | pFUGW•CAGp•Spike(D614G, A688T)•GFP11•P2A•mCherry | this study |
| S_K1255F | pFUGW•CAGp•Spike(D614G, K1255F)•GFP11•P2A•mCherry | this study |
| O_K854H | pFUGW•CAGp•Spike(omicron, K854H)•GFP11•P2A•mCherry | this study |
| O_A846W | pFUGW•CAGp•Spike(omicron, A846W)•GFP11•P2A•mCherry | this study |
| O_C840S | pFUGW•CAGp•Spike(omicron, C840S)•GFP11•P2A•mCherry | this study |
| O_D848N | pFUGW•CAGp•Spike(omicron, D848N)•GFP11•P2A•mCherry | this study |
| O_C851S | pFUGW•CAGp•Spike(omicron, C851S)•GFP11•P2A•mCherry | this study |
| hFurin | pCMV3•huFurin | Sino biological (Cat No. HG10141•ACR) |
| hTMPRSS2 | pCMV3•huTMPRSS2 | Sino biological (Cat No. HG13070•Ch) |
| AWp30 | pFUGW•EFSp•Cas9•P2A•Zeo | Wong, A. S., et al., PNAS. 2016; 113(9): 2544•9 |
| pBW69d | pFUGW•hU6•ACE2_sg1•PGKp•puro•T2A•BFP | this study |
| pBW78d | pFUGW•hU6•safe harbor_sg1•PGKp•puro•T2A•BFP | this study |
| pBW111 | pFUGW•hU6•ACE2_sg3•PGKp•puro•T2A•BFP | this study |
| pBW112 | pFUGW•hU6•CAB39_sg1•PGKp•puro•T2A•BFP | this study |
| pBW113 | pFUGW•hU6•CAB39_sg2•PGKp•puro•T2A•BFP | this study |
| pBW114 | pFUGW•hU6•AP2M1_sg1•PGKp•puro•T2A•BFP | this study |
| pBW115 | pFUGW•hU6•AP2M1_sg2•PGKp•puro•T2A•BFP | this study |
| pBW116 | pFUGW•hU6•FCHO2_sg1•PGKp•puro•T2A•BFP | this study |
| pBW117 | pFUGW•hU6•FCHO2_sg2•PGKp•puro•T2A•BFP | this study |
| pBW128 | pFUGW•hU6•GBP6_sg1•PGKp•puro•T2A•BFP | this study |
| pBW129 | pFUGW•hU6•GBP6_sg2•PGKp•puro•T2A•BFP | this study |
| pBW130 | pFUGW•hU6•RNF2_sg1•PGKp•puro•T2A•BFP | this study |
| pBW131 | pFUGW•hU6•RNF2_sg2•PGKp•puro•T2A•BFP | this study |
| pBW134 | pFUGW•hU6•ZEB1_sg1•PGKp•puro•T2A•BFP | this study |
| pBW135 | pFUGW•hU6•ZEB1_sg2•PGKp•puro•T2A•BFP | this study |
| pBW136 | pFUGW•hU6•UBIAD1_sg1•PGKp•puro•T2A•BFP | this study |
| pBW137 | pFUGW•hU6•UBIAD1_sg2•PGKp•puro•T2A•BFP | this study |
| pBW138 | pFUGW•hU6•non targeting_sg1•PGKp•puro•T2A•BFP | this study |
| pBW139 | pFUGW•hU6•non targeting_sg2•PGKp•puro•T2A•BFP | this study |
| pBW145 | pFUGW•hU6•ACE2_sg•PGKp•puro•T2A•BFP | this study |
| pBW147 | pFUGW•hU6•FCHO2_sg•PGKp•puro•T2A•BFP | this study |
| pBW168 | pFUGW•hU6•NDUFB10_sg1•PGKp•puro•T2A•BFP | this study |
| pBW169 | pFUGW•hU6•NDUFB10_sg2•PGKp•puro•T2A•BFP | this study |
| addgene | pFUGW•EF1a•d2EGFP | Wessels, H. H., et al., Nat Biotechnol. 2020 |
| 138152 | | Jun: 38(6): 722•727. |
| addgene | pFUGW•CAGp•spike(omicron) | Garcia•Beltran, W. F., et al. Cell. 2022 Jan 6. pii. |
| 179907 | | S0092•8674(21)01496•3 |
| addgene | pLKO.1•TRC contorl | Moffat, J., et al. Cell, 2006. 124(6): p. 1283•98. |
| 10879 | | |
| pBW164 | pFUGW•hU6•CHC_sh1•PGKp•puro | this study |
| pBW165 | pFUGW•hU6•CHC_sh2•PGKp•puro | this study |
| pBW166 | pFUGW•hU6•CHC_sh3•PGKp•puro | this study |
| pBW167 | pFUGW•hU6•non targeting_sh•PGKp•puro | this study |
| addgene | pHR•SFFV•GFP1•10 | Kamiyama, D. et al. Nat Commun 7, 11046 (2016). |
| 80409 | | |
| pBW89 | pFUGW•CMVp•BSD•P2A•GFP11x7 | this study |

Supplementary Table 2 discloses SEQ ID NOS 2, 3, 10-41, 8, 9, 42-81, 5, 7, 4, 6, and 82-115, respectively, in order of appearance.

Supplementary Table 2
List of primers and sgRNAs used

| Primers | | |
|---|---|---|
| Name | Sequence | Description |
| BW275-F | ACACTCTTTCCCTACACGACGCTCTTCCGATCTCTTGTG | 1st step PCR |
| | GAAAGGACGAAACA | primers for |
| BW276-R | GTGACTGGAGTTCAGACGTGTGCTCTTCCGATCTCTAA | amplication of |
| | AGCGCATGCTCCAGAC | the sgRNA |
| | | library |
| Nova_i5_1 | AATGATACGGCGACCACCGAGATCTACACCGATTGCA | 2nd step PCR |
| | ACACTCTTTCCCTACACGACGCTCTTCCGATC | primers for |
| Nova_i5_2 | AATGATACGGCGACCACCGAGATCTACACGCAGAACA | addition of the |
| | ACACTCTTTCCCTACACGACGCTCTTCCGATC | illumina |
| Nova_i5_3 | AATGATACGGCGACCACCGAGATCTACACGCCTTCTTA | adapters and |
| | CACTCTTTCCCTACACGACGCTCTTCCGATC | index sequence |
| Nova_i5_4 | AATGATACGGCGACCACCGAGATCTACACCGGAAGAA | |
| | ACACTCTTTCCCTACACGACGCTCTTCCGATC | |
| Nova_i5_5 | AATGATACGGCGACCACCGAGATCTACACCCAGAGAA | |
| | ACACTCTTTCCCTACACGACGCTCTTCCGATC | |
| Nova_i5_6 | AATGATACGGCGACCACCGAGATCTACACGAGGAGAA | |
| | ACACTCTTTCCCTACACGACGCTCTTCCGATC | |
| Nova_i5_7 | AATGATACGGCGACCACCGAGATCTACACCACCAGAA | |
| | ACACTCTTTCCCTACACGACGCTCTTCCGATC | |
| Nova_i5_8 | AATGATACGGCGACCACCGAGATCTACACACGAGGAA | |
| | ACACTCTTTCCCTACACGACGCTCTTCCGATC | |
| Nova_i5_9 | AATGATACGGCGACCACCGAGATCTACACGGTAGGAA | |
| | ACACTCTTTCCCTACACGACGCTCTTCCGATC | |
| Nova_i5_10 | AATGATACGGCGACCACCGAGATCTACACGAACGGAA | |
| | ACACTCTTTCCCTACACGACGCTCTTCCGATC | |
| Nova_i5_11 | AATGATACGGCGACCACCGAGATCTACACTTGCGGAA | |
| | ACACTCTTTCCCTACACGACGCTCTTCCGATC | |
| Nova_i5_12 | AATGATACGGCGACCACCGAGATCTACACCAGTGGAA | |
| | ACACTCTTTCCCTACACGACGCTCTTCCGATC | |
| Nova_i5_13 | AATGATACGGCGACCACCGAGATCTACACTCCTGGAA | |
| | ACACTCTTTCCCTACACGACGCTCTTCCGATC | |
| Nova_i5_14 | AATGATACGGCGACCACCGAGATCTACACTACGCGAA | |
| | ACACTCTTTCCCTACACGACGCTCTTCCGATC | |
| Nova_i5_15 | AATGATACGGCGACCACCGAGATCTACACAGACCGAA | |
| | ACACTCTTTCCCTACACGACGCTCTTCCGATC | |
| Nova_i5_16 | AATGATACGGCGACCACCGAGATCTACACTGGTCGAA | |
| | ACACTCTTTCCCTACACGACGCTCTTCCGATC | |
| Nova_i7_1 | CAAGCAGAAGACGGCATACGAGATTTGGTGGAGTGAC | |
| | TGGAGTTCAGACGTGTGCTCTTCCGATC | |
| Nova_i7_2 | CAAGCAGAAGACGGCATACGAGATTTGGCACTGTGAC | |
| | TGGAGTTCAGACGTGTGCTCTTCCGATC | |
| Nova_i7_3 | CAAGCAGAAGACGGCATACGAGATTTGGATCCGTGAC | |
| | TGGAGTTCAGACGTGTGCTCTTCCGATC | |
| Nova_i7_4 | CAAGCAGAAGACGGCATACGAGATTTGACGTCGTGAC | |
| | TGGAGTTCAGACGTGTGCTCTTCCGATC | |
| Nova_i7_5 | CAAGCAGAAGACGGCATACGAGATTTGAGCTGGTGAC | |
| | TGGAGTTCAGACGTGTGCTCTTCCGATC | |
| Nova_i7_6 | CAAGCAGAAGACGGCATACGAGATTTGAGGCAGTGAC | |
| | TGGAGTTCAGACGTGTGCTCTTCCGATC | |
| Nova_i7_7 | CAAGCAGAAGACGGCATACGAGATTTATCGGCGTGAC | |
| | TGGAGTTCAGACGTGTGCTCTTCCGATC | |
| Nova_i7_8 | CAAGCAGAAGACGGCATACGAGATTTACGGAGGTGAC | |
| | TGGAGTTCAGACGTGTGCTCTTCCGATC | |
| Nova_i7_9 | CAAGCAGAAGACGGCATACGAGATTTACAGCCGTGAC | |
| | TGGAGTTCAGACGTGTGCTCTTCCGATC | |
| Nova_i7_10 | CAAGCAGAAGACGGCATACGAGATTTAGCCGAGTGAC | |
| | TGGAGTTCAGACGTGTGCTCTTCCGATC | |
| Nova_i7_11 | CAAGCAGAAGACGGCATACGAGATTCTCCAAGGTGAC | |
| | TGGAGTTCAGACGTGTGCTCTTCCGATC | |
| Nova_i7_12 | CAAGCAGAAGACGGCATACGAGATTCTCGGATGTGAC | |
| | TGGAGTTCAGACGTGTGCTCTTCCGATC | |
| Nova_i7_13 | CAAGCAGAAGACGGCATACGAGATTCTGTGCAGTGAC | |
| | TGGAGTTCAGACGTGTGCTCTTCCGATC | |
| Nova_i7_14 | CAAGCAGAAGACGGCATACGAGATTCTGCCATGTGAC | |
| | TGGAGTTCAGACGTGTGCTCTTCCGATC | |
| Nova_i7_15 | CAAGCAGAAGACGGCATACGAGATTCTGGAACGTGAC | |
| | TGGAGTTCAGACGTGTGCTCTTCCGATC | |
| Nova_i7_16 | CAAGCAGAAGACGGCATACGAGATTCTGAGGTGTGAC | |
| | TGGAGTTCAGACGTGTGCTCTTCCGATC | |
| SA160 | AATGATACGGCGACCACCGA | quantification |
| SA161 | CAAGCAGAAGACGGCATACGA | of NGS library |
| | | concentration |
| S_DMSfs1 | TTACCTCGTCTCTTGCTTCTTACCAGACCCAAACCAACT | universal |
| | CTCCTAGGCGAGCCCGGTCCGTAGCCA | foward primer |
| | | for FPPR DMS |
| | | library |
| | | construction |
| S_DMSrs | TTACCTCGTCTCTTAAATTTTTGGGCGCATATCAGATCC | universal |
| | | reverse primer |
| | | for Furin |
| | | cleavage site |
| | | DMS library |
| | | construction |
| S_DMS1 | TTCGTCTCTTGCTNNSTACCAGACCCAAACCAACTCTC | Furin cleavage |
| | CTAGGCGTGCCCGGTCCGTAGCCAGTCAAAGCATAATT | site_NNS |
| | GCGTACACCATGAG | primer |
| S_DMS2 | TTCGTCTCTTGCTTCTNNSCAGACCCAAACCAACTCTCC | |
| | TAGGCGTGCCCGGTCCGTAGCCAGTCAAAGCATAATTG | |
| | CGTACACCATGAG | |
| S_DMS3 | TTCGTCTCTTGCTTCTTACNNSACCCAAACCAACTCTCC | |
| | TAGGCGTGCCCGGTCCGTAGCCAGTCAAAGCATAATTG | |
| | CGTACACCATGAG | |
| S_DMS4 | TTCGTCTCTTGCTTCTTACCAGNNSCAAACCAACTCTCC | |
| | TAGGCGTGCCCGGTCCGTAGCCAGTCAAAGCATAATTG | |
| | CGTACACCATGAG | |
| S_DMS5 | TTCGTCTCTTGCTTCTTACCAGACCNNSACCAACTCTCC | |
| | TAGGCGTGCCCGGTCCGTAGCCAGTCAAAGCATAATTG | |
| | CGTACACCATGAG | |
| S_DMS6 | TTCGTCTCTTGCTTCTTACCAGACCCAANNSAACTCTCC | |
| | TAGGCGTGCCCGGTCCGTAGCCAGTCAAAGCATAATTG | |
| | CGTACACCATGAG | |
| S_DMS7 | TTCGTCTCTTGCTTCTTACCAGACCCAAACCNNSTCTCC | |
| | TAGGCGTGCCCGGTCCGTAGCCAGTCAAAGCATAATTG | |
| | CGTACACCATGAG | |
| S_DMS8 | TTCGTCTCTTGCTTCTTACCAGACCCAAACCAACNNSC | |
| | CTAGGCGTGCCCGGTCCGTAGCCAGTCAAAGCATAATT | |
| | GCGTACACCATGAG | |
| S_DMS9 | TTCGTCTCTTGCTTCTTACCAGACCCAAACCAACTCTN | |
| | NSAGGCGTGCCCGGTCCGTAGCCAGTCAAAGCATAATT | |
| | GCGTACACCATGAG | |
| S_DMS10 | TTCGTCTCTTGCTTCTTACCAGACCCAAACCAACTCTCC | |
| | TNNSCGTGCCCGGTCCGTAGCCAGTCAAAGCATAATTG | |
| | CGTACACCATGAG | |
| S_DMS11 | TTCGTCTCTTGCTTCTTACCAGACCCAAACCAACTCTCC | |
| | TAGGNNSGCCCGGTCCGTAGCCAGTCAAAGCATAATTG | |
| | CGTACACCATGAG | |
| S_DMS12 | TTCGTCTCTTGCTTCTTACCAGACCCAAACCAACTCTCC | |
| | TAGGCGTNNSCGGTCCGTAGCCAGTCAAAGCATAATTG | |
| | CGTACACCATGAG | |
| S_DMS13 | TTCGTCTCTTGCTTCTTACCAGACCCAAACCAACTCTCC | |
| | TAGGCGTGCCNNSTCCGTAGCCAGTCAAAGCATAATTG | |
| | CGTACACCATGAG | |
| S_DMS14 | TTCGTCTCTTGCTTCTTACCAGACCCAAACCAACTCTCC | |
| | TAGGCGTGCCCGGNNSGTAGCCAGTCAAAGCATAATT | |
| | GCGTACACCATGAG | |
| S_DMS15 | TTCGTCTCTTGCTTCTTACCAGACCCAAACCAACTCTCC | |
| | TAGGCGTGCCCGGTCCNNSGCCAGTCAAAGCATAATTG | |
| | CGTACACCATGAG | |
| S_DMS16 | TTCGTCTCTTGCTTCTTACCAGACCCAAACCAACTCTCC | |
| | TAGGCGTGCCCGGTCCGTANNSAGTCAAAGCATAATTG | |
| | CGTACACCATGAG | |
| S_DMS17 | TTCGTCTCTTGCTTCTTACCAGACCCAAACCAACTCTCC | |
| | TAGGCGTGCCCGGTCCGTAGCCNNSCAAAGCATAATTG | |
| | CGTACACCATGAG | |
| S_DMS18 | TTCGTCTCTTGCTTCTTACCAGACCCAAACCAACTCTCC | |
| | TAGGCGTGCCCGGTCCGTAGCCAGTNNSAGCATAATTG | |
| | CGTACACCATGAG | |
| S_DMS19 | TTCGTCTCTTGCTTCTTACCAGACCCAAACCAACTCTCC | |
| | TAGGCGTGCCCGGTCCGTAGCCAGTCAANNSATAATTG | |
| | CGTACACCATGAG | |
| S_DMS20 | TTCGTCTCTTAAASNNTTGGGCGCATATCAGATCCCGC | FPPR_NNS |
| | GCCGCGATGTCGCCCAAACAATCACCGTATTGTTTAAT | primer |
| | AAAGCCAGCATCGG | |
| S_DMS21 | TTCGTCTCTTAAATTTSNNGGCGCATATCAGATCCCGC | |
| | GCCGCGATGTCGCCCAAACAATCACCGTATTGTTTAAT | |
| | AAAGCCAGCATCGG | |
| S_DMS22 | TTCGTCTCTTAAATTTTTGSNNGCATATCAGATCCCGCG | |
| | CCGCGATGTCGCCCAAACAATCACCGTATTGTTTAATA | |
| | AAGCCAGCATCGG | |
| S_DMS23 | TTCGTCTCTTAAATTTTTGGGCSNNTATCAGATCCCGCG | |
| | CCGCGATGTCGCCCAAACAATCACCGTATTGTTTAATA | |
| | AAGCCAGCATCGG | |
| S_DMS24 | TTCGTCTCTTAAATTTTTGGGCGCASNNCAGATCCCGC | |
| | GCCGCGATGTCGCCCAAACAATCACCGTATTGTTTAAT | |
| | AAAGCCAGCATCGG | |
| S_DMS25 | TTCGTCTCTTAAATTTTTGGGCGCATATSNNATCCCGCG | |
| | CCGCGATGTCGCCCAAACAATCACCGTATTGTTTAATA | |
| | AAGCCAGCATCGG | |
| S_DMS26 | TTCGTCTCTTAAATTTTTGGGCGCATATCAGSNNCCGC | |
| | GCCGCGATGTCGCCCAAACAATCACCGTATTGTTTAAT | |
| | AAAGCCAGCATCGG | |
| S_DMS27 | TTCGTCTCTTAAATTTTTGGGCGCATATCAGATCSNNCG | |
| | CCGCGATGTCGCCCAAACAATCACCGTATTGTTTAATA | |
| | AAGCCAGCATCGG | |
| S_DMS28 | TTCGTCTCTTAAATTTTTGGGCGCATATCAGATCCCGSN | |
| | NCGCGATGTCGCCCAAACAATCACCGTATTGTTTAATA | |
| | AAGCCAGCATCGG | |
| S_DMS29 | TTCGTCTCTTAAATTTTTGGGCGCATATCAGATCCCGC | |
| | GCSNNGATGTCGCCCAAACAATCACCGTATTGTTTAAT | |
| | AAAGCCAGCATCGG | |
| S_DMS30 | TTCGTCTCTTAAATTTTTGGGCGCATATCAGATCCCGC | |
| | GCCGCSNNGTCGCCCAAACAATCACCGTATTGTTTAAT | |
| | AAAGCCAGCATCGG | |
| S_DMS31 | TTCGTCTCTTAAATTTTTGGGCGCATATCAGATCCCGC | |
| | GCCGCGATSNNGCCCAAACAATCACCGTATTGTTTAAT | |
| | AAAGCCAGCATCGG | |
| S_DMS32 | TTCGTCTCTTAAATTTTTGGGCGCATATCAGATCCCGC | |
| | GCCGCGATGTCSNNCAAACAATCACCGTATTGTTTAAT | |
| | AAAGCCAGCATCGG | |
| S_DMS33 | TTCGTCTCTTAAATTTTTGGGCGCATATCAGATCCCGC | |
| | GCCGCGATGTCGCCSNNACAATCACCGTATTGTTTAAT | |
| | AAAGCCAGCATCGG | |
| S_DMS34 | TTCGTCTCTTAAATTTTTGGGCGCATATCAGATCCCGC | |
| | GCCGCGATGTCGCCCAASNNATCACCGTATTGTTTAAT | |
| | AAAGCCAGCATCGG | |
| S_DMS35 | TTCGTCTCTTAAATTTTTGGGCGCATATCAGATCCCGC | |
| | GCCGCGATGTCGCCCAAACASNNACCGTATTGTTTAAT | |
| | AAAGCCAGCATCGG | |
| S_DMS36 | TTCGTCTCTTAAATTTTTGGGCGCATATCAGATCCCGC | |
| | GCCGCGATGTCGCCCAAACAATCSNNGTATTGTTTAAT | |
| | AAAGCCAGCATCGG | |
| S_DMS37 | TTCGTCTCTTAAATTTTTGGGCGCATATCAGATCCCGC | |
| | GCCGCGATGTCGCCCAAACAATCACCSNNTTGTTTAAT | |
| | AAAGCCAGCATCGG | |
| S_DMS38 | TTCGTCTCTTAAATTTTTGGGCGCATATCAGATCCCGC | |
| | GCCGCGATGTCGCCCAAACAATCACCGTASNNTTTAAT | |
| | AAAGCCAGCATCGG | |
| C508f | CACGACGCTCTTCCGATCTCATGTGAACAATTCATACG | 1st step PCR |
| | AATGTG | primers for |
| C509r | CAGACGTGTGCTCTTCCGATCTATTTGTTGGGATGGCA | amplication of |
| | ATGGAG | the Furin DMS |
| | | library |
| C512f | CACGACGCTCTTCCGATCTCGGACCCCAGTAAACCCTC | 1st step PCR |
| C513r | CAGACGTGTGCTCTTCCGATCTCCGAATGTCCATCCAG | primers for |
| | ACGTT | amplication of |
| | | the FPPR DMS |
| | | library |
| S_K854H | TTCGTCTCTTAAAGTGTTGGGCGCATATCAGATCCCGC | |
| | GCCGCGATGTCGCCCAAACAATCACCGTATTGTTTAAT | |
| | AAAGCCAGCATCGG | |
| S_A846W | TTCGTCTCTTAAATTTTTGGGCGCATATCAGATCCCGCC | |
| | ACGCGATGTCGCCCAAACAATCACCGTATTGTTTAATA | |
| | AAGCCAGCATCGG | |
| S_K854C | TTCGTCTCTTAAAGCATTGGGCGCATATCAGATCCCGC | |
| | GCCGCGATGTCGCCCAAACAATCACCGTATTGTTTAAT | |
| | AAAGCCAGCATCGG | |
| S_A852M | TTCGTCTCTTAAATTTTTGCATGCATATCAGATCCCGCG | |
| | CCGCGATGTCGCCCAAACAATCACCGTATTGTTTAATA | |
| | AAGCCAGCATCGG | |
| S_K854L | TTCGTCTCTTAAACAGTTGGGCGCATATCAGATCCCGC | |
| | GCCGCGATGTCGCCCAAACAATCACCGTATTGTTTAAT | |
| | AAAGCCAGCATCGG | |
| S_Q853V | TTCGTCTCTTAAATTTCACGGCGCATATCAGATCCCGC | |
| | GCCGCGATGTCGCCCAAACAATCACCGTATTGTTTAAT | |
| | AAAGCCAGCATCGG | |
| S_K854N | TTCGTCTCTTAAAGTTTTGGGCGCATATCAGATCCCGC | |
| | GCCGCGATGTCGCCCAAACAATCACCGTATTGTTTAAT | |
| | AAAGCCAGCATCGG | |
| S_D843M | TTCGTCTCTTAAATTTTTGGGCGCATATCAGATCCCGC | |
| | GCCGCGATCATGCCCAAACAATCACCGTATTGTTTAAT | |
| | AAAGCCAGCATCGG | |
| S_G838Y | TTCGTCTCTTAAATTTTTGGGCGCATATCAGATCCCGC | |
| | GCCGCGATGTCGCCCAAACAATCGTAGTATTGTTTAAT | |
| | AAAGCCAGCATCGG | |
| S_Y837F | TTCGTCTCTTAAATTTTTGGGCGCATATCAGATCCCGC | |
| | GCCGCGATGTCGCCCAAACAATCACCGAATTGTTTAAT | |
| | AAAGCCAGCATCGG | |
| S_K854W | TTCGTCTCTTAAACCATTGGGCGCATATCAGATCCCGC | |
| | GCCGCGATGTCGCCCAAACAATCACCGTATTGTTTAAT | |
| | AAAGCCAGCATCGG | |
| S_C840S | TTCGTCTCTTAAATTTTTGGGCGCATATCAGATCCCGC | |
| | GCCGCGATGTCGCCCAAGCTATCACCGTATTGTTTAAT | |
| | AAAGCCAGCATCGG | |
| S_D848N | TTCGTCTCTTAAATTTTTGGGCGCATATCAGGTTCCGCG | |
| | CCGCGATGTCGCCCAAACAATCACCGTATTGTTTAATA | |
| | AAGCCAGCATCGG | |
| S_C851S | TTCGTCTCTTAAATTTTTGGGCGCTTATCAGATCCCGCG | |
| | CCGCGATGTCGCCCAAACAATCACCGTATTGTTTAATA | |
| | AAGCCAGCATCGG | |
| S_P681Y | TTCGTCTCTTGCTTCTTACCAGACCCAAACCAACTCTTA | |
| | CAGGCGTGCCCGGTCCGTAGCCAGTCAAAGCATAATT | |
| | GCGTACACCATGAG | |
| S_R683H | TTCGTCTCTTGCTTCTTACCAGACCCAAACCAACTCTCC | |
| | TAGGCACGCCCGGTCCGTAGCCAGTCAAAGCATAATT | |
| | GCGTACACCATGAG | |
| S_A684H | TTCGTCTCTTGCTTCTTACCAGACCCAAACCAACTCTCC | |
| | TAGGCGACACCGGTCCGTAGCCAGTCAAAGCATAATT | |
| | GCGTACACCATGAG | |
| S_R685F | TTCGTCTCTTGCTTCTTACCAGACCCAAACCAACTCTCC | |
| | TAGGCGTGCCTTCTCCGTAGCCAGTCAAAGCATAATTG | |
| | CGTACACCATGAG | |
| S_V687I | TTCGTCTCTTGCTTCTTACCAGACCCAAACCAACTCTCC | |
| | TAGGCGTGCCCGGTCCATCGCCAGTCAAAGCATAATTG | |
| | CGTACACCATGAG | |
| S_A688T | TTCGTCTCTTGCTTCTTACCAGACCCAAACCAACTCTCC | |
| | TAGGCGTGCCCGGTCCGTAACCAGTCAAAGCATAATTG | |
| | CGTACACCATGAG | |
| O_K854H | TTCGTCTCTTGAAGTGTTGAGCACAGATAAGGTCGC | |
| O_A846W | TTCGTCTCTTGAATTTTTGAGCACAGATAAGGTCGCGC | |
| | CACGCAATGTCTCCCAAACAATC | |
| O_C840S | TTCGTCTCTTGAATTTTTGAGCACAGATAAGGTCGCGG | |
| | GCCGCAATGTCTCCCAAGCTATCCCCATACTGCTTAAT | |
| | GAAAC | |
| O_D848N | TTCGTCTCTTGAATTTTTGAGCACAGATAAGGTTGCGG | |
| | GCCGCAATGTCT | |
| O_C851S | TTCGTCTCTTGAATTTTTGAGCGCTGATAAGGTCGCGG | |
| | GCCG | |
| BW588 | GTCTTTCTCGCTACCTGGTACG | qRT-PCR |
| BW589 | GGTCCTGAGTCTCAGACAAAGC | primer for |
| RdRp-F | CGCATACAGTCTTRCAGGCT | CHC, RdRp, |
| RdRp-R | GTGTGATGTTGAWATGACATGGTC | GAPDH(human), |
| probe | FAM-TTAAGATGTGGTGCTTGCATACGTAGAC-IABKFQ | and ß-actin |
| GAPDH-F | ATTCCACCCATGGCAAATTC | (hamster) |
| GAPDH-R | CGCTCCTGGAAGATGGTGAT | |
| ß-actin- | ACGGCCAGGTCATCACTATTG | |
| F(hamster) | | |
| ß-actin- | CAAGAAGGAAGGCTGGAAAAG | |
| R(hamster) | | |

Supplementary Table 3 discloses SEQ ID NOS 116-137, 119, 121, 138, 135, and 139-142, respectively, in order of appearance.

Supplementary Table 3

| sgRNA list | | |
|---|---|---|
| sgRNA ID | sgRNA sequence | Description |
| ACE2_sgRNA1 | ACAGTTTAGACTACAATGAG | sgRNA used in A549-ACE2 |
| ACE2_sgRNA3 | ATCTTCAATCAACTGGCCG | cells |
| CAB39_sgRNA1 | TAATAGGGCAAAAAAGACG | |
| CAB39_sgRNA2 | AGCTGTAAATCAGCTACCA | |
| AP2M1_sgRNA1 | ACCCACAGAATTCCGAGAC | |
| AP2M1_sgRNA2 | TACTTTGGCAAGATCAGCG | |
| FCHO2_sgRNA1 | TTTAACAGAGCTACCATAG | |
| FCHO2_sgRNA2 | CTAGTTTTGTCATTGACC | |
| GBP6_sgRNA1 | CTTCTAGCCAATATTGAGA | |
| GBP6_sgRNA2 | AAATTGGATGTTTGAACTC | |
| RNF2_sgRNA1 | GAGTTACAACGAACACCTC | |
| RNF2_sgRNA2 | ATTCACTGTGTAGACTTCG | |
| ZEB1_sgRNA1 | TGAATGGGCGACCAAGAAC | |
| ZEB1_sgRNA2 | TACTTAAAGTGGCGGTAGA | |
| UBIAD1_sgRNA1 | CCGTGGGATCTGTAGGCAA | |
| UBIAD1_sgRNA2 | CACTCACACCGGTGGCCCT | |
| NDUFB10_sgRNA1 | AGTGATGTCTGGCACGCGG | |
| NDUFB10_sgRNA2 | GACCCGTGACCCTCGTGAG | |
| safe harbor_sgRNA1 | CCATTCAGTCTGAAATACGG | |
| non targeting_sgRNA1 | CCTCATCCACGAAAGATGC | |
| non targeting_sgRNA2 | TACGCTGCGACACCCGGTG | |
| ACE2_sgRNA | AGAGTTTAGACTACAATGAG | sgRNA used in Vero E6- |
| CAB39_sgRNA | AGCTGTAAATCAGCTACCA | TMPRSS2 cells |
| AP2M1_sgRNA | TACTTTGGCAAGATCAGCG | |
| FCHO2_sgRNA | CAAGAGATGGTCCAAATAGG | |
| non targeting_sgRNA | CCTCATCCACGAAAGATGC | |
| shRNA list | | |
| shRNA ID | target sequence | |
| CHC-sh1 | GCAGAAGAATCAACGTTATTT | shRNA used in A549-ACE2 |
| CHC-sh2 | CTGGAGGGAAGTTACATATTA | cells |
| CHC-sh3 | GGAGGGAAGTTACATATTATT | |
| ctr-shRNA | CGAGGGCGACTTAACCTTAGG | |

Exemplary products, systems and methods are set out in the following items:

1. A high throughput screening method for identifying cell fusion or syncytium formation potential of a viral spike protein, the method comprising the step of
(a) preparing a plurality of spike protein variants;
(b) incorporating said spike protein variants into a plurality of spike-presenting sender cells, each cell comprising a spike protein variant from said plurality of spike protein variants;
(c) contacting said spike-presenting sender cells with a plurality of receiver cells expressing angiotensin-converting enzyme 2 (ACE2) to obtain a mixture;
(d) incubating the mixture obtained in step (c) in a microfluidic system comprising multiple droplets, each droplet comprising an individual sender cell and an individual receiver cell; and
(e) determining the syncytium-forming potential of the spike protein variants in the droplets.

2. The method of item 1, wherein the viral spike protein is a respiratory virus spike protein.

3. The method of any one of the preceding items, wherein the respiratory virus is a coronavirus.

4. The method of any one of the preceding items, wherein the respiratory virus is selected from the group consisting of severe acute respiratory syndrome (SARS) coronavirus (SARS-COV), SARS-COV-2 (COVID-19), Middle East Respiratory Syndrome (MERS), respiratory syncytial virus (RSV), influenza virus, parainfluenza virus (PIV), pneumovirus (PMV), metapneumovirus (MPV), respirovirus, and rubulavirus.

5. The method of any one of the preceding items, wherein the respiratory virus is SARS-CoV-2 (COVID-19).

6. The method of any one of the preceding items, wherein the plurality of spike protein variants is prepared by site saturation mutagenesis (SSM).

7. The method of any one of the preceding items, wherein the respiratory virus is SARS-CoV-2, and wherein the SSM is over the fusion peptide proximal region (FPPR) of SARS-CoV-2.

8. The method of any one of the preceding items, wherein the respiratory virus is SARS-CoV-2, and wherein the SSM is over the furin cleavage-site region of SARS-COV-2.

9. The method of any one of the preceding items, wherein the syncytium-forming potential of the spike protein variants is determined by measuring a signal generated upon fusion of the sender and receiver cells.

10. The method any one of the preceding items, wherein the signal is a fluorescent, bioluminescent, chemiluminescent or a radioactive signal.

11. The method any one of the preceding items, wherein the signal is a green fluorescent protein (GFP) signal that is generated upon fusion of the sender and receiver cells.

12. The method of any one of the preceding items, wherein the syncytium-forming potential of the spike protein variants is determined using deep mutational scanning (DMS) in the spike-presenting sender cells.

13. The method of any one of the preceding items, further comprising the step of enriching the spike variants having enhanced syncytium-formation potential.

14. The method of any one of the preceding items, further comprising the step of analyzing the abundance of each variant by sequencing spike variants having enhanced syncytium-formation potential.

15. A high throughput screening method for identifying cell fusion or syncytium formation potential of a viral spike protein, the method comprising the steps of:
(a) preparing a plurality of spike protein variants;
(b) incorporating said spike protein variants into a plurality of spike-presenting sender cells, each cell comprising a spike protein variant from said plurality of spike protein variants;
(c) contacting said spike-presenting sender cells with a plurality of receiver cells expressing angiotensin-converting enzyme 2 (ACE2) to obtain a mixture;
(d) performing size-exclusion selection by passing the mixture through a cell strainer; and
(e) determining the syncytium-forming potential of the spike protein variants.

16. The method of item 15, further comprising the step of collecting a first syncytia population that is retained on said strainer, and a second syncytia population that passes through the strainer.

17. The method of item 16, further comprising the step of enriching the spike variants having enhanced syncytium-formation potential from the first and second syncytia populations.

18. The method of item 16, further comprising the step of analyzing the abundance of each variant by sequencing spike variants having enhanced syncytium-formation potential in the first and second syncytia populations.

19. The method of any one of items 15-18, wherein the viral spike protein is a respiratory virus spike protein.

20. The method of item 19, wherein the respiratory virus is a coronavirus.

21. The method of any one of items 19-20, wherein the respiratory virus is selected from the group consisting of severe acute respiratory syndrome (SARS) coronavirus (SARS-COV), SARS-COV-2 (COVID-19), Middle East Respiratory Syndrome (MERS), respiratory syncytial virus (RSV), influenza virus, parainfluenza virus (PIV), pneumovirus (PMV), metapneumovirus (MPV), respirovirus, and rubulavirus.

22. The method of any one of items 19-21, wherein the respiratory virus is SARS-COV-2 (COVID-19).

23. The method of any one of items 15-22, wherein the plurality of spike protein variants is prepared by site saturation mutagenesis (SSM).

24. The method of item 23, wherein the respiratory virus is SARS-COV-2, and wherein the saturation mutagenesis is over the fusion peptide proximal region (FPPR) of SARS-COV-2.

25. The method of item 23, wherein the respiratory virus is SARS-COV-2, and wherein the saturation mutagenesis is over the furin cleavage-site region of SARS-COV-2.

26. The method of any one of items 15-25, wherein the syncytium-forming potential of the spike protein variants is determined by measuring a signal generated upon fusion of the sender and receiver cells.

27. The method item 26, wherein the signal is a fluorescent, bioluminescent, chemiluminescent or a radioactive signal.

28. The method item 26, wherein the signal is a green fluorescent protein (GFP) signal that is generated upon fusion of the sender and receiver cells.

29. The method of any one of items 15-28, wherein the syncytium-forming potential of the spike protein variants is determined using deep mutational scanning (DMS) in the spike-presenting sender cells.

30. A method for identifying a cellular factor that promotes spike protein-induced syncytium formation, the method comprising the steps of:
(a) preparing a plurality of spike protein variants;
(b) incorporating said spike protein variants into a plurality of spike-presenting sender cells, each cell comprising a spike protein variant from said plurality of spike protein variants;
(c) contacting said spike-presenting sender cells with a plurality of receiver cells expressing angiotensin-converting enzyme 2 (ACE2) and single guide RNA (sgRNA)-Cas9 to obtain a mixture;
(d) isolating unfused receiver cells from the mixture;
(e) comparing the sgRNA abundance in unmixed receiver cells and isolated unfused receiver cells from step (d); and
(f) identifying a factor in the receiver cell that promotes syncytium formation.

31. The method of item 30, wherein the viral spike protein is a respiratory virus spike protein.

32. The method of any one of items 30-31, wherein the respiratory virus is a coronavirus.

33. The method of any one of items 30-32, wherein the respiratory virus is selected from the group consisting of severe acute respiratory syndrome (SARS) coronavirus (SARS-COV), SARS-COV-2 (COVID-19), Middle East Respiratory Syndrome (MERS), respiratory syncytial virus (RSV), influenza virus, parainfluenza virus (PIV), pneumovirus (PMV), metapneumovirus (MPV), respirovirus, and rubulavirus.

34. The method of any one of items 31-33, wherein the respiratory virus is SARS-COV-2 (COVID-19).

35. The method of any one of items 30-34, wherein the plurality of spike protein variants is prepared by site saturation mutagenesis (SSM).

36. The method of item 35, wherein the respiratory virus is SARS-COV-2, and wherein the saturation mutagenesis is over the fusion peptide proximal region (FPPR) of SARS-COV-2.

37. The method of item 35, wherein the respiratory virus is SARS-COV-2, and wherein the saturation mutagenesis is over the furin cleavage-site region of SARS-COV-2.

38. The method of any one of items 30-37, wherein the syncytium-forming potential of the spike protein variants is determined by measuring a signal generated upon fusion of the sender and receiver cells.

39. The method item 38, wherein the signal is a fluorescent, bioluminescent, chemiluminescent or a radioactive signal.

40. The method item 38, wherein the signal is a green fluorescent protein (GFP) signal that is generated upon fusion of the sender and receiver cells.

41. The method of any one of items 30-40, wherein the host factor comprises a core regulator of clathrin-mediated endocytosis (CME).

42. The method of item 41, wherein the CME regulator is selected from the group consisting of FCHO2, AP2M1, CAB39, RNF2 and GBP6.

43. A high throughput screening method for identifying cell fusion or syncytium formation potential of a first protein and a second protein in a biological system, the method comprising the steps of:
(a) preparing a plurality of first protein variants;
(b) incorporating said first protein variants into a plurality of first protein-presenting cells, each cell comprising a first protein variant from said plurality of first protein variants;
(c) contacting said first protein presenting cells with a plurality of cells expressing said second protein to obtain a mixture;
(d) incubating the mixture obtained in step (c) in a microfluidic system comprising multiple droplets, each droplet comprising an individual first protein presenting cell and an individual cell expressing said second protein; and
(e) determining the syncytium-forming potential of the first protein variants in the droplets.

44. The method of item 43, wherein the biological system is a virus, tumor, maternal-fetal material exchange in the placenta, muscle contraction and bone resorption.

45. The method of item 44 wherein the virus is selected from the group consisting of human immunodeficiency virus, Herpesviridae, respiratory syncytial virus and Coronaviridae.

46. The method of any one of the items 43 to 45, wherein the first protein is from a tumor and the second protein is from normal somatic cells or dendritic cells.

47. The method of any one of items 43 to 46, wherein the first protein or second protein is from multinucleated cells selected from the group consisting of syncytiotrophoblasts, myotybes and osteoclasts.

48. The method of any one of items 43 to 47, wherein the first protein is from B cells and the second protein is from myeloma cells that produces hydridoma.

49. The method of any one of items 43 to 48, wherein the first protein is from human embryonic stem cells and the second protein is from somatic cells.

50. The method of any one of items 43 to 49, wherein the syncytium-forming potential of the first protein variants is determined by measuring a signal generated upon fusion of the first protein presenting cells and cells expressing said second protein.

51. The method of any one of items 43 to 50, wherein the signal is a fluorescent, bioluminescent, chemiluminescent or a radioactive signal.

52. The method of any one of items 43 to 51, wherein the signal is a green fluorescent protein (GFP) signal that is generated upon fusion of the first protein presenting cells and cells expressing said second protein.

53. The method of any one of items 43 to 52, wherein the syncytium-forming potential of the first protein variants is determined using deep mutational scanning (DMS) in the first protein-presenting cells.

54. The method of item 43, further comprising the step of enriching the first protein variants having enhanced syncytium-formation potential.

55. The method of any one of items 43 and 54, further comprising the step of analyzing the abundance of each variant by sequencing first protein variants having enhanced syncytium-formation potential.

56. The method of one of items 43, 54 and 55, wherein the mixture in step (c) further comprises single guide RNA (sgRNA)-Cas9, and further comprising the steps of (f) isolating unfused cells expressing said second protein from the mixture; (g) comparing the sgRNA abundance in unmixed cells expressing said second protein and the isolated unfused cells expressing said second protein from step (d).

57. A high throughput screening method for identifying cell fusion or syncytium formation potential of a first protein and a second protein in a biological system, the method comprising the step of
(a) preparing a plurality of first protein variants;
(b) incorporating said first protein variants into a plurality of first protein-presenting cells, each cell comprising a first protein variant from said plurality of first protein variants;
(c) contacting said first-presenting cells with a plurality of cells expressing a second protein to obtain a mixture;
(d) performing size-exclusion selection by passing the mixture through a cell strainer; and
(e) determining the syncytium-forming potential of the first protein variants.

58. The method of item 57, further comprising the step of collecting a first syncytia population that is retained on said strainer, and a second syncytia population that passes through the strainer.

59. The method of any one of items 57 and 58, further comprising the step of enriching the first protein variants having enhanced syncytium-formation potential from the first and second syncytia populations.

60. The method of item 59, further comprising the step of analyzing the abundance of each variant by sequencing the first protein variants having enhanced syncytium-formation potential in the first and second syncytia populations.

61. The method of any one of items 57 to 60, wherein the plurality of the first protein variants is prepared by site saturation mutagenesis (SSM).

62. The method of any one of items 57 to 61, wherein the syncytium-forming potential of the first protein variants is determined by measuring a signal generated upon fusion of the first protein presenting cells and cells expressing said second protein.

63. The method of any one of items 57 to 62, wherein the signal is a fluorescent, bioluminescent, chemiluminescent or a radioactive signal.

64. The method of any one of items 57 to 63, wherein the signal is a green fluorescent protein (GFP) signal that is generated upon fusion of the first protein presenting cells and cells expressing said second protein.

65. The method of item 57 wherein the mixture in step (c) further comprises single guide RNA (sgRNA)-Cas9, and further comprising the steps of (f) isolating unfused cells expressing said second protein from the mixture; (g) comparing the sgRNA abundance in unmixed cells expressing said second protein and the isolated unfused cells expressing said second protein.

66. The method of item 57 wherein the syncytium-forming potential of the first protein variants is determined using deep mutational scanning (DMS) in the first protein-presenting cells.

67. A method of inhibiting or suppressing syncytium formation induced by a viral spike protein, the method comprising the step of administering to a viral cell comprising said spike protein an inhibitor of CME inhibitor.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

1. Yan, R. et al. Structural basis for the recognition of SARS-COV-2 by full-length human ACE2. Science 367, 1444-1448 (2020).
2. Hoff M., Kleine-Weber, H. & Pohlmann, S. A multibasic cleavage site in the spike protein of SARS-COV-2 is essential for infection of human lung cells. Mol. Cell 78, 779-784.e5 (2020).
3. Buchrieser, J. et al. Syncytia formation by SARS-COV-2-infected cells. EMBO J. 39, e106267 (2020).
4. Bussani, R. et al. Persistence of viral RNA, pneumocyte syncytia and thrombosis are hallmarks of advanced COVID-19 pathology. Ebiomedicine 61, 103104 (2020).
5. Zhang, Z. et al. SARS-COV-2 spike protein dictates syncytium-mediated lymphocyte elimination. Cell Death Differ. 28, 2765-2777 (2021).
6. Rajah, M. M., Bernier, A., Buchrieser, J. & Schwartz, O. The mechanism and consequences of SARS-COV-2 spike-mediated fusion and syncytia formation. J. Mol. Biol. 434, 167280 (2022).
7. Zeng, C. et al. SARS-COV-2 spreads through cell-to-cell transmission. Proc. Natl Acad. Sci. USA 119, e2111400119 (2022).
8. Cai, Y. et al. Structural basis for enhanced infectivity and immune evasion of SARS-COV-2 variants. Science 373, 642-648 (2021).
9. Jackson, C. B., Farzan, M., Chen, B. & Choe, H. Mechanisms of SARS-COV-2 entry into cells. Nat. Rev. Mol. Cell Biol. 23, 3-20 (2022).
10. Harvey, W. T. et al. SARS-COV-2 variants, spike mutations and immune escape. Nat. Rev. Microbiol. 19, 409-424 (2021).
11. Ord, M., Faustova, I. & Loog, M. The sequence at Spike S1/S2 site enables cleavage by furin and phospho-regulation in SARS-COV2 but not in SARS-COV1 or MERS-COV. Sci. Rep. 10, 16944 (2020).
12. Rajah, M. M. et al. SARS-COV-2 Alpha, Beta, and Delta variants display enhanced Spike-mediated syncytia formation. EMBO J. 40, e108944 (2021).
13. Saito, A. et al. Enhanced fusogenicity and pathogenicity of SARS-COV-2 Delta P681R mutation. Nature 602, 300-306 (2022).
14. Mohammad, A., Abubaker, J. & Al-Mulla, F. Structural modelling of SARS-COV-2 alpha variant (B.1.1.7) suggests enhanced furin binding and infectivity. Virus Res. 303, 198522 (2021).
15. Meng, B. et al. Altered TMPRSS2 usage by SARS-COV-2 Omicron impacts infectivity and fusogenicity. Nature 603, 706-714 (2022).
16. Willett, B. J. et al. SARS-COV-2 Omicron is an immune escape variant with an altered cell entry pathway. Nat. Microbiol. 7, 1161-1179 (2022).
17. Chan, J. F. et al. Virological features and pathogenicity of SARS-COV-2 Omicron BA.2. Cell Rep. Med. 3, 100743 (2022).
18. Madrigal, J. L. et al. Characterizing cell interactions at scale with made-to-order droplet ensembles (MODEs). Proc. Natl Acad. Sci. USA 119, e2110867119 (2022).
19. Braga, L. et al. Drugs that inhibit TMEM16 proteins block SARS-COV-2 spike-induced syncytia. Nature 594, 88 (2021).
20. Sanders, D. W. et al. SARS-COV-2 requires cholesterol for viral entry and pathological syncytia formation. Elife 10, e65962 (2021).
21. Merten, C. A. et al. Fusoselect: cell-cell fusion activity engineered by directed evolution of a retroviral glycoprotein. Nucleic Acids Res. 34, e41 (2006).
22. Wei, J. et al. Genome-wide CRISPR screens reveal host factors critical for SARS-CoV-2 infection. Cell 184, 76-91.e13 (2021).
23. Daniloski, Z. et al. Identification of required host factors for SARS-COV-2 infection in human cells. Cell 184, 92-105.e16 (2021).
24. Baggen, J. et al. Genome-wide CRISPR screening identifies TMEM106B as a proviral host factor for SARS-COV-2. Nat. Genet. 53, 435-444 (2021).
25. Grodzki, M. et al. Genome-scale CRISPR screens identify host factors that promote human coronavirus infection. Genome Med. 14, 10 (2022).
26. Rebendenne, A. et al. Bidirectional genome-wide CRISPR screens reveal host factors regulating SARS-COV-2, MERS-COV and seasonal HCoVs. Nat. Genet. 54, 1090-1102 (2022).
27. Biering, S. B. et al. Genome-wide bidirectional CRISPR screens identify mucins as host factors modulating SARS-COV-2 infection. Nat. Genet. 54, 1078-1089 (2022).
28. Israeli, M. et al. Genome-wide CRISPR screens identify GATA6 as a proviral host factor for SARS-COV-2 via modulation of ACE2. Nat. Commun. 13, 2237 (2022).
29. Cabantous, S., Terwilliger, T. C. & Waldo, G. S. Protein tagging and detection with engineered self-assembling fragments of green fluorescent protein. Nat. Biotechnol. 23, 102-107 (2005).
30. Ma, D. et al. Expression of SARS-COV-2 receptor ACE2 and TMPRSS2 in human primary conjunctival and pterygium cell lines and in mouse cornea. Eye 34, 1212-1219 (2020).
31. Cai, Y. et al. Distinct conformational states of SARS-COV-2 spike protein. Science 369, 1586-1592 (2020).
32. Wright, D. W. et al. Tracking SARS-COV-2 mutations and variants through the COG-UK-Mutation Explorer. Virus Evol. 8, veac023 (2022).
33. Daniloski, Z. et al. The Spike D614G mutation increases SARS-COV-2 infection of multiple human cell types. Elife 10, e65365 (2021).
34. Zhang, L. et al. SARS-COV-2 spike-protein D614G mutation increases virion spike density and infectivity. Nat. Commun. 11, 6013 (2020).
35. Korber, B. et al. Tracking changes in SARS-COV-2 spike: evidence that D614G increases infectivity of the COVID-19 virus. Cell 182, 812-827 e819 (2020).
36. Zhang, J. et al. Structural impact on SARS-COV-2 spike protein by D614G substitution. Science 372, 525-530 (2021).
37. Xiong, X. et al. A thermostable, closed SARS-COV-2 spike protein trimer. Nat. Struct. Mol. Biol. 27, 934-941 (2020).
38. Benton, D. J. et al. The effect of the D614G substitution on the structure of the spike glycoprotein of SARS-COV-2. Proc. Natl Acad. Sci. USA 118, e2022586118 (2021).
39. Ozono, S. et al. SARS-COV-2 D614G spike mutation increases entry eff y with enhanced ACE2-binding aff. Nat. Commun. 12, 848 (2021).
40. Johnson, B. A. et al. Loss of furin cleavage site attenuates SARS-COV-2 pathogenesis. Nature 591, 293-299 (2021).
41. Peacock, T. P. et al. The furin cleavage site in the SARS-COV-2 spike protein is required for transmission in ferrets. Nat. Microbiol. 6, 899-909 (2021).
42. Matthews, D. J., Goodman, L. J., Gorman, C. M. & Wells, J. A. A survey of furin substrate specificity using substrate phage display. Protein Sci. 3, 1197-1205 (1994).
43. Gonzalez-Reiche, A. S. et al. Sequential intrahost evolution and onward transmission of SARS-COV-2 variants. Nat. Commun. 14, 3235 (2023).
44. Goncalves, E. et al. Minimal genome-wide human CRISPR-Cas9 library. Genome Biol. 22, 40 (2021).
45. Li, W. et al. MAGeCK enables robust identification of essential genes from genome-scale CRISPR/Cas9 knockout screens. Genome Biol. 15, 554 (2014).
46. Allen, F. et al. JACKS: joint analysis of CRISPR/Cas9 knockout screens. Genome Res. 29, 464-471 (2019).
47. Koch, J. et al. TMPRSS2 expression dictates the entry route used by SARS-COV-2 to infect host cells. EMBO J. 40, e107821 (2021).
48. Henne, W. M. et al. FCHo proteins are nucleators of clathrin-mediated endocytosis. Science 328, 1281-1284 (2010).
49. Helbig, I. et al. A recurrent missense variant in AP2M1 impairs clathrin-mediated endocytosis and causes developmental and epileptic encephalopathy. Am. J. Hum. Genet. 104, 1060-1072 (2019).
50. Batchelder, E. M. & Yarar, D. Differential requirements for clathrin-dependent endocytosis at sites of cell-substrate adhesion. Mol. Biol. Cell 21, 3070-3079 (2010).
51. Shin, N. Y. et al. Dynamin and endocytosis are required for the fusion of osteoclasts and myoblasts. J. Cell Biol. 207, 73-89 (2014).
52. Greaney, A. J. et al. Comprehensive mapping of mutations in the SARS-COV-2 receptor-binding domain that affect recognition by polyclonal human plasma antibodies. Cell Host Microbe 29, 463-476.e6 (2021).
53. Greaney, A. J. et al. Mapping mutations to the SARS-COV-2 RBD that escape binding by different classes of antibodies. Nat. Commun. 12, 4196 (2021).
54. Greaney, A. J. et al. Complete mapping of mutations to the SARS-COV-2 spike receptor-binding domain that escape antibody recognition. Cell Host Microbe 29, 44-57.e9 (2021).
55. Starr, T. N., Greaney, A. J., Dingens, A. S. & Bloom, J. D. Complete map of SARS-COV-2 RBD mutations that escape the monoclonal antibody LY-CoV555 and its cocktail with LY-CoV016. Cell Rep. Med. 2, 100255 (2021).
56. Starr, T. N. et al. Shifting mutational constraints in the SARS-COV-2 receptor-binding domain during viral evolution. Science 377, 420-424 (2022).
57. Starr, T. N. et al. Deep mutational scanning of SARS-COV-2 receptor binding domain reveals constraints on folding and ACE2 binding. Cell 182, 1295-1310.e20 (2020).
58. Garrett, M. E. et al. High-resolution profiling of pathways of escape for SARS-CoV-2 spike-binding antibodies. Cell 184, 2927-2938.e11 (2021).
59. Taft, J. M. et al. Deep mutational learning predicts ACE2 binding and antibody escape to combinatorial mutations in the SARS-COV-2 receptor-binding domain. Cell 185, 4008-4022.e14 (2022).
60. Choi, G. C. G. et al. Combinatorial mutagenesis en masse optimizes the genome editing activities of SpCas9. Nat. Methods 16, 722-730 (2019).
61. Vercauteren, D. et al. The use of inhibitors to study endocytic pathways of gene carriers: optimization and pitfalls. Mol. Ther. 18, 561-569 (2010).
62. Plaze, M. et al. Repurposing chlorpromazine to treat COVID-19: the reCo Very study. Encephale 46, 169-172 (2020).
63. Reis, G. et al. Effect of early treatment with fluvoxamine on risk of emergency care and hospitalisation among patients with COVID-19: the TOGETHER randomised, platform clinical trial. Lancet Glob. Health 10, e42-e51 (2022).
64. Lifson, J. D. et al. Induction of CD4-dependent cell fusion by the HTLV-III/LAV envelope glycoprotein. Nature 323, 725-728 (1986).
65. Hoggan, M. D. & Roizman, B. The isolation and properties of a variant of Herpes simplex producing multinucleated giant cells in monolayer cultures in the presence of antibody. Am. J. Hyg. 70, 208-219 (1959).
66. Chanock, R., Roizman, B. & Myers, R. Recovery from infants with respiratory illness of a virus related to chimpanzee coryza agent (CCA). I. Isolation, properties and characterization. Am. J. Hyg. 66, 281-290 (1957).
67. Chen, E. H. & Olson, E. N. Unveiling the mechanisms of cell-cell fusion. Science 308, 369-373 (2005).
68. Brukman, N. G., Uygur, B., Podbilewicz, B. & Chernomordik, L. V. How cells fuse. J. Cell Biol. 218, 1436-1451 (2019).
69. Kohler, G. & Milstein, C. Continuous cultures of fused cells secreting antibody of predefined specificity. Nature 256, 495-497 (1975).
70. Yamanaka, S. & Blau, H. M. Nuclear reprogramming to a pluripotent state by three approaches. Nature 465, 704-712 (2010).
71. Zhou, P., Wan, Y. K., Chan, B. K. C., Choi, G. C. G. & Wong, A. S. L. Extensible combinatorial CRISPR screening in mammalian cells. STAR Protoc. 2, 100255 (2021).
72. Faure, A. J., Schmiedel, J. M., Baeza-Centurion, P. & Lehner, B. DiMSum: an error model and pipeline for analyzing deep mutational scanning data and diagnosing common experimental pathologies. Genome Biol. 21, 207 (2020).
73. Bray, N. L., Pimentel, H., Melsted, P. & Pachter, L. Near-optimal probabilistic RNA-seq quantification. Nat. Biotechnol. 34, 525-527 (2016).
74. Soneson, C., Love, M. I. & Robinson, M. D. Differential analyses for RNA-seq: transcript-level estimates improve gene-level inferences. F1000Research 4, 1521 (2015).
75. Love, M. I., Huber, W. & Anders, S. Moderated estimation of fold change and dispersion for RNA-seq data with DESeq2. Genome Biol. 15, 550 (2014).
76. Chan, J. F. et al. Altered host protease determinants for SARS-COV-2 Omicron. Sci. Adv. 9, eadd3867 (2023).
77. Rodriguez-Rivas, J., Croce, G., Muscat, M. & Weigt, M. Epistatic models predict mutable sites in SARS-COV-2 proteins and epitopes. Proc. Natl Acad. Sci. USA 119, e2113118119 (2022).
78. Li, W., Jaroszewski, L. & Godzik, A. Clustering of highly homologous sequences to reduce the size of large protein databases. Bioinformatics 17, 282-283 (2001).
79. Chan, C. W. F., Wang, B., Nan, L. et al. High-throughput screening of genetic and cellular drivers of syncytium formation induced by the spike protein of SARS-CoV-2. Nat. Biomed. Eng (2023). https://doi.org/10.1038/s41551-023-01140-z.

What is claimed:

1. A high throughput screening method for identifying cell fusion or syncytium formation potential of a viral spike protein, the method comprising the step of
(a) preparing a plurality of spike protein variants;
(b) incorporating said spike protein variants into a plurality of spike-presenting sender cells, each cell comprising a spike protein variant from said plurality of spike protein variants;
(c) contacting said spike-presenting sender cells with a plurality of receiver cells expressing angiotensin-converting enzyme 2 (ACE2) to obtain a mixture;
(d) incubating the mixture obtained in step (c) in a microfluidic system comprising multiple droplets, each droplet comprising an individual sender cell and an individual receiver cell; and
(e) determining the syncytium-forming potential of the spike protein variants in the droplets.

2. The method of claim 1, wherein the viral spike protein is a SARS-COV-2 (COVID-19) spike protein.

3. The method of claim 1, wherein the plurality of spike protein variants is prepared by site saturation mutagenesis (SSM).

4. The method of claim 3, wherein the SSM is over the fusion peptide proximal region (FPPR) or the furin cleavage site of SARS-COV-2.

5. The method of claim 1, wherein the syncytium-forming potential of the spike protein variants is determined by measuring a signal generated upon fusion of the sender and receiver cells.

6. The method claim 5, wherein the signal is a fluorescent, bioluminescent, chemiluminescent or a radioactive signal.

7. The method claim 5, wherein the signal is a green fluorescent protein (GFP) signal that is generated upon fusion of the sender and receiver cells.

8. The method of claim 1, wherein the syncytium-forming potential of the spike protein variants is determined using deep mutational scanning (DMS) in the spike-presenting sender cells.

9. The method of claim 1, further comprising the step of enriching the spike variants having enhanced syncytium-formation potential.

10. The method of claim 1, further comprising the step of analyzing the abundance of each variant by sequencing spike variants having enhanced syncytium-formation potential.

11. A high throughput screening method for identifying cell fusion or syncytium formation potential of a viral spike protein, the method comprising the step of
(a) preparing a plurality of spike protein variants;
(b) incorporating said spike protein variants into a plurality of spike-presenting sender cells, each cell comprising a spike protein variant from said plurality of spike protein variants;
(c) contacting said spike-presenting sender cells with a plurality of receiver cells expressing angiotensin-converting enzyme 2 (ACE2) to obtain a mixture;
(d) performing size-exclusion selection by passing the mixture through a cell strainer; and
(e) determining the syncytium-forming potential of the spike protein variants.

12. The method of claim 11, further comprising the step of collecting a first syncytia population that is retained on said strainer, and a second syncytia population that passes through the strainer.

13. The method of claim 12, further comprising the step of enriching the spike variants having enhanced syncytium-formation potential from the first and second syncytia populations.

14. The method of claim 12, further comprising the step of analyzing the abundance of each variant by sequencing spike variants having enhanced syncytium-formation potential in the first and second syncytia populations.

15. The method of claim 11, wherein the plurality of spike protein variants is prepared by site saturation mutagenesis (SSM).

16. The method of claim 15, wherein the SSM is over the fusion peptide proximal region (FPPR) or the furin cleavage site of SARS-COV-2.

17. The method of claim 11, wherein the syncytium-forming potential of the spike protein variants is determined by measuring a signal generated upon fusion of the sender and receiver cells.

18. The method of claim 17, wherein the signal is a fluorescent, bioluminescent, chemiluminescent or a radioactive signal.

19. The method of claim 17, wherein the signal is a green fluorescent protein (GFP) signal that is generated upon fusion of the sender and receiver cells.

20. The method of claim 11, wherein the syncytium-forming potential of the spike protein variants is determined using deep mutational scanning (DMS) in the spike-presenting sender cells.

21. A method for identifying a cellular factor that promotes viral spike protein-induced syncytium formation, the method comprising the steps of:
(a) preparing a plurality of spike protein variants;
(b) incorporating said spike protein variants into a plurality of spike-presenting sender cells, each cell comprising a spike protein variant from said plurality of spike protein variants;
(c) contacting said spike-presenting sender cells with a plurality of receiver cells expressing angiotensin-converting enzyme 2 (ACE2) and single guide RNA (sgRNA)-Cas9 to obtain a mixture;
(d) isolating unfused receiver cells from the mixture;
(e) comparing the sgRNA abundance in unmixed receiver cells and the isolated unfused receiver cells from step (d); and
(f) identifying a factor in the receiver cell that promotes syncytium formation.

22. The method of claim 21, wherein the viral spike protein is a SARS-COV-2 (COVID-19) spike protein.

23. The method of claim 21, wherein the plurality of spike protein variants is prepared by site saturation mutagenesis (SSM).

24. The method of claim 23, wherein the SSM is over the fusion peptide proximal region (FPPR) or the furin cleavage site of SARS-COV-2.

25. The method of claim 21, wherein the syncytium-forming potential of the spike protein variants is determined by measuring a signal generated upon fusion of the sender and receiver cells.

26. The method of claim 25, wherein the signal is a fluorescent, bioluminescent, chemiluminescent or a radioactive signal.

27. The method of claim 25, wherein the signal is a green fluorescent protein (GFP) signal that is generated upon fusion of the sender and receiver cells.

28. The method of claim 21, wherein the cellular factor comprises a core regulator of clathrin-mediated endocytosis (CME).

29. The method of claim 28, wherein the CME regulator is selected from the group consisting of FCHO2, AP2M1, CAB39, RNF2 and GBP6.

30. A high throughput screening method for identifying cell fusion or syncytium formation potential of a first protein and a second protein in a biological system, the method comprising the steps of:
(a) preparing a plurality of first protein variants;
(b) incorporating said first protein variants into a plurality of first protein-presenting cells, each cell comprising a first protein variant from said plurality of first protein variants;
(c) contacting said first protein presenting cells with a plurality of cells expressing said second protein to obtain a mixture;
(d) incubating the mixture obtained in step (c) in a microfluidic system comprising multiple droplets, each droplet comprising an individual first protein presenting cell and an individual cell expressing said second protein; and
(e) determining the syncytium-forming potential of the first protein variants in the droplets.

31. The method of claim 30, wherein the biological system is a virus, tumor, maternal-fetal material exchange in the placenta, muscle contraction and bone resorption.

32. The method of claim 31 wherein the virus is selected from the group consisting of human immunodeficiency virus, Herpesviridae, respiratory syncytial virus and Coronaviridae.

33. The method of claim 30, wherein the first protein is from a tumor and the second protein is from normal somatic cells or dendritic cells.

34. The method of claim 30, wherein the first protein or second protein is from multinucleated cells selected from the group consisting of syncytiotrophoblasts, myotybes and osteoclasts.

35. The method of claim 30, wherein the first protein is from B cells and the second protein is from myeloma cells that produces hydridoma.

36. The method of claim 30, wherein the first protein is from human embryonic stem cells and the second protein is from somatic cells.

37. The method of claim 30, wherein the syncytium-forming potential of the first protein variants is determined by measuring a signal generated upon fusion of the first protein presenting cells and cells expressing said second protein.

38. The method of claim 37, wherein the signal is a fluorescent, bioluminescent, chemiluminescent or a radioactive signal.

39. The method of claim 38, wherein the signal is a green fluorescent protein (GFP) signal that is generated upon fusion of the first protein presenting cells and cells expressing said second protein.

40. The method of claim 30, wherein the syncytium-forming potential of the first protein variants is determined using deep mutational scanning (DMS) in the first protein-presenting cells.

41. The method of claim 30, further comprising the step of enriching the first protein variants having enhanced syncytium-formation potential.

42. The method of claim 30, further comprising the step of analyzing the abundance of each variant by sequencing first protein variants having enhanced syncytium-formation potential.

43. The method of claim 30, wherein the mixture in step (c) further comprises single guide RNA (sgRNA)-Cas9, and further comprising the steps of (f) isolating unfused cells expressing said second protein from the mixture; (g) comparing the sgRNA abundance in unmixed cells expressing said second protein and the isolated unfused cells expressing said second protein from step (d).

44. A high throughput screening method for identifying cell fusion or syncytium formation potential of a first protein and a second protein in a biological system, the method comprising the step of
(a) preparing a plurality of first protein variants;
(b) incorporating said first protein variants into a plurality of first protein-presenting cells, each cell comprising a first protein variant from said plurality of first protein variants;
(c) contacting said first-presenting cells with a plurality of cells expressing a second protein to obtain a mixture;
(d) performing size-exclusion selection by passing the mixture through a cell strainer; and
(e) determining the syncytium-forming potential of the first protein variants.

45. The method of claim 44, further comprising the step of collecting a first syncytia population that is retained on said strainer, and a second syncytia population that passes through the strainer.

46. The method of claim 45, further comprising the step of enriching the first protein variants having enhanced syncytium-formation potential from the first and second syncytia populations.

47. The method of claim 45, further comprising the step of analyzing the abundance of each variant by sequencing the first protein variants having enhanced syncytium-formation potential in the first and second syncytia populations.

48. The method of claim 44, wherein the plurality of the first protein variants is prepared by site saturation mutagenesis (SSM).

49. The method of claim 44, wherein the syncytium-forming potential of the first protein variants is determined by measuring a signal generated upon fusion of the first protein presenting cells and cells expressing said second protein.

50. The method of claim 49, wherein the signal is a fluorescent, bioluminescent, chemiluminescent or a radioactive signal.

51. The method of claim 50, wherein the signal is a green fluorescent protein (GFP) signal that is generated upon fusion of the first protein presenting cells and cells expressing said second protein.

52. The method of claim 44, wherein the mixture in step (c) further comprises single guide RNA (sgRNA)-Cas9, and further comprising the steps of (f) isolating unfused cells expressing said second protein from the mixture; (g) comparing the sgRNA abundance in unmixed cells expressing said second protein and the isolated unfused cells expressing said second protein.

53. The method of claim 44, wherein the syncytium-forming potential of the first protein variants is determined using deep mutational scanning (DMS) in the first protein-presenting cells.


SEQUENCE LISTING (156 sequences)

SEQ ID NO: 1 - protein, 4 aa, Severe acute respiratory syndrome-related coronavirus
PRRA

SEQ ID NO: 2 - DNA, 53 bp, synthetic construct
  source 1..53: mol_type = other DNA
acactctttccctacacgacgctcttccgatctcttgtggaaaggacgaaaca

SEQ ID NO: 3 - DNA, 54 bp, synthetic construct
  source 1..54: mol_type = other DNA
gtgactggagttcagacgtgtgctcttccgatctctaaagcgcatgctccagac

SEQ ID NO: 4 - DNA, 38 bp, synthetic construct
  source 1..38: mol_type = other DNA
cacgacgctcttccgatctcggaccccagtaaaccctc

SEQ ID NO: 5 - DNA, 44 bp, synthetic construct
  source 1..44: mol_type = other DNA
cacgacgctcttccgatctcatgtgaacaattcatacgaatgtg

SEQ ID NO: 6 - DNA, 43 bp, synthetic construct
  source 1..43: mol_type = other DNA
cagacgtgtgctcttccgatctccgaatgtccatccagacgtt

SEQ ID NO: 7 - DNA, 44 bp, synthetic construct
  source 1..44: mol_type = other DNA
cagacgtgtgctcttccgatctatttgttgggatggcaatggag

SEQ ID NO: 8 - DNA, 20 bp, synthetic construct
  source 1..20: mol_type = other DNA
aatgatacggcgaccaccga

SEQ ID NO: 9 - DNA, 21 bp, synthetic construct
  source 1..21: mol_type = other DNA
caagcagaagacggcatacga

SEQ ID NO: 10 - DNA, 69 bp, synthetic construct
  source 1..69: mol_type = other DNA
aatgatacggcgaccaccgagatctacaccgattgcaacactctttccctacacgacgctcttccgatc

SEQ ID NO: 11 - DNA, 69 bp, synthetic construct
  source 1..69: mol_type = other DNA
aatgatacggcgaccaccgagatctacacgcagaacaacactctttccctacacgacgctcttccgatc

SEQ ID NO: 12 - DNA, 69 bp, synthetic construct
  source 1..69: mol_type = other DNA
aatgatacggcgaccaccgagatctacacgccttcttacactctttccctacacgacgctcttccgatc

SEQ ID NO: 13 - DNA, 69 bp, synthetic construct
  source 1..69: mol_type = other DNA
aatgatacggcgaccaccgagatctacaccggaagaaacactctttccctacacgacgctcttccgatc

SEQ ID NO: 14 - DNA, 69 bp, synthetic construct
  source 1..69: mol_type = other DNA
aatgatacggcgaccaccgagatctacacccagagaaacactctttccctacacgacgctcttccgatc

SEQ ID NO: 15 - DNA, 69 bp, synthetic construct
  source 1..69: mol_type = other DNA
aatgatacggcgaccaccgagatctacacgaggagaaacactctttccctacacgacgctcttccgatc

SEQ ID NO: 16 - DNA, 69 bp, synthetic construct
  source 1..69: mol_type = other DNA
aatgatacggcgaccaccgagatctacaccaccagaaacactctttccctacacgacgctcttccgatc

SEQ ID NO: 17 - DNA, 69 bp, synthetic construct
  source 1..69: mol_type = other DNA
aatgatacggcgaccaccgagatctacacacgaggaaacactctttccctacacgacgctcttccgatc

SEQ ID NO: 18 - DNA, 69 bp, synthetic construct
  source 1..69: mol_type = other DNA
aatgatacggcgaccaccgagatctacacggtaggaaacactctttccctacacgacgctcttccgatc

SEQ ID NO: 19 - DNA, 69 bp, synthetic construct
  source 1..69: mol_type = other DNA
aatgatacggcgaccaccgagatctacacgaacggaaacactctttccctacacgacgctcttccgatc

SEQ ID NO: 20 - DNA, 69 bp, synthetic construct
  source 1..69: mol_type = other DNA
aatgatacggcgaccaccgagatctacacttgcggaaacactctttccctacacgacgctcttccgatc

SEQ ID NO: 21 - DNA, 69 bp, synthetic construct
  source 1..69: mol_type = other DNA
aatgatacggcgaccaccgagatctacaccagtggaaacactctttccctacacgacgctcttccgatc

SEQ ID NO: 22 - DNA, 69 bp, synthetic construct
  source 1..69: mol_type = other DNA
aatgatacggcgaccaccgagatctacactcctggaaacactctttccctacacgacgctcttccgatc

SEQ ID NO: 23 - DNA, 69 bp, synthetic construct
  source 1..69: mol_type = other DNA
aatgatacggcgaccaccgagatctacactacgcgaaacactctttccctacacgacgctcttccgatc

SEQ ID NO: 24 - DNA, 69 bp, synthetic construct
  source 1..69: mol_type = other DNA
aatgatacggcgaccaccgagatctacacagaccgaaacactctttccctacacgacgctcttccgatc

SEQ ID NO: 25 - DNA, 69 bp, synthetic construct
  source 1..69: mol_type = other DNA
aatgatacggcgaccaccgagatctacactggtcgaaacactctttccctacacgacgctcttccgatc

SEQ ID NO: 26 - DNA, 65 bp, synthetic construct
  source 1..65: mol_type = other DNA
caagcagaagacggcatacgagatttggtggagtgactggagttcagacgtgtgctcttccgatc

SEQ ID NO: 27 - DNA, 65 bp, synthetic construct
  source 1..65: mol_type = other DNA
caagcagaagacggcatacgagatttggcactgtgactggagttcagacgtgtgctcttccgatc

SEQ ID NO: 28 - DNA, 65 bp, synthetic construct
  source 1..65: mol_type = other DNA
caagcagaagacggcatacgagatttggatccgtgactggagttcagacgtgtgctcttccgatc

SEQ ID NO: 29 - DNA, 65 bp, synthetic construct
  source 1..65: mol_type = other DNA
caagcagaagacggcatacgagatttgacgtcgtgactggagttcagacgtgtgctcttccgatc

SEQ ID NO: 30 - DNA, 65 bp, synthetic construct
  source 1..65: mol_type = other DNA
caagcagaagacggcatacgagatttgagctggtgactggagttcagacgtgtgctcttccgatc

SEQ ID NO: 31 - DNA, 65 bp, synthetic construct
  source 1..65: mol_type = other DNA
caagcagaagacggcatacgagatttgaggcagtgactggagttcagacgtgtgctcttccgatc

SEQ ID NO: 32 - DNA, 65 bp, synthetic construct
  source 1..65: mol_type = other DNA
caagcagaagacggcatacgagatttatcggcgtgactggagttcagacgtgtgctcttccgatc

SEQ ID NO: 33 - DNA, 65 bp, synthetic construct
  source 1..65: mol_type = other DNA
caagcagaagacggcatacgagatttacggaggtgactggagttcagacgtgtgctcttccgatc

SEQ ID NO: 34 - DNA, 65 bp, synthetic construct
  source 1..65: mol_type = other DNA
caagcagaagacggcatacgagatttacagccgtgactggagttcagacgtgtgctcttccgatc

SEQ ID NO: 35 - DNA, 65 bp, synthetic construct
  source 1..65: mol_type = other DNA
caagcagaagacggcatacgagatttagccgagtgactggagttcagacgtgtgctcttccgatc

SEQ ID NO: 36 - DNA, 65 bp, synthetic construct
  source 1..65: mol_type = other DNA
caagcagaagacggcatacgagattctccaaggtgactggagttcagacgtgtgctcttccgatc

SEQ ID NO: 37 - DNA, 65 bp, synthetic construct
  source 1..65: mol_type = other DNA
caagcagaagacggcatacgagattctcggatgtgactggagttcagacgtgtgctcttccgatc

SEQ ID NO: 38 - DNA, 65 bp, synthetic construct
  source 1..65: mol_type = other DNA
caagcagaagacggcatacgagattctgtgcagtgactggagttcagacgtgtgctcttccgatc

SEQ ID NO: 39 - DNA, 65 bp, synthetic construct
  source 1..65: mol_type = other DNA
caagcagaagacggcatacgagattctgccatgtgactggagttcagacgtgtgctcttccgatc

SEQ ID NO: 40 - DNA, 65 bp, synthetic construct
  source 1..65: mol_type = other DNA
caagcagaagacggcatacgagattctggaacgtgactggagttcagacgtgtgctcttccgatc

SEQ ID NO: 41 - DNA, 65 bp, synthetic construct
  source 1..65: mol_type = other DNA
caagcagaagacggcatacgagattctgaggtgtgactggagttcagacgtgtgctcttccgatc

SEQ ID NO: 42 - DNA, 66 bp, synthetic construct
  source 1..66: mol_type = other DNA
ttacctcgtctcttgcttcttaccagacccaaaccaactctcctaggcgagcccggtccgtagcca

SEQ ID NO: 43 - DNA, 39 bp, synthetic construct
  source 1..39: mol_type = other DNA
ttacctcgtctcttaaatttttgggcgcatatcagatcc

SEQ ID NO: 44 - DNA, 90 bp, synthetic construct
  source 1..90: mol_type = other DNA
  misc_difference 14: note = a, c, t, g, unknown or other
  misc_difference 15: note = a, c, t, g, unknown or other
ttcgtctcttgctnnstaccagacccaaaccaactctcctaggcgtgcccggtccgtagccagtcaaagcataattgcgtacaccatgag

SEQ ID NO: 45 - DNA, 90 bp, synthetic construct
  source 1..90: mol_type = other DNA
  misc_difference 17: note = a, c, t, g, unknown or other
  misc_difference 18: note = a, c, t, g, unknown or other
ttcgtctcttgcttctnnscagacccaaaccaactctcctaggcgtgcccggtccgtagccagtcaaagcataattgcgtacaccatgag

SEQ ID NO: 46 - DNA, 90 bp, synthetic construct
  source 1..90: mol_type = other DNA
  misc_difference 20: note = a, c, t, g, unknown or other
  misc_difference 21: note = a, c, t, g, unknown or other
ttcgtctcttgcttcttacnnsacccaaaccaactctcctaggcgtgcccggtccgtagccagtcaaagcataattgcgtacaccatgag

SEQ ID NO: 47 - DNA, 90 bp, synthetic construct
  source 1..90: mol_type = other DNA
  misc_difference 23: note = a, c, t, g, unknown or other
  misc_difference 24: note = a, c, t, g, unknown or other
ttcgtctcttgcttcttaccagnnscaaaccaactctcctaggcgtgcccggtccgtagccagtcaaagcataattgcgtacaccatgag

SEQ ID NO: 48 - DNA, 90 bp, synthetic construct
  source 1..90: mol_type = other DNA
  misc_difference 26: note = a, c, t, g, unknown or other
  misc_difference 27: note = a, c, t, g, unknown or other
ttcgtctcttgcttcttaccagaccnnsaccaactctcctaggcgtgcccggtccgtagccagtcaaagcataattgcgtacaccatgag

SEQ ID NO: 49 - DNA, 90 bp, synthetic construct
  source 1..90: mol_type = other DNA
  misc_difference 29: note = a, c, t, g, unknown or other
  misc_difference 30: note = a, c, t, g, unknown or other
ttcgtctcttgcttcttaccagacccaannsaactctcctaggcgtgcccggtccgtagccagtcaaagcataattgcgtacaccatgag

SEQ ID NO: 50 - DNA, 90 bp, synthetic construct
  source 1..90: mol_type = other DNA
  misc_difference 32: note = a, c, t, g, unknown or other
  misc_difference 33: note = a, c, t, g, unknown or other
ttcgtctcttgcttcttaccagacccaaaccnnstctcctaggcgtgcccggtccgtagccagtcaaagcataattgcgtacaccatgag

SEQ ID NO: 51 - DNA, 90 bp, synthetic construct
  source 1..90: mol_type = other DNA
  misc_difference 35: note = a, c, t, g, unknown or other
  misc_difference 36: note = a, c, t, g, unknown or other
ttcgtctcttgcttcttaccagacccaaaccaacnnscctaggcgtgcccggtccgtagccagtcaaagcataattgcgtacaccatgag

SEQ ID NO: 52 - DNA, 90 bp, synthetic construct
  source 1..90: mol_type = other DNA
  misc_difference 38: note = a, c, t, g, unknown or other
  misc_difference 39: note = a, c, t, g, unknown or other
ttcgtctcttgcttcttaccagacccaaaccaactctnnsaggcgtgcccggtccgtagccagtcaaagcataattgcgtacaccatgag

SEQ ID NO: 53 - DNA, 90 bp, synthetic construct
  source 1..90: mol_type = other DNA
  misc_difference 41: note = a, c, t, g, unknown or other
  misc_difference 42: note = a, c, t, g, unknown or other
ttcgtctcttgcttcttaccagacccaaaccaactctcctnnscgtgcccggtccgtagccagtcaaagcataattgcgtacaccatgag

SEQ ID NO: 54 - DNA, 90 bp, synthetic construct
  source 1..90: mol_type = other DNA
  misc_difference 44: note = a, c, t, g, unknown or other
  misc_difference 45: note = a, c, t, g, unknown or other
ttcgtctcttgcttcttaccagacccaaaccaactctcctaggnnsgcccggtccgtagccagtcaaagcataattgcgtacaccatgag

SEQ ID NO: 55 - DNA, 90 bp, synthetic construct
  source 1..90: mol_type = other DNA
  misc_difference 47: note = a, c, t, g, unknown or other
  misc_difference 48: note = a, c, t, g, unknown or other
ttcgtctcttgcttcttaccagacccaaaccaactctcctaggcgtnnscggtccgtagccagtcaaagcataattgcgtacaccatgag

SEQ ID NO: 56 - DNA, 90 bp, synthetic construct
  source 1..90: mol_type = other DNA
  misc_difference 50: note = a, c, t, g, unknown or other
  misc_difference 51: note = a, c, t, g, unknown or other
ttcgtctcttgcttcttaccagacccaaaccaactctcctaggcgtgccnnstccgtagccagtcaaagcataattgcgtacaccatgag

SEQ ID NO: 57 - DNA, 90 bp, synthetic construct
  source 1..90: mol_type = other DNA
  misc_difference 53: note = a, c, t, g, unknown or other
  misc_difference 54: note = a, c, t, g, unknown or other
ttcgtctcttgcttcttaccagacccaaaccaactctcctaggcgtgcccggnnsgtagccagtcaaagcataattgcgtacaccatgag

SEQ ID NO: 58 - DNA, 90 bp, synthetic construct
  source 1..90: mol_type = other DNA
  misc_difference 56: note = a, c, t, g, unknown or other
  misc_difference 57: note = a, c, t, g, unknown or other
ttcgtctcttgcttcttaccagacccaaaccaactctcctaggcgtgcccggtccnnsgccagtcaaagcataattgcgtacaccatgag

SEQ ID NO: 59 - DNA, 90 bp, synthetic construct
  source 1..90: mol_type = other DNA
  misc_difference 59: note = a, c, t, g, unknown or other
  misc_difference 60: note = a, c, t, g, unknown or other
ttcgtctcttgcttcttaccagacccaaaccaactctcctaggcgtgcccggtccgtannsagtcaaagcataattgcgtacaccatgag

SEQ ID NO: 60 - DNA, 90 bp, synthetic construct
  source 1..90: mol_type = other DNA
  misc_difference 62: note = a, c, t, g, unknown or other
  misc_difference 63: note = a, c, t, g, unknown or other
ttcgtctcttgcttcttaccagacccaaaccaactctcctaggcgtgcccggtccgtagccnnscaaagcataattgcgtacaccatgag

SEQ ID NO: 61 - DNA, 90 bp, synthetic construct
  source 1..90: mol_type = other DNA
  misc_difference 65: note = a, c, t, g, unknown or other
  misc_difference 66: note = a, c, t, g, unknown or other
ttcgtctcttgcttcttaccagacccaaaccaactctcctaggcgtgcccggtccgtagccagtnnsagcataattgcgtacaccatgag

SEQ ID NO: 62 - DNA, 90 bp, synthetic construct
  source 1..90: mol_type = other DNA
  misc_difference 68: note = a, c, t, g, unknown or other
  misc_difference 69: note = a, c, t, g, unknown or other
ttcgtctcttgcttcttaccagacccaaaccaactctcctaggcgtgcccggtccgtagccagtcaannsataattgcgtacaccatgag

SEQ ID NO: 63 - DNA, 90 bp, synthetic construct
  source 1..90: mol_type = other DNA
  misc_difference 15: note = a, c, t, g, unknown or other
  misc_difference 16: note = a, c, t, g, unknown or other
ttcgtctcttaaasnnttgggcgcatatcagatcccgcgccgcgatgtcgcccaaacaatcaccgtattgtttaataaagccagcatcgg

SEQ ID NO: 64 - DNA, 90 bp, synthetic construct
  source 1..90: mol_type = other DNA
  misc_difference 18: note = a, c, t, g, unknown or other
  misc_difference 19: note = a, c, t, g, unknown or other
ttcgtctcttaaatttsnnggcgcatatcagatcccgcgccgcgatgtcgcccaaacaatcaccgtattgtttaataaagccagcatcgg

SEQ ID NO: 65 - DNA, 90 bp, synthetic construct
  source 1..90: mol_type = other DNA
  misc_difference 21: note = a, c, t, g, unknown or other
  misc_difference 22: note = a, c, t, g, unknown or other
ttcgtctcttaaatttttgsnngcatatcagatcccgcgccgcgatgtcgcccaaacaatcaccgtattgtttaataaagccagcatcgg

SEQ ID NO: 66 - DNA, 90 bp, synthetic construct
  source 1..90: mol_type = other DNA
  misc_difference 24: note = a, c, t, g, unknown or other
  misc_difference 25: note = a, c, t, g, unknown or other
ttcgtctcttaaatttttgggcsnntatcagatcccgcgccgcgatgtcgcccaaacaatcaccgtattgtttaataaagccagcatcgg

SEQ ID NO: 67 - DNA, 90 bp, synthetic construct
  source 1..90: mol_type = other DNA
  misc_difference 27: note = a, c, t, g, unknown or other
  misc_difference 28: note = a, c, t, g, unknown or other
ttcgtctcttaaatttttgggcgcasnncagatcccgcgccgcgatgtcgcccaaacaatcaccgtattgtttaataaagccagcatcgg

SEQ ID NO: 68 - DNA, 90 bp, synthetic construct
  source 1..90: mol_type = other DNA
  misc_difference 30: note = a, c, t, g, unknown or other
  misc_difference 31: note = a, c, t, g, unknown or other
ttcgtctcttaaatttttgggcgcatatsnnatcccgcgccgcgatgtcgcccaaacaatcaccgtattgtttaataaagccagcatcgg

SEQ ID NO: 69 - DNA, 90 bp, synthetic construct
  source 1..90: mol_type = other DNA
  misc_difference 33: note = a, c, t, g, unknown or other
  misc_difference 34: note = a, c, t, g, unknown or other
ttcgtctcttaaatttttgggcgcatatcagsnnccgcgccgcgatgtcgcccaaacaatcaccgtattgtttaataaagccagcatcgg

SEQ ID NO: 70 - DNA, 90 bp, synthetic construct
  source 1..90: mol_type = other DNA
  misc_difference 36: note = a, c, t, g, unknown or other
  misc_difference 37: note = a, c, t, g, unknown or other
ttcgtctcttaaatttttgggcgcatatcagatcsnncgccgcgatgtcgcccaaacaatcaccgtattgtttaataaagccagcatcgg

SEQ ID NO: 71 - DNA, 90 bp, synthetic construct
  source 1..90: mol_type = other DNA
  misc_difference 39: note = a, c, t, g, unknown or other
  misc_difference 40: note = a, c, t, g, unknown or other
ttcgtctcttaaatttttgggcgcatatcagatcccgsnncgcgatgtcgcccaaacaatcaccgtattgtttaataaagccagcatcgg

SEQ ID NO: 72 - DNA, 90 bp, synthetic construct
  source 1..90: mol_type = other DNA
  misc_difference 42: note = a, c, t, g, unknown or other
  misc_difference 43: note = a, c, t, g, unknown or other
ttcgtctcttaaatttttgggcgcatatcagatcccgcgcsnngatgtcgcccaaacaatcaccgtattgtttaataaagccagcatcgg

SEQ ID NO: 73 - DNA, 90 bp, synthetic construct
  source 1..90: mol_type = other DNA
  misc_difference 45: note = a, c, t, g, unknown or other
  misc_difference 46: note = a, c, t, g, unknown or other
ttcgtctcttaaatttttgggcgcatatcagatcccgcgccgcsnngtcgcccaaacaatcaccgtattgtttaataaagccagcatcgg

SEQ ID NO: 74 - DNA, 90 bp, synthetic construct
  source 1..90: mol_type = other DNA
  misc_difference 48: note = a, c, t, g, unknown or other
  misc_difference 49: note = a, c, t, g, unknown or other
ttcgtctcttaaatttttgggcgcatatcagatcccgcgccgcgatsnngcccaaacaatcaccgtattgtttaataaagccagcatcgg

SEQ ID NO: 75 - DNA, 90 bp, synthetic construct
  source 1..90: mol_type = other DNA
  misc_difference 51: note = a, c, t, g, unknown or other
  misc_difference 52: note = a, c, t, g, unknown or other
ttcgtctcttaaatttttgggcgcatatcagatcccgcgccgcgatgtcsnncaaacaatcaccgtattgtttaataaagccagcatcgg

SEQ ID NO: 76 - DNA, 90 bp, synthetic construct
  source 1..90: mol_type = other DNA
  misc_difference 54: note = a, c, t, g, unknown or other
  misc_difference 55: note = a, c, t, g, unknown or other
ttcgtctcttaaatttttgggcgcatatcagatcccgcgccgcgatgtcgccsnnacaatcaccgtattgtttaataaagccagcatcgg

SEQ ID NO: 77 - DNA, 90 bp, synthetic construct
  source 1..90: mol_type = other DNA
  misc_difference 57: note = a, c, t, g, unknown or other
  misc_difference 58: note = a, c, t, g, unknown or other
ttcgtctcttaaatttttgggcgcatatcagatcccgcgccgcgatgtcgcccaasnnatcaccgtattgtttaataaagccagcatcgg

SEQ ID NO: 78 - DNA, 90 bp, synthetic construct
  source 1..90: mol_type = other DNA
  misc_difference 60: note = a, c, t, g, unknown or other
  misc_difference 61: note = a, c, t, g, unknown or other
ttcgtctcttaaatttttgggcgcatatcagatcccgcgccgcgatgtcgcccaaacasnnaccgtattgtttaataaagccagcatcgg

SEQ ID NO: 79 - DNA, 90 bp, synthetic construct
  source 1..90: mol_type = other DNA
  misc_difference 63: note = a, c, t, g, unknown or other
  misc_difference 64: note = a, c, t, g, unknown or other
ttcgtctcttaaatttttgggcgcatatcagatcccgcgccgcgatgtcgcccaaacaatcsnngtattgtttaataaagccagcatcgg

SEQ ID NO: 80 - DNA, 90 bp, synthetic construct
  source 1..90: mol_type = other DNA
  misc_difference 66: note = a, c, t, g, unknown or other
  misc_difference 67: note = a, c, t, g, unknown or other
ttcgtctcttaaatttttgggcgcatatcagatcccgcgccgcgatgtcgcccaaacaatcaccsnnttgtttaataaagccagcatcgg

SEQ ID NO: 81 - DNA, 90 bp, synthetic construct
  source 1..90: mol_type = other DNA
  misc_difference 69: note = a, c, t, g, unknown or other
  misc_difference 70: note = a, c, t, g, unknown or other
ttcgtctcttaaatttttgggcgcatatcagatcccgcgccgcgatgtcgcccaaacaatcaccgtasnntttaataaagccagcatcgg

SEQ ID NO: 82 - DNA, 90 bp, synthetic construct
  source 1..90: mol_type = other DNA
ttcgtctcttaaagtgttgggcgcatatcagatcccgcgccgcgatgtcgcccaaacaatcaccgtattgtttaataaagccagcatcgg

SEQ ID NO: 83 - DNA, 90 bp, synthetic construct
  source 1..90: mol_type = other DNA
ttcgtctcttaaatttttgggcgcatatcagatcccgccacgcgatgtcgcccaaacaatcaccgtattgtttaataaagccagcatcgg

SEQ ID NO: 84 - DNA, 90 bp, synthetic construct
  source 1..90: mol_type = other DNA
ttcgtctcttaaagcattgggcgcatatcagatcccgcgccgcgatgtcgcccaaacaatcaccgtattgtttaataaagccagcatcgg

SEQ ID NO: 85 - DNA, 90 bp, synthetic construct
  source 1..90: mol_type = other DNA
ttcgtctcttaaatttttgcatgcatatcagatcccgcgccgcgatgtcgcccaaacaatcaccgtattgtttaataaagccagcatcgg

SEQ ID NO: 86 - DNA, 90 bp, synthetic construct
  source 1..90: mol_type = other DNA
ttcgtctcttaaacagttgggcgcatatcagatcccgcgccgcgatgtcgcccaaacaatcaccgtattgtttaataaagccagcatcgg

SEQ ID NO: 87 - DNA, 90 bp, synthetic construct
  source 1..90: mol_type = other DNA
ttcgtctcttaaatttcacggcgcatatcagatcccgcgccgcgatgtcgcccaaacaatcaccgtattgtttaataaagccagcatcgg

SEQ ID NO: 88 - DNA, 90 bp, synthetic construct
  source 1..90: mol_type = other DNA
ttcgtctcttaaagttttgggcgcatatcagatcccgcgccgcgatgtcgcccaaacaatcaccgtattgtttaataaagccagcatcgg

SEQ ID NO: 89 - DNA, 90 bp, synthetic construct
  source 1..90: mol_type = other DNA
ttcgtctcttaaatttttgggcgcatatcagatcccgcgccgcgatcatgcccaaacaatcaccgtattgtttaataaagccagcatcgg

SEQ ID NO: 90 - DNA, 90 bp, synthetic construct
  source 1..90: mol_type = other DNA
ttcgtctcttaaatttttgggcgcatatcagatcccgcgccgcgatgtcgcccaaacaatcgtagtattgtttaataaagccagcatcgg

SEQ ID NO: 91 - DNA, 90 bp, synthetic construct
  source 1..90: mol_type = other DNA
ttcgtctcttaaatttttgggcgcatatcagatcccgcgccgcgatgtcgcccaaacaatcaccgaattgtttaataaagccagcatcgg

SEQ ID NO: 92 - DNA, 90 bp, synthetic construct
  source 1..90: mol_type = other DNA
ttcgtctcttaaaccattgggcgcatatcagatcccgcgccgcgatgtcgcccaaacaatcaccgtattgtttaataaagccagcatcgg

SEQ ID NO: 93 - DNA, 90 bp, synthetic construct
  source 1..90: mol_type = other DNA
ttcgtctcttaaatttttgggcgcatatcagatcccgcgccgcgatgtcgcccaagctatcaccgtattgtttaataaagccagcatcgg

SEQ ID NO: 94 - DNA, 90 bp, synthetic construct
  source 1..90: mol_type = other DNA
ttcgtctcttaaatttttgggcgcatatcaggttccgcgccgcgatgtcgcccaaacaatcaccgtattgtttaataaagccagcatcgg

SEQ ID NO: 95 - DNA, 90 bp, synthetic construct
  source 1..90: mol_type = other DNA
ttcgtctcttaaatttttgggcgcttatcagatcccgcgccgcgatgtcgcccaaacaatcaccgtattgtttaataaagccagcatcgg

SEQ ID NO: 96 - DNA, 90 bp, synthetic construct
  source 1..90: mol_type = other DNA
ttcgtctcttgcttcttaccagacccaaaccaactcttacaggcgtgcccggtccgtagccagtcaaagcataattgcgtacaccatgag

SEQ ID NO: 97 - DNA, 90 bp, synthetic construct
  source 1..90: mol_type = other DNA
ttcgtctcttgcttcttaccagacccaaaccaactctcctaggcacgcccggtccgtagccagtcaaagcataattgcgtacaccatgag

SEQ ID NO: 98 - DNA, 90 bp, synthetic construct
  source 1..90: mol_type = other DNA
ttcgtctcttgcttcttaccagacccaaaccaactctcctaggcgacaccggtccgtagccagtcaaagcataattgcgtacaccatgag

SEQ ID NO: 99 - DNA, 90 bp, synthetic construct
  source 1..90: mol_type = other DNA
ttcgtctcttgcttcttaccagacccaaaccaactctcctaggcgtgccttctccgtagccagtcaaagcataattgcgtacaccatgag

SEQ ID NO: 100 - DNA, 90 bp, synthetic construct
  source 1..90: mol_type = other DNA
ttcgtctcttgcttcttaccagacccaaaccaactctcctaggcgtgcccggtccatcgccagtcaaagcataattgcgtacaccatgag

SEQ ID NO: 101 - DNA, 90 bp, synthetic construct
  source 1..90: mol_type = other DNA
ttcgtctcttgcttcttaccagacccaaaccaactctcctaggcgtgcccggtccgtaaccagtcaaagcataattgcgtacaccatgag

SEQ ID NO: 102 - DNA, 36 bp, synthetic construct
  source 1..36: mol_type = other DNA
ttcgtctcttgaagtgttgagcacagataaggtcgc

SEQ ID NO: 103 - DNA, 61 bp, synthetic construct
  source 1..61: mol_type = other DNA
ttcgtctcttgaatttttgagcacagataaggtcgcgccacgcaatgtctcccaaacaatc

SEQ ID NO: 104 - DNA, 81 bp, synthetic construct
  source 1..81: mol_type = other DNA
ttcgtctcttgaatttttgagcacagataaggtcgcgggccgcaatgtctcccaagctatccccatactgcttaatgaaac

SEQ ID NO: 105 - DNA, 50 bp, synthetic construct
  source 1..50: mol_type = other DNA
ttcgtctcttgaatttttgagcacagataaggttgcgggccgcaatgtct

SEQ ID NO: 106 - DNA, 42 bp, synthetic construct
  source 1..42: mol_type = other DNA
ttcgtctcttgaatttttgagcgctgataaggtcgcgggccg

SEQ ID NO: 107 - DNA, 22 bp, synthetic construct
  source 1..22: mol_type = other DNA
gtctttctcgctacctggtacg

SEQ ID NO: 108 - DNA, 22 bp, synthetic construct
  source 1..22: mol_type = other DNA
ggtcctgagtctcagacaaagc

SEQ ID NO: 109 - DNA, 20 bp, synthetic construct
  source 1..20: mol_type = other DNA
cgcatacagtcttrcaggct

SEQ ID NO: 110 - DNA, 24 bp, synthetic construct
  source 1..24: mol_type = other DNA
gtgtgatgttgawatgacatggtc

SEQ ID NO: 111 - DNA, 28 bp, synthetic construct
  source 1..28: mol_type = other DNA
  modified_base 1: mod_base = OTHER; note = Fluorescein modified nucleotide
  modified_base 28: mod_base = OTHER; note = Iowa Black FQ modified nucleotide
ttaagatgtggtgcttgcatacgtagac

SEQ ID NO: 112 - DNA, 20 bp, synthetic construct
  source 1..20: mol_type = other DNA
attccacccatggcaaattc

SEQ ID NO: 113 - DNA, 20 bp, synthetic construct
  source 1..20: mol_type = other DNA
cgctcctggaagatggtgat

SEQ ID NO: 114 - DNA, 21 bp, synthetic construct
  source 1..21: mol_type = other DNA
acggccaggtcatcactattg

SEQ ID NO: 115 - DNA, 21 bp, synthetic construct
  source 1..21: mol_type = other DNA
caagaaggaaggctggaaaag

SEQ ID NO: 116 - DNA, 20 bp, synthetic construct
  source 1..20: mol_type = other DNA
acagtttagactacaatgag

SEQ ID NO: 117 - DNA, 19 bp, synthetic construct
  source 1..19: mol_type = other DNA
atcttcaatcaactggccg

SEQ ID NO: 118 - DNA, 19 bp, synthetic construct
  source 1..19: mol_type = other DNA
taatagggcaaaaaagacg

SEQ ID NO: 119 - DNA, 19 bp, synthetic construct
  source 1..19: mol_type = other DNA
agctgtaaatcagctacca

SEQ ID NO: 120 - DNA, 19 bp, synthetic construct
  source 1..19: mol_type = other DNA
acccacagaattccgagac

SEQ ID NO: 121 - DNA, 19 bp, synthetic construct
  source 1..19: mol_type = other DNA
tactttggcaagatcagcg

SEQ ID NO: 122 - DNA, 19 bp, synthetic construct
  source 1..19: mol_type = other DNA
tttaacagagctaccatag

SEQ ID NO: 123 - DNA, 18 bp, synthetic construct
  source 1..18: mol_type = other DNA
ctagttttgtcattgacc

SEQ ID NO: 124 - DNA, 19 bp, synthetic construct
  source 1..19: mol_type = other DNA
cttctagccaatattgaga

SEQ ID NO: 125 - DNA, 19 bp, synthetic construct
  source 1..19: mol_type = other DNA
aaattggatgtttgaactc

SEQ ID NO: 126 - DNA, 19 bp, synthetic construct
  source 1..19: mol_type = other DNA
gagttacaacgaacacctc

SEQ ID NO: 127 - DNA, 19 bp, synthetic construct
  source 1..19: mol_type = other DNA
attcactgtgtagacttcg

SEQ ID NO: 128 - DNA, 19 bp, synthetic construct
  source 1..19: mol_type = other DNA
tgaatgggcgaccaagaac

SEQ ID NO: 129 - DNA, 19 bp, synthetic construct
  source 1..19: mol_type = other DNA
tacttaaagtggcggtaga

SEQ ID NO: 130 - DNA, 19 bp, synthetic construct
  source 1..19: mol_type = other DNA
ccgtgggatctgtaggcaa

SEQ ID NO: 131 - DNA, 19 bp, synthetic construct
  source 1..19: mol_type = other DNA
cactcacaccggtggccct

SEQ ID NO: 132 - DNA, 19 bp, synthetic construct
  source 1..19: mol_type = other DNA
agtgatgtctggcacgcgg

SEQ ID NO: 133 - DNA, 19 bp, synthetic construct
  source 1..19: mol_type = other DNA
gacccgtgaccctcgtgag

SEQ ID NO: 134 - DNA, 20 bp, synthetic construct
  source 1..20: mol_type = other DNA
ccattcagtctgaaatacgg

SEQ ID NO: 135 - DNA, 19 bp, synthetic construct
  source 1..19: mol_type = other DNA
cctcatccacgaaagatgc

SEQ ID NO: 136 - DNA, 19 bp, synthetic construct
  source 1..19: mol_type = other DNA
tacgctgcgacacccggtg

SEQ ID NO: 137 - DNA, 20 bp, synthetic construct
  source 1..20: mol_type = other DNA
agagtttagactacaatgag

SEQ ID NO: 138 - DNA, 20 bp, synthetic construct
  source 1..20: mol_type = other DNA
caagagatggtccaaatagg

SEQ ID NO: 139 - DNA, 21 bp, synthetic construct
  source 1..21: mol_type = other DNA
gcagaagaatcaacgttattt

SEQ ID NO: 140 - DNA, 21 bp, synthetic construct
  source 1..21: mol_type = other DNA
ctggagggaagttacatatta

SEQ ID NO: 141 - DNA, 21 bp, synthetic construct
  source 1..21: mol_type = other DNA
ggagggaagttacatattatt

SEQ ID NO: 142 - DNA, 21 bp, synthetic construct
  source 1..21: mol_type = other DNA
cgagggcgacttaaccttagg

SEQ ID NO: 143 - protein, 40 aa, Severe acute respiratory syndrome-related coronavirus
VTLADAGFMKQYGECLGDINARDLICAQKFNGLTVLPPLL

SEQ ID NO: 144 - protein, 42 aa, Middle East respiratory syndrome-related coronavirus
VTIADPGYMQGYDDCMQQGPASARDLICAQYVAGYKVLPPLM

SEQ ID NO: 145 - protein, 45 aa, Rousettus bat coronavirus GCCDC1
VVTSGLGTVDQDYKNCVGKLGVAQDIADVGCAQYYNGIMVLPGVV

SEQ ID NO: 146 - protein, 41 aa, Bat coronavirus CDPHE15
VVTSGLGTVDADYKACSNGLSIADLVCAQYYQGVMVLPGVV

SEQ ID NO: 147 - protein, 43 aa, Bat coronavirus
IVTSGLGTVDDDYKECAAKMANTIAEAGCVQYYNGIMVLPGVV

SEQ ID NO: 148 - protein, 40 aa, Severe acute respiratory syndrome coronavirus 2
VTLADAGFIKQYGDCLGDIAARDLICAQKFNGLTVLPPLL

SEQ ID NO: 149 - protein, 40 aa, Bat coronavirus; identical to SEQ ID NO: 148

SEQ ID NO: 150 - protein, 37 aa, Severe acute respiratory syndrome-related coronavirus
YECDIPIGAGICASYHTVSLLRSTSQKSIVAYTMSLG

SEQ ID NO: 151 - protein, 39 aa, Middle East respiratory syndrome-related coronavirus
EDCKLPLGQSLCALPDTPSTLTPRSVRSVPGEMRLASIA

SEQ ID NO: 152 - protein, 34 aa, Rousettus bat coronavirus GCCDC1
GGCQTPVLEYGSMGLCADGSVVDVTLRQYQPAPV

SEQ ID NO: 153 - protein, 33 aa, Bat coronavirus CDPHE15
SSCSEPVLTYSSLGVCKDGALRILQPREDTTQP

SEQ ID NO: 154 - protein, 33 aa, Bat coronavirus
ELCKNPSLMYGGLGVCEDGRLVNISRSEDTFVP

SEQ ID NO: 155 - protein, 41 aa, Severe acute respiratory syndrome coronavirus 2
YECDIPIGAGICASYQTQTNSPRRARSVASQSIIAYTMSLG

SEQ ID NO: 156 - protein, 37 aa, Bat coronavirus
YECDIPIGAGICASYQTQTNSRSVASQSIIAYTMSLG